SEQUENCE LISTING (38 sequences)

SEQ ID NO: 1 - DNA, 105 bp, Homo sapiens
cgctctatccgtgcccagcgccacactgacatgcccaagactcagaaggaagtacatttgaagaacacaagtagaggaagtgcaggaaacaagacctacagaatg

SEQ ID NO: 2 - protein, 35 aa, Homo sapiens
RSIRAQRHTDMPKTQKEVHLKNTSRGSAGNKTYRM

SEQ ID NO: 3 - DNA, 123 bp, Homo sapiens
cgctctatccgtgcccagcgccacactgacatgcccaagactcagaagtccccgtccctatcgacaaacaagaaaacgaagctgcaaaggagaaggaaaggaagtacatttgaagaacacaag

SEQ ID NO: 4 - protein, 41 aa, Homo sapiens
RSIRAQRHTDMPKTQKSPSLSTNKKTKLQRRRKGSTFEEHK

SEQ ID NO: 5 - DNA, 138 bp, Homo sapiens
atggggaaaatcagcagccttccaactcaattatttaagatctgcctctgtgacttcttgaagataaagatacacatcatgtcgtcttcacacctcttctacctggcgctctgcttgctcaccttcaccagctccacc

SEQ ID NO: 6 - protein, 46 aa, Homo sapiens
MGKISSLPTQLFKICLCDFLKIKIHIMSSSHLFYLALCLLTFTSST

SEQ ID NO: 7 - DNA, 90 bp, Homo sapiens
atgaccgcacctgcaataaagatacacatcatgtcgtcttcacacctcttctacctggcgctctgcttgctcaccttcaccagctccacc

SEQ ID NO: 8 - protein, 30 aa, Homo sapiens
MTAPAIKIHIMSSSHLFYLALCLLTFTSST

SEQ ID NO: 9 - DNA, 60 bp, Homo sapiens
atgtcgtcttcacacctcttctacctggcgctctgcttgctcaccttcaccagctccacc

SEQ ID NO: 10 - protein, 20 aa, Homo sapiens
MSSSHLFYLALCLLTFTSST

SEQ ID NO: 11 - DNA, 216 bp, Homo sapiens
acagctggaccagagaccctttgcggggctgagctggtggatgctcttcagttcgtgtgtggaccgaggggcttttacttcaacaagcccacaggctatggctccagcattcggagggcacctcagacaggcattgtggatgagtgttgcttccggagctgtgatctgaggagactggagatgtactgtgccccactgaagcctacaaaagcagcc

SEQ ID NO: 12 - protein, 72 aa, Homo sapiens
TAGPETLCGAELVDALQFVCGPRGFYFNKPTGYGSSIRRAPQTGIVDECCFRSCDLRRLEMYCAPLKPTKAA

SEQ ID NO: 13 - DNA, 25 bp, Artificial Sequence
  oligonucleotide
acatcaacaagagcttgacccaggc

SEQ ID NO: 14 - DNA, 24 bp, Artificial Sequence
  oligonucleotide
agctcatatggtccgacagcacga

SEQ ID NO: 15 - DNA, 24 bp, Artificial Sequence
  oligonucleotide
aggataccactcccaacagacgtg

SEQ ID NO: 16 - DNA, 24 bp, Artificial Sequence
  oligonucleotide
gtagctatggtactccagaagacc

SEQ ID NO: 17 - DNA, 19 bp, Artificial Sequence
  oligonucleotide
ccaagccttatcggaaatg

SEQ ID NO: 18 - DNA, 16 bp, Artificial Sequence
  oligonucleotide
tggccttgtagacacc

SEQ ID NO: 19 - DNA, 15 bp, Artificial Sequence
  oligonucleotide
catcggcattttgaa

SEQ ID NO: 20 - DNA, 20 bp, Artificial Sequence
  oligonucleotide
cgtttggcacatccatctcc

SEQ ID NO: 21 - DNA, 17 bp, Artificial Sequence
  oligonucleotide
tgggtgtgaaccacgaa

SEQ ID NO: 22 - DNA, 16 bp, Artificial Sequence
  oligonucleotide
acagctttccagaggg

SEQ ID NO: 23 - DNA, 24 bp, Artificial Sequence
  oligonucleotide
atgggctccttctccatcaccctg

SEQ ID NO: 24 - DNA, 23 bp, Artificial Sequence
  oligonucleotide
tcggtaccggaagctgttgcagc

SEQ ID NO: 25 - DNA, 24 bp, Artificial Sequence
  oligonucleotide
atggatctcctgaaggtgctgtcc

SEQ ID NO: 26 - DNA, 24 bp, Artificial Sequence
  oligonucleotide
gcgttacagcccaaacgactgacg

SEQ ID NO: 27 - DNA, 27 bp, Artificial Sequence
  oligonucleotide
ctgagcagaagcgcaatgcagagtcgg

SEQ ID NO: 28 - DNA, 28 bp, Artificial Sequence
  oligonucleotide
ctcctcattcaggcccttggcaccaatg

SEQ ID NO: 29 - DNA, 24 bp, Artificial Sequence
  oligonucleotide
atgtgcgacgaagacgagaccacc

SEQ ID NO: 30 - DNA, 24 bp, Artificial Sequence
  oligonucleotide
gccacatacatggcaggcacgttg

SEQ ID NO: 31 - DNA, 24 bp, Artificial Sequence
  oligonucleotide
gatcccagcagcaagaaggtgacg

SEQ ID NO: 32 - DNA, 24 bp, Artificial Sequence
  oligonucleotide
tggagaagcccataagcacagcag

SEQ ID NO: 33 - DNA, 25 bp, Artificial Sequence
  oligonucleotide
taaaacgcagctcagtaacagtccg

SEQ ID NO: 34 - DNA, 25 bp, Artificial Sequence
  oligonucleotide
tggaatcctgtggcatccatgaaac

SEQ ID NO: 35 - DNA, 23 bp, Artificial Sequence
  oligonucleotide
cccagaccctcacacactcagat

SEQ ID NO: 36 - DNA, 21 bp, Artificial Sequence
  oligonucleotide
ttgtcccttgaagagaacctg

SEQ ID NO: 37 - DNA, 20 bp, Artificial Sequence
  oligonucleotide
gtgggccgctctaggcacaa

SEQ ID NO: 38 - DNA, 24 bp, Artificial Sequence
  oligonucleotide
ctctttgatgtcacgcacgatttc

(19) United States
(12) Patent Application Publication
(10) Pub. No.: US 2009/0038022 A1
(43) Pub. Date: Feb. 5, 2009

(54) IGF-1 Novel peptides

(76) Inventors: Nadia Rosenthal, Heidelberg (DE); Antonio Musaro, Rome (IT)

Correspondence Address: WENDEROTH, LIND & PONACK, L.L.P., 2033 K STREET N. W., SUITE 800, WASHINGTON, DC, 20006-1021, US

(21) Appl. No.: 11/791,722

(22) PCT Filed: Nov. 29, 2005
(86) PCT No.: PCT/IB05/03953

§ 371 Date: Jul. 24, 2007

(30) Foreign Application Priority Data
Nov. 29, 2004 (GB) 0426154.1

(51) Int. Cl. A01K 67/027; C07K 2/00; C07H 21/04; C12N 15/63; C12N 5/10; A01N 1/02; A61K 38/02; A61K 31/711; A61K 35/12; A61K 38/48

(52) U.S. Cl. 800/13; 530/300; 536/23.5; 435/320.1; 435/325; 435/375; 435/377; 435/1.1; 514/2; 514/44; 424/93.7; 424/94.64

(57) ABSTRACT

The present invention relates to novel polypeptide constructs based on peptides derived from Insulin-like Growth Factor I (IGF-1). The invention also relates to novel uses for IGF-1-derived peptides, particularly for the prevention and treatment of diseases involving regulation of cellular growth or differentiation, regeneration and tissue repair.

The present invention relates to novel polypeptide constructs based on peptides derived from Insulin-like Growth Factor I (IGF-I). The invention also relates to novel uses for IGF-1-derived peptides, particularly for the prevention and treatment of diseases involving regulation of cellular growth or differentiation, regeneration and tissue repair.

All documents mentioned in the text and listed at the end of the description are incorporated herein by reference.

Insulin-like growth factors (IGFs) are members of the highly diverse insulin gene family that includes insulin, IGF-I, IGF-II, relaxin, prothoraciotropic hormone (PTTH), and molluscan insulin-related peptide (1;2;3). The IGFs are circulating, mitogenic peptide hormones that have an important role in stimulating growth, differentiation, metabolism and regeneration both in vitro and in vivo (4;5).

The Insulin-like growth factor-1 (IGF-1) gene gives rise to several isoforms of unprocessed (precursor) IGF-1 which differ by the length of the amino terminal leader (signal) peptide and structure of the carboxy terminal end (E-domain) (discussed in detail below). These unprocessed polypeptides undergo post-translational protease cleavage to remove the leader sequence and the E-domain to yield a 70 amino acid long (mol wt 7,649 D) single chain mature IGF-1 polypeptide with three intrachain disulphide bridges.

The IGF-1 gene gives rise to a heterogeneous pool of mRNA transcripts (FIG. 1B). Such heterogeneity of the mRNAs results from several events (or combination of these events): use of alternative transcription start sites located in leader exons (exon 1 and exon 2) (6; 7; 8); alternative post-transcriptional exon splicing (9; 10; 11; 6; 7; 8); and use of different polyadenylation sites (12; 13). These multiple IGF-1 mRNAs transcripts encode different isoforms of precursor IGF-1 peptide (FIG. 1C), which undergo post-translational cleavage to release the biologically active mature (70 amino acid long) IGF-1. There is also a degree of heterogeneity in the signal peptides used, that are eventually cleaved during post-translational processing (8). Additionally, alternative splicing of exons at the 3′-end of mRNA precursor introduces further complexity in the variety of IGF-1 transcripts and IGF-1 isoforms translated from these transcripts.

Adding to the confusion in the literature regarding the large array of IGF-I isoforms, IGF-1 functions have also been extensively analysed, with several groups reporting different and contradictory roles of the growth factor in vivo and in vitro. For example, Ito and co-workers showed that IGF-1, lacking class 1 or 2 signal peptides or C-terminal E peptides, increases the transcription of muscle specific genes, and induces a two fold increase of cell size in neonatal rat cardiomyocytes (14) indicating a functional role in regulating cardiac cells hypertrophy. Conversely to this study, cultured neonatal cardiomyocytes treated with an antisense probe to IGF-1 receptor, showed suppressed DNA replication, mitosis and cell proliferation. Moreover, the antisense treatment did not alter the expression of ANF in myocytes or cellular hypertrophy (15).

In vivo, intravenous infusion of IGF-1, lacking class 1 or 2 signal peptides or E peptides, has been shown to induce a significant increase in protein synthesis particularly in the heart, which was not accompanied by significant changes in blood glucose (16). Interestingly, administration of IGF-1 alone or in combination with Growth Hormone (GH) in normal adult rats increased the left ventricular weight compared with placebo-treated rats (17). Transgenic mice generated with the human IGF-1B cDNA showed no striking differences in heart size and cell volume when compared to control mice, but the number of myocytes in the heart was 55% higher in transgenic animals, indicating that IGF-1 overexpression is coupled with myocyte proliferation (18). In addition, these animals do not undergo significant regeneration after injury (P. Anversa, personal communication). In another study, it has been shown that mice overexpressing a truncated form of human IGF-1 (IGF-1A, which does not containing class 1 or 2 signal peptides) under the control of α-skeletal actin promoter induced physiological and then pathological cardiac hypertrophy, associated with a decreased systolic performance and increased fibrosis (19).

Multiple lines of evidence, demonstrating the capacity of a particular insulin-like growth factor-1 isoform (mIgf-1), expressed locally in adult post-mitotic tissues such as skeletal muscle and heart, to recapitulate the regenerative capacity of prenatal/neonatal tissues, have been generated. The mIGF-1 isoform comprises a Class 1 signal peptide, and an Ea extension peptide. Expression of the mIGF-1 isoform as a transgene in animal skeletal and cardiac muscles resolves inflammation, enhances distal cell survival in a paracrine manner, increases chemoattractive mechanisms and mobilizes circulating bone marrow and endogenous progenitor cells to repair tissue damage. In neonatal tissues, this isoform is expressed at high levels but declines soon after birth in extrahepatic tissues and decreases further during ageing. It has been demonstrated that mIgf-1, delivered as a muscle-specific transgene or virus to mouse skeletal muscle, enhances repair of skeletal muscle damage, enhancement of exercise-induced hypertrophy, reversal of age-related atrophy, and prevention of dystrophic muscle degeneration (20; 21, 22; 23). When expressed as a cardiac-specific transgene, mIGF-1 transiently increased cardiac mass during post-natal stages due to sustained increases in protein translational components and heightened expression of physiological but not pathological markers of cardiac growth and hypertrophy. Induction of myocardial infarction produces localised damage, cell death and massive inflammation but mIGF-1 transgenic animals rapidly resolve in complete repair of the injured heart without scar formation and late-onset proliferation near the site of injury. Down-regulation of specific inflammatory cytokines suggests that mIGF-1 improves cardiac regeneration in part by modulation of the inflammatory response. Since supplementary expression of this growth factor does not alter normal heart development or long-term postnatal cardiac form and function, the enhancement of cardiac regeneration and repair by localised expression of mIGF-1 suggests novel and clinically feasible therapeutic strategies.

Taken together, these data implicate mIGF-1 as a powerful enhancer of the regeneration response, mediating the recruitment of bone marrow and other progenitor cells to sites of tissue damage and augmenting local repair mechanisms. However, the precise mechanism of mIGF-1 action has until now proved elusive.

### SUMMARY OF THE INVENTION

The effect of various C terminal extension or E peptides of IGF-I has been studied in a range of cells and organisms. Whereas the current consensus suggests that it is the mature form of IGF-1 that is responsible for the various physiological effects noted for this protein, the applicant has ascribed many of these effects to the E peptides. Until now, the multiple E peptides generated by alternate splicing have been largely ignored as a potential source of functional diversity.

The invention thus involves the generation and systemic or localized delivery of IGF-1 protein isoforms, including small peptides (35-41 aa) encoding the various sequences of the IGF-1 E peptides to damaged or degenerating tissues. The underlying premise of the invention is that IGF-1 E peptides have unique subsets of function encoded in the full length protein, in particular, the regenerative capacity of IGF-1.

Accordingly, one aspect of the present invention relates to the use of an IGF-I Ea peptide for the regulation of cellular growth or differentiation. The Ea peptide is shown herein to have an important role in the proliferation, differentiation and regeneration of various cell types. For the first time, the inventors have demonstrated that, rather than the mature 70 amino acid IGF-1 peptide, it is the C terminal 35 amino acid Ea peptide that is responsible for some of its functions. Furthermore, various of the physiologically interesting effects of IGF-1 have been assigned by the inventors to the Eb peptide.

This discovery has significant ramifications. A number of applications have been suggested for mIgf-1, in the correction of various neuromuscular and cardiovascular pathologies. Now that it is known that the entity responsible for regulating certain cellular growth functions are the E peptides of IGF-1, previously ignored as a potential source of functional diversity, it is possible to tailor more precisely the application of these peptides in a therapeutic context. In particular, the teaching of the invention may be applied to conditions such as those listed below, by administering E peptides, or other recombinant proteins including E peptides, or nucleic acids encoding such peptide or protein entities, to an affected patient.

### Traumatic Skeletal Muscle Injury

For example, a subset of myogenic progenitors is enhanced in injured mIGF-1 transgenic muscles, expressing the haematopoietic markers CD45, CD11b, c-Kit and Sca-1. Upon muscle injury, these cells increase also in the bone marrow compartment, revealing an unexpected response to distal trauma. Damaged mIGF-1 transgenic muscles activate novel genes implicated in urodele amphibian regeneration. In regenerating MLC/mIGF-1 transgenic muscles, cell populations expressing stem cell and myeloid markers exhibited accelerated myogenic differentiation. In vitro, primary myoblast cultures from MLC/mIGF-1 muscles readily converted co-cultured bone marrow to a myogenic lineage and incorporate bone marrow cells by fusion to muscle fibres (24). The local changes effected by postmitotic expression of the mIGF-1 transgene are illustrated by the enhanced myogenic differentiation of primary stem cell cultures isolated from regenerating mIGF-1 muscles, which unexpectedly re-entered the cell cycle upon serum stimulation, proliferated readily and differentiated upon serum withdrawal. Increased levels of chemokine receptors in mIGF-1 damaged muscle suggest a mechanism whereby circulating cells, which express the corresponding chemokines, are drawn in larger numbers to target organs expressing mIgf-1.

### Muscle Wasting

Metabolic abnormalities in advanced chronic heart failure include functional and morphological decrements in the skeletal musculature that result in progressive muscular atrophy. An experimental model of left ventricular dysfunction was used to detect alterations in the skeletal muscle proteolytic ubiquitin-proteasome pathway, and to assess the potential therapeutic role of supplemental mIGF-1 in attenuating muscle atrophy. Twelve weeks after coronary artery ligation, left ventricular dysfunction and enlargement were observed in wildtype mice and in their transgenic littermates expressing mIGF-1 exclusively in skeletal muscle. Skeletal muscular atrophy in wildtype mice with left ventricular dysfunction was accompanied by an increase in myosin heavy chain ubiquitination, enhanced proteasome activity, and robust induction of Atrogin-1, an ubiquitin-conjugating E3 ligase. In contrast, overexpression of transgenic mIGF-1 prevented muscular atrophy and proteasome activity. The findings suggest that atrophy of the skeletal musculature in mice with left ventricular dysfunction occurs through targeting of specific structural proteins by the ubiquitin-proteasome pathway. The inhibition of muscle atrophy by supplemental mIGF-1 expression provides a promising therapeutic avenue for the prevention of skeletal muscle wasting in chronic heart failure (Schulze et al, manuscript submitted).

### Amyotrophic Lateral Sclerosis

Amyotrophic lateral sclerosis (ALS) is a progressive, lethal neuromuscular disease that is associated with the degeneration of motor neurons, leading to atrophy of limb, axial, and respiratory muscles. Although certain inherited forms of ALS have been attributed to acquired toxic properties associated with a dominant mutation in the SOD1 gene, the aetiology of the disease and the cellular targets critical to the degenerative process have remained difficult to define.

It has been shown that muscle-restricted, hypertrophic action of mIGF-1 maintained muscle integrity and enhanced satellite cell activity in a mutant SOD1 transgenic mouse model of ALS, induced calcineurin-mediated regenerative pathways, and reduced components of catabolic activity. mIGF-1 transgene expression also stabilized neuromuscular junctions enhanced both proximal and distal neuronal survival in mutant SOD1 mice, thus delaying the onset and progression of the disease. These studies establish skeletal muscle as a primary target for the dominant action of inherited SOD1 mutations in motor neuronal degradation. The protection afforded by mIGF-1 action in skeletal muscle in conjunction with the teaching of the present invention suggests novel therapeutic strategies to attenuate the neuronal degeneration associated with ALS (Dobrowolny et al., manuscript in preparation). For example, E peptides, or other recombinant proteins including E peptides, or nucleic acids encoding such peptide entities, may be administered to an affected patient.

### Myocardial Infarction

When delivered as a transgene restricted to the myocardium under the control of the alpha-MHC promoter (α-MHC) to exclude possible endocrine effects on other tissues, an mIGF-1 gene produced accelerated growth during postnatal heart development, but never exceeded wild-type cardiac size in the adult, with a comparative size by 6 months. mIgf1-induced remodelling was accompanied by increased activation of ERK and JNK signalling at one week after birth, and by increased ANP and BNP transcripts at one and two months. Sustained translational activity was observed during all phases of heart development in mIGF-1 overexpressing heart, independent of AKT activation. Early increased heart size of mIGF-1 transgenic hearts was not due to increased cardiomyocyte proliferation, nor did it lead to pathological conditions, as shown by a comparable electrophysiological function between transgenic and wild-type hearts.

The regenerative capacity of mIGF-1 transgenic hearts was analyzed by direct cardiotoxin injection into the heart of four months old mice. Cardiotoxin produced a reproducible and localized damage of the right and left ventricles 48 hours post-injection, in both wild-type and transgenic hearts, with evident cell death and massive inflammation. In contrast to the progression of scar formation in wild-type hearts, transgenic mIGF-1 overexpression induced complete repair of the injured heart after 1 month, without scar formation and with proper tissue reconstitution. Down-regulation of specific inflammatory cytokines suggested that mIGF-1 induced heart regeneration by lowering the inflammatory response. To assess cardiac hyperplasia, cell cycle was assayed by measuring the nuclear incorporation of bromodeoxyuridine (BrdU), a marker of DNA synthesis, 1 month after cardiotoxin injection.

The mIGF-1 transgene induced a significant percentage of total cells to enter cell cycle in response to cardiotoxin injection compared to wild-type hearts. It was found that cardiac cells re-enter the cell cycle, although cells of diverse lineage were found in the myocardium and in the vessels. The nature of these cells is still under investigation. No differences in proliferative state were found in injured heart 24 hour and 1 week after cardiotoxin injection, indicating that the repair program, following the early activation of regeneration program, is activated as a late step in mIGF-1 transgenic hearts.

One aspect of the invention therefore provides an isolated Ea IGF-1 peptide, as defined herein. The invention also provides methods for the regulation of cellular growth or differentiation, comprising exposing a cell to the Ea IGF-1 peptide in a physiologically effective amount.

A further aspect of the invention provides an isolated Eb IGF-1 peptide, as defined herein. The invention also provides methods for the regulation of cellular growth, comprising exposing a cell to the Eb IGF-1 peptide in a physiologically effective amount.

By the “IGF-1 Ea peptide” is meant the 35 amino acid C terminal peptide translated from part of exons 4 and 5 of the IGF-1 gene as part of the IGF-1 propeptide and which is cleaved off during post-translational processing.

By the “IGF-1 Eb peptide” is meant the 41 amino acid C terminal peptide translated from parts of exons 4, 5 and 6 of the IGF-1 gene as part of the IGF-1 propeptide and which is cleaved off during post-translational processing.

Preferably, the IGF-1 Ea peptide and IGF-1 Eb peptide are of human origin.

By “regulation of cellular growth” is meant that the peptide entity is capable of altering, preferably increasing cellular growth. Said cells may be singular or form part of a cellular mass, such as a tissue or organ. In particular, the Ea peptide may cause hypertrophy of muscle tissue or adipose tissue. Examples of muscle tissue will be known to the person of skill in the art and include skeletal (striated), smooth and cardiac muscle tissue.

By “differentiation” is meant that the peptide entity is capable of inducing biochemical and structural changes in an unspecialized cell, thereby causing its form and function to become specialised. Examples of such differentiation include repair of diseased (including cancerous) cells, alteration of the genetic constitution of cells, induction of specific cell types and cell fates, changing the immunological profiles of cells, and inducing particular desired immune functions or properties. The alteration of the property may result in the cell undergoing differentiation towards a more specialised form or function, for example from a stem cell towards an adult cell with a specialised function (for example circulating bone marrow-derived cells such as myeloid progenitors).

The peptide can work either as an isolated peptide or as a fusion with another entity. The peptide of the invention will typically be a polypeptide e.g. consisting of between 20 and 500 amino acids. The polypeptide preferably consists of no more than 200 amino acids (e.g. no more than 190, 180, 170, 160, 150, 140, 130, 120, 110, 100, 90, 80, 70, 60 or no more than 50). Details of particular preferred polypeptides for use in accordance with the invention are given below.

The peptides of the invention may be used to regulate cellular growth or differentiation in any cell type, for example, muscle tissue; nervous tissue; adipose tissue; cartilage; bone; hepatic tissue; kidney tissue; hair; or skin.

The peptides of the invention may be used in the prevention and treatment of cellular trauma. The trauma may be to any cell type and caused as a result of any form of trauma. Particular examples include crush injury; surgical damage; muscle tear injury; nerve damage; surgical damage; myocardial infarction; stroke; ischemia; burns; bone fractures or UV damage.

Although the Applicant does not wish to be limited or bound by any particular theory, it is postulated herein that the E peptide is cleaved from the core IGF-I molecule by thrombin. The putative cleavage site that separates the variable E peptides, to generate the mature 70 amino acid IGF-1 protein, has been characterised. Unexpectedly, this consensus sequence, highly conserved between species, corresponds to a thrombin cleavage site, raising the exciting possibility that the clotting cascade may act as a stimulus for regenerative action of the IGF-1 precursor via the release of E peptides. In this model, the activation of prothrombin to thrombin, as a result of cellular trauma, results in the cleavage of fibrinogen to fibrin and the subsequent formation of blood clots. It is thus postulated that the mature 70 amino acid IGF-I peptide and E peptides circulate in the body fused together as an inactive pro-form of the IGF-1 protein. Once activated as a result of trauma, thrombin then cleaves, and as a consequence activates the various E peptides from the IGF-I proprotein. In this model, this cleavage and activation of the IGF-I E peptides will occur only at sites of localised cellular trauma, providing an activation signal that is economic, in the sense that it is not needlessly wasteful, and localised, in the sense that it is specific to a site where regenerative capacity is required.

As a consequence of the Applicant's theory, the invention also provides for inactive pro-forms of the IGF-1 Ea and Eb peptides which are activatable by thrombin cleavage. Such peptides of the invention may be administered prior to trauma in an attempt to prevent and reduce the damage. For example, in the case of patients at high risk of a condition such as myocardial infarction, peptides may be administered to achieve systemic circulating levels of a proprotein form of an IGF-1 E peptide, thus allowing for a faster response to localised cellular trauma if and when this occurs.

Peptides of the invention may also be administered to patients awaiting surgery. Heightened systemic levels of the IGF-1 Ea pro-form would provide a patient with an enhanced ability to deal with cellular trauma caused by surgery.

Peptides of the invention may also be administered shortly after trauma. An example is provided by the case of a patient having suffered internal injuries. Administration of the peptides of the invention will provide the patient with a heightened ability to deal with such injuries. Internal injuries may be as a result of a physical trauma, such as a vehicle accident, or may be from a pre-existing condition such as a myocardial infarction.

Peptides of the invention may be delivered locally or systemically to enhance the regeneration of injured or degenerating tissue.

Peptides of the invention can also be used to treat external injuries by topical application. One example is provided by a patient suffering wounds to the skin. Such wounds could be caused by a variety of traumas including laceration and burns.

Peptides of the invention can also be topically, co-administered with thrombin. For example, peptides of the invention may be administered during surgical procedures, along with thrombin, to aid with hemostasis.

Peptides of the invention can also be used in the prevention and treatment of muscular atrophy and related conditions. Such muscular atrophy may be as a result of the ageing process (sarcopenia). Muscle weakening and frailty are well documented effects of the ageing process. Peptides of the invention may be administered as a preventative measure, that is, as a regular supplement to slow the muscular atrophy caused by the ageing process. They may also be administered at a later stage to both slow and reverse the age related affects of muscular atrophy.

Such muscular atrophy may be caused by neuromuscular or neurodegenerative disorders. These disorders may be acquired or hereditary and can include Parkinson's Disease, including early onset forms (Autosomal recessive juvenile Parkinson's; ARJP), Lewy body dementias, and general synucleinopathies; Alzheimer's disease, including frontotemporal dementias (FTD), corticobasal degeneration (CBD), progressive supranuclear palsy (PSP), and general tauopathies and amyloidopathies; Amyotrophic Lateral Sclerosis, including adult-onset motor neuron disease; Huntington's disease, including spino-cerebellar ataxias and adult onset trinucleotide repeat disorders.

Such muscular atrophy may be caused by muscular dystrophys; such conditions can include Becker muscular dystrophy, distal muscular dystrophy, Duchenne's muscular dystrophy, limb-girdle muscular dystrophy, myotonic dystrophy and oculopharyngeal muscular dystrophy.

Moreover, said muscular atrophy may be induced by congestive heart failure, cardiomyopathies, atherosclerosis, acute insult including myocarditis or myocardial infarction.

In a further aspect, the peptides of the invention may be used to treat muscular atrophy caused by the disuse of a muscle. This disuse may be caused by a spinal chord injury or through the immobilisation of a limb through injury, for example, resulting from correction of a bone fracture. Said disuse may also be caused as result of a patient suffering from a stroke.

Peptides of the invention may also be used for increasing muscle hypertrophy. Muscle hypertrophy may be increased as an aid to physical therapy. An example is provided by helping a patient gain weight after a severe illness, injury, or continuing infection. Peptides of the invention may also be used to increase muscle hypertrophy in livestock in order to increase yields. Examples of clear commercial relevance include cattle, sheep, pigs and fish. Other examples may be found in sports medicine, or in body-building.

Peptides of the invention may also be used for increasing adipose tissue deposits. Ea peptides, and fusion proteins including these peptides, will be of particular use in this context. Adipose tissue may be increased to help a patient gain weight after a severe illness, injury, or continuing infection. Peptides of the invention may also be used to aid in the treatment of Anorexia nervosa or Bulimia nervosa.

Although the Applicant does not wish to be limited or bound by any particular theory, it is postulated herein that mIGF-1 accelerates the timing of regeneration and reduces the amount of mononucleated infiltrating cells post-injury. The local expression of mIGF-1 improves the regenerative phase increasing the pool of satellite cells and modulating the inflammatory response of injured skeletal muscle. Furthermore, mIGF-1 modulates inflammatory cytokines, such as MCP1, MCP2, MIP-1α, and MIP-1β at early stages, stimulating a qualitative environment for complete functional recovery. It is proposed that mIGF-1 modulates inflammatory cytokines at early stages, stimulating a qualitative environment for a complete functional recovery.

As a consequence of the Applicant's theory, the invention also provides for use of the peptides of the invention for improving the dystrophic environment and/or stimulating the regenerative capacity of stem cells. Ea peptides, and fusion proteins including these peptides, will be of particular use in this context.

A peptide of the invention may have the formula NH₂-A-B—C-D-COOH, wherein: -A- is an optional N-terminus amino acid sequence consisting of a amino acids; —B— is an optional amino acid sequence consisting of b amino acids; —C— is a sequence derived from an IGF-1 Ea or Eb peptide; and -D- is an optional C-terminus amino acid sequence consisting of d amino acids. The positions of entities B and C relative to each other may be reversed in the protein sequence, if necessary.

As defined above, -A- is an optional N-terminus amino acid sequence consisting of a amino acids. The value of a is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500, etc.), but can be zero (i.e.-A- is absent). Examples of typical -A- moieties include leader sequences to direct protein trafficking, or short peptide sequences which facilitate cloning or purification (e.g. histidine tags i.e. Hisₙ where n=3, 4, 5, 6, 7, 8, 9, 10 or more). In some embodiments, moiety -A- is, or terminates at its N-terminus with, a methionine residue. Other examples of -A- moieties include IGF-1 signal peptides, such as the Class 1 IGF-1 signal peptide, consisting of 48 amino acid residues derived from parts of exons 1 and 3 of the IGF-1 gene; the Class 2 IGF-1 signal peptide, consisting of 32 amino acid residues derived from parts of exons 2 and 3 of the IGF-1 gene; and the Class 3 IGF-1 signal peptide, consisting of 22 amino acid residues derived from exon 3 of the IGF-1 gene. Other suitable N-terminus amino acid sequences will be apparent to those skilled in the art.

As defined above, —B— is an optional amino acid sequence consisting of b amino acids. The value of b is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500, etc.), but can be zero (i.e. —B— is absent). One example of a suitable entity for B includes the mature 70 amino acid IGF-1 peptide derived from parts of exons 3 and 4 of the IGF-1 gene. For example, a fusion peptide including the mature IGF-1 form lined either to the Ea or Eb IGF-1 peptide might be used to act as a biologically inactive propeptide that is cleaved when required, thereby to elicit its cell regulating effects.

As defined above, —C— is a sequence derived from an IGF-1 Ea or Eb peptide. The function of —C— is to act as a regulator of cellular growth, as set out above. In instances where —C— is the isolated IGF-1 Ea or Eb peptide, the peptide will be constitutively active as a regulator of cellular growth. Alternatively, when expressed as a fusion with other peptide entities, by tailoring the identity of the fusion partner, various effects can be achieved. For example, when tethered to the mature 70 amino acid IGF-1 peptide, the fusion will act as a kind of proprotein, which can be administered systemically to the circulation of a patient to provide function when and where this is required.

In some embodiments, the amino acid sequence of —C— shares less than x % sequence identity to the b amino acids which are N-terminal of sequence —C— in the specific protein from which —C— is derived. In general, the value of x is 60 or less (e.g. 50, 40, 30, 20, 10 or less).

As defined above, -D- is an optional C-terminus amino acid sequence consisting of d amino acids. The value of d is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500, etc.), but can be zero (i.e.-D- is absent). Examples of typical -D- moieties include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminus amino acid sequences will be apparent to those skilled in the art. In certain embodiments, the function of -D- is to facilitate expression of the protein in an expression system.

The value of a+d may be 0 or greater (e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500 etc.). It is preferred that the value of a+d is at most 1000 (e.g. at most 900, 800, 700, 600, 500, 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9, 8, 7, 6, 5, 4, 3, 2).

Preferably, component —B—C— of the above-noted formula comprises a fusion of the mature 70 amino acid IGF-1 peptide with the IGF-1 Ea or Eb peptide (SEQ ID NO:2 or SEQ ID NO:4), or is a functional equivalent thereof.

In some polypeptides, the amino acid sequences of the -A-, —B—, —C— and -D- moieties may contain m amino acid substitutions, where m is an integer. The m amino acids are typically substituted by A, C, D, E, F, G, H, I, K, L, M, N, P, Q, R, S, T, V, W, or Y. Each of the m substitutions may be the same or different as the others. The substitution is preferably by G or, more preferably, by A. The substituting amino acid may be an L or a D amino acid but, where the other amino acids all share a single stereo-configuration (i.e. all D or all L), the substituting amino acid preferably also has that stereo-configuration (although, of course, G has no stereoisomers).

The invention also provides a peptide, comprising amino acid sequence -A-B—C-D-, wherein: -A- is an optional methionine residue; —B— is an optional amino acid sequence with at least a % sequence identity to SEQ ID NO:12; and —C— is an amino acid sequence with at least b % sequence identity to SEQ ID NO:2 or SEQ ID NO:4; -D- is an optional amino acid sequence.

The value of a is 50 or more. The value of b is 50 or more. The value of c is 50 or more. The value of d is 50 or more. The values of a, b, c and d are independent of each other, and typical values are 60, 70, 80, 90, 95, 96, 97, 98, 99 or 100. Preferably, the value of d is 100.

Preferably, the peptide comprises SEQ ID NO:2, SEQ ID NO:4, SEQ ID NO:6, SEQ ID NO:8, SEQ ID NO:10 and/or SEQ ID NO:12, or is a functional equivalent thereof. More preferably, the peptide consists of SEQ ID NO:2 and/or SEQ ID NO:4, or is a functional equivalent thereof.

The present invention also provides truncations of the peptides of the invention. For example, the N-terminus may be truncated by at least 1, 2, 3, 4, 5, 6, 7, 8, 9, 10, 11, 12, 13, 14, 15, 16, 17, 18, 19 20 or more.

Peptides of the invention (including oligopeptides and polypeptides, collectively “peptides”) may be linear, branched or cyclic, but they are preferably linear chains of amino acids. Where cysteine residues are present, peptides of the invention may be linked to other peptides via disulphide bridges. Peptides of the invention may comprise L-amino acids and/or D-amino acids. The inclusion of D-amino acids may be preferred in order to confer resistance to mammalian proteases.

The N-terminus residue of a peptide of the invention may be covalently modified. Suitable covalent groups include, but are not limited to: acetyl (as in Fuzeon™); a hydrophobic group; carbobenzoxyl; dansyl; T-butyloxycarbonyl; amido; 9-fluorenylmethoxy-carbonyl (FMOC); a lipid; a fatty acid; polyethylene; carbohydrate; etc.

Similarly, the C-terminus residue of a peptide may be covalently modified (e.g. carboxamide, as in Fuzeon™, etc.). Suitable covalent groups include, but are not limited to: acetyl; a hydrophobic group; amido; carbobenzoxyl; dansyl; T-butyloxycarbonyl; 9-fluorenylmethoxy-carbonyl (FMOC); a lipid; a fatty acid; polyethylene; carbohydrate; etc.

Peptides of the invention may be produced by various means.

A preferred method for production is biological synthesis, e.g. the peptides may be produced by translation. This may be carried out in vitro or in vivo. Biological methods are in general restricted to the production of peptides based on L-amino acids, but manipulation of translation machinery (e.g. of aminoacyl-tRNA molecules) can be used to allow the introduction of D-amino acids (or of other non-natural amino acids, such as iodotyrosine or methylphenylalanine, azidohomoalanine, etc.) {25}.

Production of peptides by biological means gives peptides with an N-terminus methionine residue. Where the N-terminus of a peptide of the invention is not a methionine then this residue (and any other extraneous residues) will have to be removed e.g. by proteolytic digestion.

The invention also provides a purified nucleic acid molecule which encodes a polypeptide according to any of the above embodiments of the invention.

Preferably, the purified nucleic acid molecule comprises the nucleic acid sequence as recited in SEQ ID NO:1 (encoding the Human Ea peptide protein sequence), SEQ ID NO:3 (encoding the Human Eb peptide protein sequence), SEQ ID NO:5 (encoding the Human Class 1 IGF-1 signal peptide protein sequence), SEQ ID NO:7 (encoding the Human Class 2 IGF-1 signal peptide protein sequence), SEQ ID NO:9 (encoding the Human Class 3 IGF-1 signal peptide protein sequence), SEQ ID NO:11 (encoding the Human mature IGF-1 peptide protein sequence) or is a redundant equivalent or fragment of any one of these sequences.

The invention further provides that the purified nucleic acid molecule consists of the nucleic acid sequences as recited in SEQ ID NO:1 (encoding the Human Ea peptide protein sequence), SEQ ID NO:3 (encoding the Human Eb peptide protein sequence), SEQ ID NO:5 (encoding the Human Class 1 IGF-1 signal peptide protein sequence), SEQ ID NO:7 (encoding the Human Class 2 IGF-1 signal peptide protein sequence), SEQ ID NO:9 (encoding the Human Class 3 IGF-1 signal peptide protein sequence), SEQ ID NO:11 encoding the Human mature IGF-1 peptide protein sequence) or is a redundant equivalent or fragment of any one of these sequences.

The nucleic acid may be DNA or RNA (or hybrids thereof), or their analogues, such as those containing modified backbones (e.g. phosphorothioates) or peptide nucleic acids (PNA). It may be single-stranded (e.g. mRNA) or double-stranded, and the invention includes both individual strands of a double-stranded nucleic acid (e.g. for antisense, priming or probing purposes). It may be linear or circular. It may be labelled. It may be attached to a solid support.

Nucleic acid according to the invention can, of course, be prepared in many ways e.g. by chemical synthesis (e.g. phosphoramidite synthesis of DNA) in whole or in part, by nuclease digestion of longer molecules, by ligation of shorter molecules, from genomic or cDNA libraries, by use of polymerases etc.

Accordingly, the present invention also provides vectors (e.g. plasmids) comprising nucleic acid of the invention (e.g. expression vectors and cloning vectors) and host cells (prokaryotic or eukaryotic) transformed with such vectors.

The invention also provides a process for producing a peptide of the invention, comprising the step of culturing a host cell transformed with nucleic acid of the invention under conditions that induce expression of the peptide.

Suitable expression systems for use in the present invention are well known to those of skill in the art and many are described in detail in references 26 and 27.

Generally, any system or vector that is suitable to maintain, propagate or express nucleic acid molecules to produce a peptide in the required host may be used. The appropriate nucleotide sequence may be inserted into an expression system by any of a variety of well-known and routine techniques, such as, for example, those described in 26.

Generally, the encoding gene can be placed under the control of a control element such as a promoter, ribosome binding site (for bacterial expression) and, optionally, an operator, so that the DNA sequence encoding the desired peptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the peptides of the invention.

For long-term, high-yield production of a recombinant peptide, stable expression is preferred. For example, cell lines that stably express the peptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in reference 28. Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in references 29, 30, 31 and 32. In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred prokaryotic expression systems include those that use streptococci, staphylococci, E. coli, Streptomyces and Bacillus subtilis as host cells. Examples of particularly suitable fungal expression systems include those that use yeast (for example, S. cerevisiae) and Aspergillus as host cells.

An alternative to biological synthesis for producing peptides of the invention involves in vitro chemical synthesis {33, 34}. Solid-phase peptide synthesis is particularly preferred, such as methods based on t-Boc or Fmoc {35} chemistry. Enzymatic synthesis {36} may also be used in part or in full. Where D-amino acids are included in peptides of the invention it is preferred to use chemical synthesis.

Accordingly, the invention also provides a process for producing a peptide of the invention, comprising the step of synthesising the peptide by chemical means. The peptide may be synthesised in whole or in part by such chemical means.

Peptides of the invention are useful regulators of cellular growth in their own right. However, they may be refined to improve this regulatory activity or to improve pharmacologically important features such as bioavailability, toxicology, metabolism, pharmacokinetics, etc. The peptides may therefore be used as lead compounds for further research and refinement.

The invention provides a pharmaceutical composition comprising (a) a peptide of the invention and (b) a pharmaceutical carrier.

Component (a) is the active ingredient in the composition, and this is present at a therapeutically effective amount e.g. an amount sufficient to inhibit infection. The precise effective amount for a given patient will depend upon their size and health, the nature and extent of infection, and the composition or combination of compositions selected for administration. The effective amount can be determined by routine experimentation and is within the judgement of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg. Pharmaceutical compositions based on peptides are well known in the art (e.g. FUZEON™). Peptides may be included in the composition in the form of salts and/or esters.

Carrier (b) can be any substance that does not itself induce the production of antibodies harmful to the patient receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers can include liquids such as water, saline, glycerol and ethanol. Auxiliary substances, such as wetting or emulsifying agents, pH buffering substances, and the like, can also be present in such vehicles. Liposomes are suitable carriers. A thorough discussion of pharmaceutical carriers is available in reference 37.

The carriers may be liposomes. “Liposome” refers to a generally spherical cluster or aggregate of amphipathic compounds, including lipid compounds, typically in the form of one or more concentric layers, for example, monolayers and/or bilayers. The liposomes may be formulated, for example, from ionic lipids and/or non-ionic lipids. The preparation of suitable liposomes would be well known to those of skill in the art (see, for example, reference 38). The peptide may be incorporated in the liposome in a variety of ways. Generally speaking, the peptide may be incorporated by being associated covalently or non-covalently with one or more of the materials which are included in the liposomes. In a preferred embodiment, the peptide is incorporated in the liposome via non-covalent associations. As known to those skilled in the art, non-covalent association is generally a function of a variety of factors, including, for example, the polarity of the involved molecules and the charge (positive or negative), if any, of the involved molecules, and the like. Non-covalent bonds are preferably selected from the group consisting of ionic interaction, dipole-dipole interaction, hydrogen bonds, hydrophilic interactions, van der Waal's forces, and any combinations thereof. Preferably, the peptide is incorporated in the liposome by means of a transmembrane domain that forms part of the peptide. Preferably, the peptide is incorporated in the liposome such that sequence derived from an HR2 domain is on the outside face of the liposome.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared. The composition may be prepared for topical administration e.g. as an ointment, gel, cream or powder. The composition may be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops, as a spray, or as a powder (as described in reference 39). The composition may be lyophilised.

The pharmaceutical composition is preferably sterile. It is preferably pyrogen-free. It is preferably buffered e.g. at between pH 6 and pH 8, generally around pH 7.

The invention also provides a delivery device containing a pharmaceutical composition of the invention. The device may be, for example, a syringe or an inhaler.

The invention provides a peptide of the invention for use as a medicament. The invention also provides a method for treating a subject suffering from or at risk of contracting a disease or medical condition, comprising administering to the subject a pharmaceutical composition of the invention. The invention also provides the use of a pharmaceutical composition of the invention in the manufacture of a medicament for treating a subject.

Particular conditions that may be treated by the pharmaceutical compositions of the present invention include traumatic skeletal muscle injury, muscle wasting, amyotrophic lateral sclerosis and myocardial infarction.

The subject is preferably a mammal, more preferably a human. The human may be an adult or a child. A composition intended for children may also be administered to adults e.g. to assess safety, dosage, immunogenicity, etc.

Compositions of the invention will generally be administered directly to a subject. Direct delivery may be accomplished by parenteral injection (e.g. subcutaneously, intraperitoneally, intravenously, intramuscularly, or to the interstitial space of a tissue), or by rectal, oral (e.g. tablet, spray), vaginal, topical, transdermal or transcutaneous, intranasal, pulmonary or other mucosal administration.

Dosage treatment can be a single dose schedule or a multiple dose schedule.

Gene therapy may be employed to effect the endogenous production of the peptide by the relevant cells in the subject. Gene therapy is used to treat permanently the inappropriate production of the peptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

Ex vivo gene therapy may also involve the isolation and purification of adult stem cells, the introduction of a gene encoding a peptide of the invention and introduction of the genetically altered adult stem cells into the patient. For example, circulating bone-marrow derived cells may be used to target a tissue restricted gene expression cassette, encoding an E peptide, to damaged, inflamed or degenerating tissues. More specifically myeloid progenitor cells may be used for direct muscle delivery. These cells migrate to sites of injury as a normal component of the inflammation response, where they differentiate into a number of cell types, including macrophages which increase muscle satellite cell proliferation and enhance regeneration (40). Myeloid progenitors have recently been shown to fuse directly with differentiated skeletal muscle fibres and transdifferentiate along a myogenic lineage (41). Endogenous myeloid cell fusion is normally a rare event, however once incorporated into the damaged muscle bed, activation of the E peptide gene in the new fused myeloid nuclei will enhance subsequent recruitment and incorporation of additional circulating cells, thereby augmenting the overall incorporation of the E peptide gene-carrying myeloid cells over time. Repeated administration of engineered myeloid cells carrying the E peptide gene may optimise gene uptake in chronic degenerative conditions.

The therapeutic gene is typically “packaged” for administration to a patient. Gene delivery vehicles may be non-viral, such as liposomes, or replication-deficient viruses, such as adenovirus as described by Berkner (42) or adeno-associated virus (AAV) vectors as described by Muzyczka, (43; 44). For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the peptide, such that the packaging cell now produces infectious viral particles containing the gene of interest. These producer cells may be administered to a subject for engineering cells in vivo and expression of the peptide in vivo (45).

Another approach is the administration of “naked DNA” in which the therapeutic gene is directly injected into the bloodstream or muscle tissue.

The uses and methods of the invention can be used therapeutically (e.g. for treating an existing infections) or prophylactically (e.g. in a situation where disease is expected and where establishment of disease is to be prevented). Therapeutic use is preferred, and efficacy of treatment can be tested by monitoring the patient after administration of the pharmaceutical composition of the invention, such as by monitoring symptoms.

The invention also includes the use of a peptide according to any of the above-described aspects of the invention, or a functional equivalent thereof, a nucleic acid molecule encoding said peptide or functional equivalent, in the manufacture of a medicament for increasing muscle hypertrophy.

The invention also includes the use of a peptide according to any of the above-described aspects of the invention, or a functional equivalent thereof, a nucleic acid molecule encoding said peptide or functional equivalent, in the manufacture of a medicament for decreasing muscle atrophy.

The invention also includes the use of a peptide according to any of the above-described aspects of the invention, or a functional equivalent thereof, a nucleic acid molecule encoding said peptide or functional equivalent, in the manufacture of a medicament for attenuation of neuronal degeneration.

The term “comprising” encompasses “including” as well as “consisting of” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

The term “functional equivalent”, as used herein, refers to a sequence that has an analogous function to the sequence of which it is a functional equivalent. By “analogous function” is meant that the sequences share a common function, for example, in the regulation of cellular growth or differentiation, and, in some embodiments, a common evolutionary origin. In some embodiments, a functionally equivalent sequence may exhibit sequence identity with the sequence of which it is a functional equivalent. Preferably, the sequence identity between the functional equivalent and the sequence of which it is a functional equivalent is at least 50% across the length of the functional equivalent. More preferably, the identity is at least 60% across the length of the functional equivalent. Even more preferably, identity is greater than 70%, 80%, 85%, 90%, 95%, 96%, 97%, 98% or 99% across the length of the functional equivalent. Functional equivalents include mutants of the sequences of which they are functional equivalents, i.e. containing amino acid substitutions, insertions or deletions from said sequence, provided that function is retained. Functional equivalents with improved function compared to the sequences of which they are functional equivalents may be designed through the systematic or directed mutation of specific residues in said sequences. Functional equivalents include sequences containing conservative amino acid substitutions that do not affect the function or activity of the sequence in an adverse manner.

References to a percentage sequence identity between two amino acid sequences means that, when aligned, a percentage of the amino acids are the same in the two sequences. This alignment and the percent homology or sequence identity can be determined using software programs known in the art, for example those described in section 7.7.18 of reference 46. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in reference 47.

The use of “NH₂” and “COOH” in peptide sequences implies only the direction of the peptide chain from N-terminus to C-terminus, and does not imply that the N-terminus residue must have a free —NH₂ group or that the C-terminus must have a free —COOH group (although nor is such a situation excluded). On the contrary, the N- and C-termini may be covalently modified.

Preferably, the IGF-1 Ea peptide of the invention will be glycosylated. However, it may not be glycosylated or partially glycosylated in some embodiments. For example, when expressed so as to include the Class 1 signal peptide, the Ea peptide is glycosylated. When expressed so as to include the Class 2 signal peptide, the Ea peptide is not glycosylated.

Although the Applicant does not wish to be limited or bound by any particular theory, it is postulated herein that the distribution pattern of the IGF-1 E peptide can be altered by the class of signal peptide encoded by the nucleic acid encoding a peptide of the invention. For example, when expressed so as to include the Class 1 signal peptide, the IGF-1 E peptide has been shown to act in a paracrine manner. In contrast, when expressed so as to include the Class 2 signal peptide, the IGF-1 E peptide has been shown to act in an endocrine manner. As a consequence of the Applicant's theory, the invention also provides for IGF-1 E peptides, comprising the class 1, class 2 or class 3 signal peptide, specifically designed for either localised or systemic delivery. These signal peptides may be used to direct the delivery of a peptide of the invention in any of the forms described above. For example, situations can be envisaged wherein the desired response, according to any aspect of the invention, can be achieved through the delivery of an E peptide of the invention to a particular cell or tissue type. Alternatively, it may be necessary to deliver an E peptide in conjunction with the mature IGF-1 peptide to achieve the desired response. Further still, a peptide of the invention may be delivered under the control of the various signal peptides, in conjunction with other useful peptides, for example, human growth hormone, optionally linked as a multipeptide unit.

The invention also includes a transgenic animal comprising a nucleic acid encoding a peptide of the invention or a functional equivalent thereof or a fusion protein as defined above. Such transgenic animals may in particular include sheep, pigs, cows, chickens, goats and fish. Particular commercial utilities of such animals may be evident from an increased size, or an increased edible volume, provided by such animals.

Various aspects and embodiments of the present invention will now be described in more detail by way of example. It will be appreciated that modification of detail may be made without departing from the scope of the invention.

### BRIEF DESCRIPTION OF FIGURES

FIG. 1. Muscle Atrophy and Increased Activity of the Proteasome in Mice with Left Ventricular Dysfunction. A, Skeletal muscle atrophy develops in mice with CLVD. B, Increased protein ubiquitination in skeletal muscle of mice with CLVD. C, Activity of the 20S proteasome increases in several atrophying muscles in chronic CLVD (all P<0.05 vs. WT sham) (EDL —M. extensor digitorum longus; SOL—M. soleus; QUA—M. quadriceps; n=6 animals per datapoint).

FIG. 2. Transgenic Overexpression of mIGF-1 Prevents Muscle Atrophy and Increased Proteasome Activity in Left Ventricular Dysfunction. A, Transgenic overexpression of MLC/mIGF-1 prevented the development of muscular atrophy in animals with CLVD. B, Increased protein ubiquitination in skeletal muscle of mice with CLVD is absent in MLC/mIGF-1 mice. C, Overexpression of mIGF-1 blocks enhanced 20S proteasome activity in chronic CLVD (EDL—M. extensor digitorum longus; SOL—M. soleus; QUA—M. quadriceps; n=6 animals per datapoint).

FIG. 3. Expression of atrogin-1/MAFbx. A, Expression of atrogin-1/MAFbx increases in several muscles of animals with CLVD. B, Quantitative real-time PCR revealed increased of atrogin-1/MAFbx expression in the atrophying quadriceps muscle of animals with CLVD (*P<0.05 vs. WT sham; n=6 animals per datapoint) which is prevented by transgenic overexpression of mIgf-1. C, In SOL8 cells, serum starvation for 72 h increases atrogin-1/MAFbx expression which is reduced by serum incubation and stimulation with IGF-1 for 3 h (data from 3 independent experiments).

FIG. 4. Ubiquitin-mediated Proteolysis of MyHC. A, Analysis of MyHC ubiquitination in vitro. While Western analysis shows no differences in overall protein levels between dexamethasone-treated (10 ng/ml for 24 h) cells and controls, ubiquitination of MyHC is robustly enhanced under dexamethasone compared to controls. B, Increased ubiquitination of MyHC in atrophying skeletal muscle 12 weeks after coronary artery ligation and development of CLVD. Expression of the MLC/mIGF-1 transgene prevents the increase in ubiquitination of MyHC in skeletal muscles of animals with CLVD.

FIG. 5. Reduced of Akt Phosphorylation and FOXO Activation in Atrophying Skeletal Muscle is Prevented in MLC/mIGF-1 mice. A, Skeletal muscle of mice with CLVD shows reduced Akt activity compared to controls while overexpression of MLC/mIGF-1 enhances Akt activity without changes in animals with CLVD (n=3 per datapoint). B, Increased activation of FOXO 4 in skeletal muscle of mice with CLVD compared to controls. Transgenic overexpression of mIGF-1 blocks activation of FOXO transcription factors by enhancing FOXO phosphorylation (n=3 per datapoint).

FIG. 6. Characterization of MHC/mIGF-1 transgenic mice. (A) Schematic representation of the rodent Igf1 gene. (B, C) Northern blot analysis of total RNA (10 μg) from different aged wild type and transgenic hearts, using the rat IGF-1 32P-labeled probe. Ethidium Bromide was used to verify equal RNA loading amount and RNA integrity.

FIG. 7. Physiological analysis of mIGF-1 transgenic hearts. (A) Histological analysis of wild-type and transgenic hearts by Hematoxylin and Eosin staining. The relative increase in heart weight/body weight (p-value<0.05) of transgenic hearts is resolved by six months. Values are the average of six independent analyses. (B) RT-PCR of different hypertrophic markers in adult hearts. 0.5 μg of total RNA was used for each single PCR. PCR values were normalized for actin content. (C) Western blot analysis of AKT and S6 ribosomal protein phosphorylation. 50 μg of total cell extracts at different age of postnatal heart development were loaded onto SDS-PAGE gel. Total amount of proteins was normalized for AKT and S6 ribosomal protein.

FIG. 8. Representative electrocardiograms obtained in non-transgenic (NTG) and IGF-1 transgenic (TG) mice in the D2 derivation. The plain arrows indicate the P waves that are amplified in the transgenic mice. The dotted arrows indicate a prolongation and non-homogenous depolarization of the ventricles in the TG mouse.

FIG. 9. Full cardiac regeneration in mIGF-1 transgenic mice. (A, B and C) Thricrome staining of 4 months old wild-type and transgenic hearts at 48 hours, 1 week and 1 month after CTX injection in left ventricular wall. Comparable results were obtained with similar analyses on six different groups of animals

FIG. 10. Early events characterizing mIGF-1 induced regeneration. (A) RT-PCR of inflammatory interleukins IL6 and IL1β 24 hours after cardiotoxin injection in wild-type and transgenic hearts. PCR was normalized by actin content in each sample. (B) Real time PCR of the anti-inflammatory cytokines IL10 and IL4 in transgenic (gray bars) and wild-type (white bars) hearts 24 hour and 1 week after cardiotoxin injection. The results are the average of three independent experiments. (C) Expression of p21WAF1/CIP1 in wild-type and transgenic hearts injected with cardiotoxin. Actin was used to verify equal protein loading amount.

FIG. 11. Cells proliferation accompanies mIGF-1 induced heart regeneration. BrdU was provided ad libitum for 1 month after cardiotoxin injection at 0.1%. Paraffin sections of 10 μm were stained with a byotinalated mouse monoclonal antibody to visualized nuclei that incorporated BrdU in wild type (A) and transgenic (B) hearts. All the sections where the injury was evident were analysed. Cardiac cells (C) and cells of other lineage (D) were observed around the injury.

FIG. 12. mIGF-1 expression delays the progression of the disease and enhances the survival of SOD1_{G93A} mutant mice (a) Western blot analysis for human SOD transgenic protein in wild type (lane 1), MLC/mIGF-1 (lane 2), SOD1_{G93A} (lanes 3) and SOD_{G93A}mIGF-1 (lanes 4) transgenic mice. (b) Northern blot analysis for mIGF-1 transgene expression in skeletal muscle of wild type (lane 1), MLC/mIGF-1 (lane 2), SOD1_{G93A} (lanes 3) and SOD_{G93A}mIGF-1 (lanes 4) transgenic mice; in brain of MLC/mIGF-1 (lane 5), SOD_{G93A}mIGF-1 (lanes 6) mice; in spinal cord of MLC/mIGF-1 (lane 5), SOD_{G93A}mIGF-1 (lanes 6) mice. (c) Age of onset of disease symptoms in SOD1_{G93A} and SOD_{G93A}mIGF-1 mice. (d) Analysis of the progression of the disease in SOD1_{G93A} and SOD_{G93A}mIGF-1 mice. (e) Survival analysis of SOD1_{G93A} and SOD_{G93A}mIGF-1 mice.

FIG. 13. mIGF-1 expression attenuates muscle wasting and promotes regenerative pathways in SOD1_{G93A} mice. (a) Muscle histological analysis of wild type (A), MLC/mIGF-1 (B), SOD1_{G93A} (C, E) and SOD_{G93A}mIGF-1 (D, F) mice at different age and stage of disease. (b) Analysis of fiber type size differences in the quadriceps underscores the relative attenuation of muscle atrophy in SOD_{G93A}mIGF-1 compared to SOD1_{G93A} mice. (c) Western blot analysis for molecular markers of muscle regeneration, activated satellite cells, and maturation (Pax 7, desmin, myogenin, and neo-MyHC). Muscle protein lysates were obtained from quadriceps of wild type (lane 1), MLC/mIGF-1 (lane 2), SOD1_{G93A} (lanes 3, 5) and SOD_{G93A}mIGF-1 (lanes 4, 6) transgenic mice at different ages of the clinical disease (lanes 3 and 4 at 28 days of age; lanes 5 and 6 at 123 days of age). Immunoblotting for α-tubulin served as a control for protein loading. (c) Immunofluorescence analysis for MyHC-fast performed on Soleus muscles of wild type, SOD1_{G93A} and SOD_{G93A}mIGF-1 before (80 days) and after symptom onset (123, 138 days of age). Bar, 50 μm. (d) Walk test of SOD1_{G93A} (black circles) and SOD_{G93A}mIGF-1 (white circles) transgenic mice. The expression of mIGF-1 maintained the functional performance of SOD1_{G93A} skeletal muscle.

FIG. 14. Transgenic mIGF-1 expression induces chronic CnA-β1 expression in SOD1_{G93A} mice. (a) Northern blot analysis for the CnA-β1 of wild type (lane 1), MLC/mIGF-1 (lane 2), SOD1_{G93A} (lane 3), and SOD_{G93A}mIGF-1 (lane 4) transgenic mice. Ethidium bromide staining was used to verify equal loading of the RNA sample. (b) Lysates of the same muscle tissues used in FIG. 2b were tested by western blotting using

CnA-β1 specific antibody. (c) Immunofluorescence of 7 μm transverse sections from Quadriceps muscles of SOD1_{G93A} and SOD_{G93A}mIGF-1 at paralysis stage. CnA-β1 shows a nuclear localization. A regenerating fiber is indicated by the presence of central nucleus (red arrow). Nuclei were visualized by Hoechst dye (blue). Bar, 20 μm.

FIG. 15. Maintenance of the neuromuscular junction configuration in SOD1_{G93A} x MLC/mIGF-1 transgenic mice. (a) Immunofluorescent analysis of 7 μm transverse sections from muscles of wild type, MLC/mIgf-1, SOD1_{G93A} and SOD_{G93A}mIGF-1 transgenic mice at 123 days of age. α-bungarotoxin antibody identified diffusion of acetylcholine receptor (AChR) expression in SOD1_{G93A} muscle (yellow arrow); whereas AChR showed a transitory polyinnervation, as indicated by the presence of two clusters in a single fiber (red arrows). Bar, 20 μm. (b) Northern blot of total RNA samples (15 μg) from quadriceps of wild type (lane 1), MLC/mIGF-1 (lane 2), SOD1_{G93A} (lane 3), and SOD_{G93A}mIGF-1 (lanes 4, 5) transgenic muscles at 123 (lanes 1-4) and 150 (lane 5) days of age, hybridized with AChR 32P-labeled probe. Ethidium bromide staining was used to verify equal loading of the RNA sample. (c) Western blot analysis for agrin from quadriceps of wild type (lane 1), MLC/mIGF-1 (lane 2) SOD1_{G93A} (lane 3) and SOD_{G93A}mIGF-1 (lane 4) transgenic muscles. SOD1_{G93A} and SOD_{G93A}mIGF-1 mice were analyzed at comparable end-stage disease.

FIG. 16. Transgenic mIGF-1 expression protects motor neuron from degeneration. (a) Quantification of surviving motorneurons in the ventral spinal cord of wild type (Wt) SOD1_{G93A} (S) and SOD_{G93A}mIGF-1 (S/I) mice at different age. * p<0.01; ** p<0.001. (b) Immunofluorescence analysis identified GFAP positive astrocytes in ventral horn of SOD1_{G93A} and SOD_{G93A}mIGF-1 transgenic mice at different ages: A, B 28 days of age; C, D 123 days of age. The intensity of the GFAP signal revealed progressive astrocytosis. Bar, 20 μm. Insert in D shows Western blot analysis for GFAP in the spinal cord of SOD1_{G93A} (lanes 1, 3) and SOD_{G93A}mIGF-1 (lanes 2, 4) mice at 28 (lanes 1, 2) and 123 (lanes 3, 4) days of age. (c) RT-PCR analysis of TNF-α and β-actin of wild type (lane 1), MLC/mIGF-1 (lane 2), SOD1_{G93A} (lane 3) and SOD_{G93A}mIGF-1 (lane 4) transgenic mice at 123 days of age. Lane 5 shows a negative control consisting of RT-PCR mix without cDNA template. Lane 6 identifies the RNA positive control for TNF-α obtained from spleen.

FIG. 17. A Schematic representation of the IGF-1 gene and the various signal/E peptide isoforms.

FIG. 18. The effect of IGF-1 signal peptides on myoblast differentiation.

FIG. 19. The effect of the IGF-1 E peptides on myoblast growth.

FIG. 20. Schematic representation of the various IGF-1 isoforms tested in vivo and their phenotypic effect.

FIG. 21: Enhanced cardiac regeneration and functions in transgenic mice after myocardial infarction. a) Extent of fibrotic invasion 2 months after LCA. Wild-type (WT) and transgenic (TG) hearts were perfused with 4% paraformaldehyde (PFA) after avertin injection. Hearts in PFA were photograph with a Leica MZ12 stereo microscope. Arrows indicate fibrotic tissue. LA, left atrium; LV, left ventricle; RA, right atrium; RV, right ventricle. Right: trichrome staining of cardiac tissues. b) Functional recovery of mIGF-1 transgenic mice. Eight WT and TG mice were anaesthetized with avertin post-myocardial infarction (1 month and 2 months) and cardiac parameters were analysed with a high-resolution ultrasound system (VisualSonics Inc.). Mean percentage values of fractional shortening (FS, upper panel) and ejection fraction (EF, lower panel) are representative of three readings on each animal and of average among groups. * Shows significant values (p<0.05) between uninjured and injured hearts in WT (yellow square) and TG (red square) animals. § Shows significant values between WT and TG injured hearts. c) Representative echocardiographic recordings of heart function with and without injury in WT (upper panels) and TG hearts (lower panels). The heart function of WT mice 2 months after MI was dramatically impaired, confounding the reading and the recording in all animals tested.

FIG. 22: Enhanced cardiac regeneration in mIGF-1 transgenic mice after CTX injection. Functional recovery of mIGF-1 transgenic mice. Eight WT and TG mice were anaesthetized with avertin post-myocardial infarction (1 month) and cardiac parameters were analysed with high-resolution ultrasound. Left panels: WT and TG heart parameters. Right panels: mean percentages of ejection fraction (EF) and fractional shortening (FS), and mean thickness (millimetres) of the posterior wall are representative of three readings on each animal and of average among each group. LVIDs, left ventricle internal dimension in systole; LVIDd, left ventricle internal dimension in diastole; LV PWs, left ventricle posterior wall in systole; LVPWd, left ventricle posterior wall in diastole. Asterisk indicates significant values decreasing in WT compared to TG hearts (p-value<0.05).

FIG. 23: Late cell proliferation in regenerating mIGF-1 transgenic hearts. a) BrdU was provided ad libitum at 0.1% for up to 1 month after CTX infarction of WT and TG hearts or injected at 100 μg/g each day for 48 hours or 1 week. Paraffin sections of 10 μm were stained with a biotinylated mouse monoclonal antibody to visualize nuclei with BrdU incorporation. 10 sections (10 μm) bordering the injured site were analysed. b) Statistical analysis of BrdU positive cells counted at different time points after CTX injection. 10 sections for each experiment were analysed and the percentage of positive nuclei was calculated based on the amount of total nuclei present in the frame observed. No relative increases in BrdU incorporation were seen at 24 hours or 1 week post-CTX injection in either WT or TG hearts. Asterisk indicates significant relative values in the BrdU positive hearts at 1 month (p-value<0.05). Values are the average of three independent experiments.

FIG. 24: Characteristics of BrdU positive cells in regenerating mIGF-1 transgenic hearts. BrdU was provided ad libitum for up to 1 month at 0.1% after CTX injection of WT or TG hearts. a, b, c) BrdU positive cells were identified in paraffin sections of TG cardiac muscle (10 μm) stained with anti-biotinylated-BrdU antibody and photographed at 100× magnification. Arrows indicate BrdU positive cardiomyocytes (left and middle panels) and cells lining blood vessel (right panel). d) Cardiac myocytes and e) non-muscle cells were isolated from WT and TG hearts 1M after CTX injection. Dissociated cell cultures were analysed for BrdU and haematoxylin to visualise proliferating nuclei. The experiment was performed on three hearts each from WT and TG animals. f) Confocal microscopic analysis of BrdU positive cells in TG heart tissue at 100× magnification. Cardiomyocytes were visualised by an anti-myosin antibody. White arrows indicate BrdU positive cardiac myocytes; non-cardiomyocyte cells are indicated by red arrows.

FIG. 25: FIG. 1. mIGF-1 enhances the activation of satellite cells. Immunofluorescent analysis of 7 um transverse sections from wild type and MLC/mIGF-1 injured muscles. Desmin antibody identified a pronounced activation of satellite cells in MLC/mIGF-1 muscle.

FIG. 26: mIGF-1 accelerates muscle regeneration. Scheme of the different phases characterising muscle regeneration in wild type and MLC/mIGF-1 transgenic mice.

FIG. 27: mIGF-1 expression negatively modulates inflammatory response during muscle regeneration. FACS analysis of molecular markers of inflammatory cells (CD11b, Gr1, CD45) in wild type and MLC/mIGF-1 transgenic injured muscle.

FIG. 28: Muscle architectures is rapidly restored in mIGF-1 injured muscle. Histological analysis of wild type and MLC/mIGF-1 transgenic muscle after 5 and 15 days post-injury.

FIG. 29: mIGF-1 improves stem cell-mediated muscle regeneration in dystrophic muscle. Histological analysis of mdx and mdx/mIGF-1 dystrophic muscle after stem cell transplantation. Brown fibers revealed that the transplanted MLC/hAP bone marrow stem cells massively participate to muscle regeneration and repair in mdx/mIGF-1 dystrophic mice.

### EXAMPLES

### Example 1

### Transgenic Overexpression of IGF-1 Inhibits Ubiquitin-Mediated Muscle Atrophy

The present study was undertaken to investigate whether activation of the ubiquitin-proteasome pathway contributes to muscle atrophy in the syndrome of chronic heart failure. Since progressive muscle atrophy in advanced stages of chronic heart failure correlates with low serum levels and reduced local expression of IGF-1 (48,49), it is hypothesized that expression of an MLC/mIGF-1 transgene encoding a locally acting isoform of IGF-1 normally induced in response to muscle damage (50), could prevent the development of heart failure-associated muscle atrophy and concomitant activation of the proteasome.

Here it is shown that muscle activation of the ubiquitin-proteasome pathway in the setting of chronic left ventricular dysfunction is accompanied by selective induction of the muscle-specific ubiquitin ligase atrogin-1/MAFbx (for Muscle Atrophy F-box). Activation of Foxo transcription factors occurs in the skeletal muscle in chronic left ventricular dysfunction. Transgenic supplementation of the mIGF-1 isoform prevents muscle atrophy and activation of the proteasome. Further, overexpression of mIGF-1 specifically inhibited activation of Foxo4, the most abundant of these factors in skeletal muscle, and blocked expression of atrogin-1/MAFbx. These studies establish a role for ubiquitin-mediated proteolytic degradation in muscle atrophy accompanying chronic left ventricular dysfunction and highlight the potential therapeutic value of supplementing the local mIGF-1 isoform in the treatment of progressive muscle wasting.

### Methods

Animal Model Myocardial infarction was induced under anaesthesia in 8-12 weeks old male FVB mice (wildtype, WT) and in an FVB transgenic mouse line (MLC/mIgf-1) with skeletal muscle-restricted expression of the Exon1-Ea isoform of the rat IGF-1 gene (50). Progressive cardiac dysfunction was induced by ligation of the left coronary artery while sham-operated animals underwent the same procedure without ligation of the coronary artery. All mice underwent echocardiography after 2 and 12 weeks and were sacrificed after 12 weeks by injection of pentobarbital. The investigation conformed to the Guide for the Care and Use of Laboratory Animals published by the US National Institute of Health (NIH Publication No. 85-23, revised 1996) and was approved by the Harvard Medical School Standing Committee on Animals.

Histology Organs were removed, fixed in 4% paraformaldehyde and embedded in paraffin for further histological analysis. Tissue samples were cut in 5 μm thick sections and stained using hematoxylin and eosin. Morphological analysis of muscle fiber cross-sectional area was performed on tissue scans using ImagePro software (ImagePro Plus 4.5, Media Cybernetics).

Cell culture SOL8 cells (American Tissue Culture Collection) were cultured in DMEM containing 20% fetal calf serum, 100 U/ml penicillin and 100 μg/ml streptomycin. Cells were incubated with tumor necrosis factor-α (TNF [symbol] 20 ng/ml), interleukin-1β (IL-1β, 50 ng/ml), insulin-like growth factor-1 (IGF-1, 10 ng/ml) and dexamethasone (Dexa, 10 ng/ml).

Real-Time PCR Total RNA (100 ng) was assessed by real-time PCR (LightCycler, Roche) using primers for atrogin-1/MAFbx (sense: 5′-GAC TGG ACT TCT CGA CTG CC-3′ and antisense: 5′-TCA GCC TCT GCA TGA TGT TC-3′) and β-tubulin (sense: 5′-CTG GGC TAA AGG CCA C-3′ and antisense: 5′-AGA CAC TTT GGG CGA G-3′). The expression was normalized to expression levels of β-tubulin.

Western Analysis Protein levels were analyzed by Western analysis using specific monoclonal antibodies for the detection of myosin heavy chain (MF20, Developmental Studies Hybridoma Bank) or ubiquitin (P4D1, Santa Cruz Biotechnology). Polyclonal antibodies against phospho-Akt, total Akt, phospho-Foxo 1, 3 and 4 and total Foxo were from Cell Signaling. After incubation with HRP-conjugated secondary antibody, specific bands were visualized by enzymatic chemiluminescence (Perkin Elmer).

Immunoprecipitation 30 μl of protein A sepharose beads were incubated with 1 μg anti-ubiquitin antibody. 300 μg of total protein from the soluble fraction of the muscle lysates were incubated with antibody-bead complexes for two hours rotating at 4° C. Beads were centrifuged and washed three times with 0.5 ml lysis buffer and one time with 0.5 ml ice-cold PBS. The beads were resuspended in SDS sample buffer, incubated at 95° C. for five minutes, centrifuged, and the supernatant electrophoresed through a SDS-PAGE system. After transfer to a PVDF membrane, further immunoblotting was performed.

Proteasome Activity Protein lysates were incubated at 37° C. with proteasome assay buffer containing 10 μmol/ml SLLVT-AMC (Calbiochem) as substrate for the chymotrypsin-like activity of the proteasome. Fluorescence of free 7-AMC as a measure of proteasome activity was assessed in intervals over 60 min on a temperature-controlled fluorescence reader (Perkin Elmer).

Statistical Analysis All experiments were performed at least three times and data are expressed as mean±SEM. Data were analyzed by Student's t-test. One-way ANOVA with post-hoc analysis was used for analysis of data sets of more than two groups. A p-value of less than 0.05 was considered statistically significant.

### Results

Assessment of Animals with Chronic Left Ventricular Dysfunction and Controls Thirty-nine WT and 34 MLC/mIGF-1 transgenic mice were included in the study (Table 1). The animals were randomly assigned to a group undergoing ligation of the left coronary artery or sham surgery. Perioperative mortality and total mortality over the study period was comparable between WT and MLC/mIGF-1 (p=NS). Echocardiographic assessment demonstrated the expected development of progressive left ventricular enlargement and systolic dysfunction following left coronary artery ligation. Baseline differences in left ventricular dimensions between WT and MLC/mIGF-1 mice persisted throughout the study with comparable increases in wall thickness of the non-infarcted ventricular wall and ventricular volume (Table 2).

TABLE 1
Morphometric Assessment

| | | WT | | MLC/mIgf-1 | |
|---|---|---|---|---|---|
| Weight (g) | | Sham | MI | Sham | MI |
| Total Body | (8-10 wks) | 25.9 ± 2.0 | 26.1 ± 2.2 | 34.5 ± 5.9‡§ | 33.3 ± 3.8‡§ |
| | (20-22 wks) | 29.6 ± 2.8 | 32.9 ± 2.5 | 38.1 ± 8.9* | 40.1 ± 7.8† |

| | | | | |
|---|---|---|---|---|
| Heart | 0.16 ± 0.02 | 0.21 ± 0.05 | 0.18 ± 0.04 | 0.25 ± 0.1 |
| Heart/Total (×10⁻³) | 5.42 ± 0.66 | 6.46 ± 1.65* | 4.82 ± 1.22 | 6.42 ± 3.65 |
| EDL (×10⁻³) | 63 ± 9 | 54 ± 11* | 69 ± 17 | 68 ± 12† |
| SOL (×10⁻³) | 154 ± 15 | 138 ± 13* | 178 ± 32* | 172 ± 23§ |
| QUA (×10⁻³) | 186 ± 13 | 171 ± 17* | 214 ± 31* | 208 ± 29§ |

(Data are presented as mean ± SD; WT—wildtype; MI—myocardial infarction; EDL—M. extensor digitorum longus; SOL—M. soleus; QUA—M. quadriceps;
*p < 0.05 vs. WT sham;
†p < 0.05 vs. WT MI;
‡p < 0.001 vs. WT sham;
§p < 0.001 vs. WT MI)

TABLE 2
Echocardiographic Assessment of Left Ventricular Function

| | WT | | MLC/mIGF-1 | |
|---|---|---|---|---|
| | Sham | MI | Sham | MI |
| AWT (mm) | 1.25 ± 0.11 | 1.28 ± 0.08 | 1.37 ± 0.13* | 1.39 ± 0.13 |
| PWT (mm) | 1.13 ± 0.14 | 1.20 ± 0.14 | 1.25 ± 0.15 | 1.27 ± 0.14 |
| LVEDD (mm) | 2.70 ± 0.16 | 3.19 ± 0.59* | 2.87 ± 0.30 | 3.19 ± 0.46 |
| LVESD (mm) | 1.08 ± 0.26 | 1.66 ± 0.50 | 1.21 ± 0.16 | 1.61 ± 0.37† |
| LV Volume (cm³) | 0.03 ± 0.01 | 0.047 ± 0.03‡ | 0.032 ± 0.01 | 0.049 ± 0.02† |
| FS (%) | 57.8 ± 2.5 | 48.5 ± 7.5‡ | 57.5 ± 5.6 | 48.6 ± 5.3§ |

(Data are presented as mean ± SD; WT—wildtype; MI—myocardial infarction; AWT—anterior wall thickness; PWT—posterior wall thickness; LVEDD—left ventricular end-diastolic diameter; LVESD—left ventricular end-systolic diameter; FS—fractional shortening;
*p < 0.05 vs. WT sham;
†p < 0.05 vs. mIgf-1^{+/+}sham;
‡p < 0.001 vs. WT sham;
§p < 0.01 vs. mIgf-1^{+/+}sham)

### Skeletal Muscle Atrophy and Activation of the Ubiquitin-Proteasome-Pathway in Chronic Left Ventricular Dysfunction

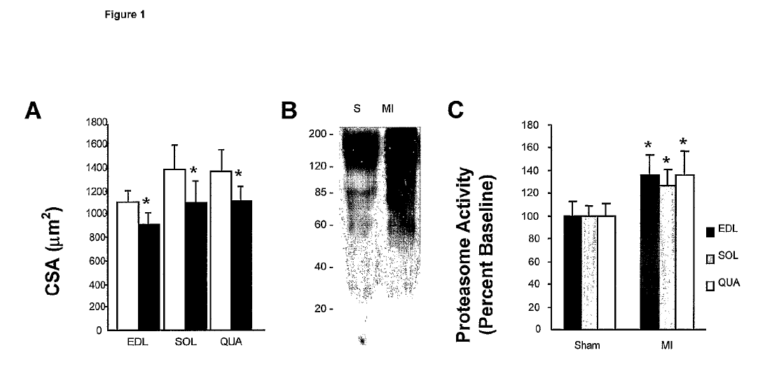

Assessment of skeletal muscle revealed a reduction in muscle fiber cross-sectional area in WT animals with CLVD (FIG. 1A). This reduction was paralleled by a decrease in isolated muscle weight (Table 1). Next the skeletal muscle activation of the proteolytic ubiquitin-proteasome pathway in WT and MLC/mIGF-1 transgenic animals was assessed. Immunoprecipitation of total ubiquitinated proteins followed by anti-ubiquitin immunoblotting revealed an increase in ubiquitinated substrates in soluble fractions of atrophying muscles (FIG. 1B). The activity of the 20S proteasome was measured by assessment of the chymotrypsin-like proteolytic activity. WT animals with CLVD and muscle atrophy displayed an increase in 20S proteasome activity in skeletal muscle by 40% (p<0.05 vs. controls) (FIG. 1C).

Transgenic Overexpression of mIGF-1 Prevents Activation of the Ubiquitin-Proteasome-Pathway and Muscle Atrophy in Chronic Left Ventricular Dysfunction

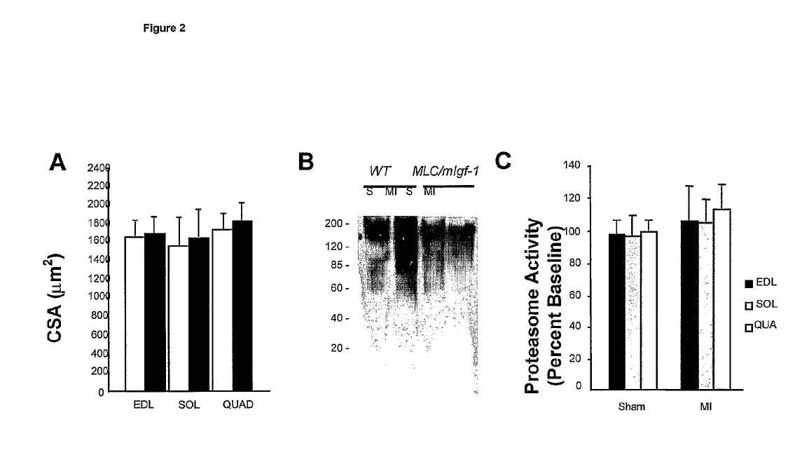

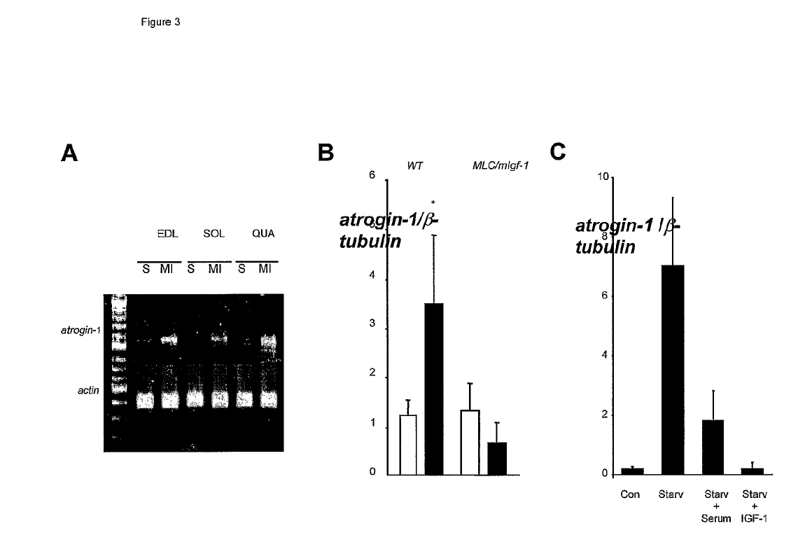

In MLC/mIGF-1 mice, muscle fiber cross-sectional area was increased at baseline due to the anabolic function of locally overexpressed mIGF-1 (51). In contrast to WT animals, MLC/mIGF-1 mice with CLVD suffered no reduction of muscle fiber cross-sectional area (FIG. 2A). Further, transgenic overexpression of mIGF-1 prevented the increase in total muscle protein ubiquitination following myocardial infarction (FIG. 2B). Finally, the increase in proteasome activity was prevented in skeletal muscle of MLC/mIGF-1 transgenic mice with CLVD (FIG. 2C).

Expression of the Muscle-specific Ubiquitin Ligase atrogin-1/MAFbx in Skeletal Muscle of Animals with Chronic Left Ventricular Dysfunction

E3-ligases are ubiquitin-protein conjugating enzymes with a crucial role in the molecular cascade of protein ubiquitination, which marks them for rapid proteolytic degradation (Bodine et al. 2001; Gomes et al. 2001). The expression of atrogin-1/MAFbx in skeletal muscle of mice with CLVD was determined and showed a robust expression of atrogin-1/MAFbx in several muscles following myocardial infarction (FIG. 3A). Transcripts of atrogin-1/MAFbx were strongly induced in atrophying muscles of animals with CLVD (3.48±1.38 vs. 1.21±0.32 arbitrary units; p<0.01 vs. WT sham). In MLC/mIGF-1 transgenic muscle, expression of atrogin-1/MAFbx remained at basal levels following the development of CLVD (FIG. 3B).

The regulation of atrogin-1/MAFbx in the setting of muscle atrophy was confirmed in SOL8 cells starved for 72 h. Intriguingly, this effect was reduced by stimulation with 20% serum for 3 h and completely normalized by stimulation with IGF-1 for 3 h (FIG. 3C).

### Polyubiquitination of Myosin Heavy Chain in Muscle Atrophy

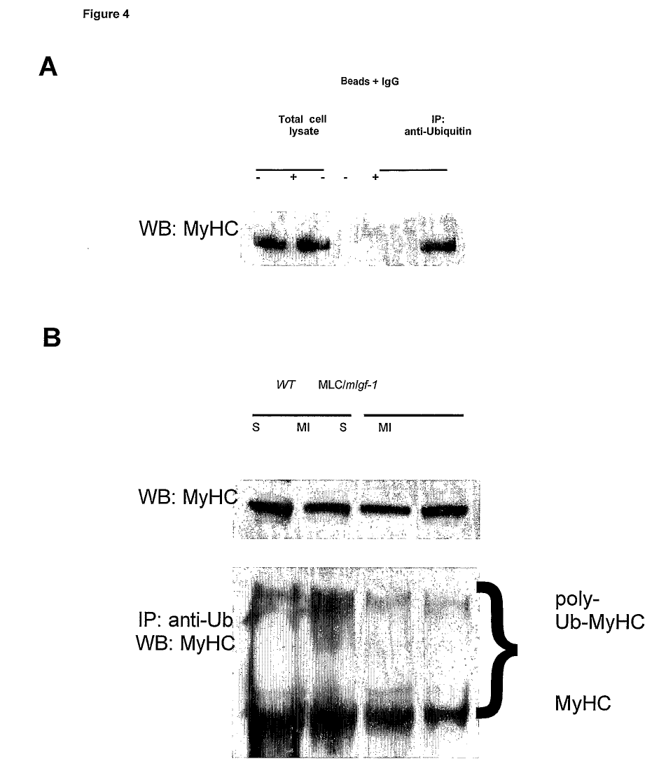

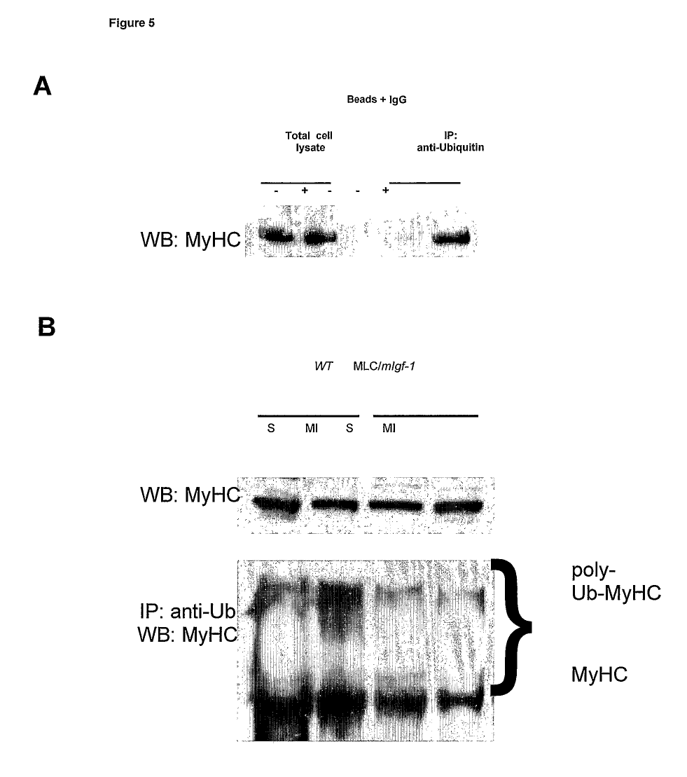

To assess whether increased ubiquitination of myosin heavy chain (MyHC), a known target of ubiquitination, (Solomon and Goldberg 1996; Acharyya et al. 2004) could be induced in vitro, SOL8 myogenic cell cultures were incubated with different inducers of ubiquitination. Dexamethasone induced total protein ubiquitination after 24 h and analysis of total ubiquitinated proteins followed by immunoblotting revealed a strong increase in MyHC ubiquitination (FIG. 4A) which was also seen after serum withdrawal and TNFα and IL-1β incubation (data not shown).

To determine whether MyHC was a also target of ubiquitin-mediated proteolytic degradation in vivo, similar analyses were performed on atrophied skeletal muscles of mice with CLVD and found an increased fraction of ubiquitinated MyHC in WT animals with chronic CLVD (FIG. 4B). In contrast, expression of the MLC/mIGF-1 transgene blocked the increase in MyHC ubiquitination in skeletal muscle of animals with chronic CLVD.

Activation of Foxo Transcription Factors in Muscle Atrophy is Inhibited by mIgf-1

Reduced activity of the PI3 Kinase/Akt pathway leading to enhanced activation of its downstream target, Foxo transcription factors, and expression of atrogin-1/MAFbx has been associated with muscle atrophy (52). Reduced Akt activity in muscle of WT mice with CLVD was prevented by mIGF-1 transgene expression (FIG. 5A). Activation of Foxo transcription factors indicated by reduced phosphorylation in muscle of WT mice with CLVD was also abrogated by mIGF-1 transgene expression, which specifically enhanced Foxo4 phosphorylation in muscle from mice with CLVD (FIG. 5B).

### Discussion

In this study, an animal model of chronic left ventricular dysfunction (CLVD) was employed to investigate the ubiquitin-proteasome pathway in skeletal muscle atrophy and to explore potential therapeutic avenues for its prevention. An activation of the ubiquitin-proteasome pathway in atrophying skeletal muscle accompanied by Foxo activation and expression of the atrophy related ubiquitin-protein ligase atrogin-1/MAFbx is demonstrated. These changes are prevented by transgenic overexpression of mIgf-1. Consideration of IGF-1 isoforms is critical in the interpretation of current studies on the effects of supplementary growth factors. Exogenously administered IGF-1 induces muscle hypertrophy through autocrine and paracrine mechanisms (53) and muscle-specific overexpression of a circulating IGF-1 isoform results in profound muscle growth mediated through increased protein synthesis and DNA accretion (54). Overexpression of the mIGF-1 isoform counters the decline in muscle mass in senescence (50) and in mdx mice (55). Gene transfer of mIGF-1 under the control of muscle-specific regulatory elements prevents age-related loss of skeletal muscle mass and function even when administered at senescence (20). Additionally, mIGF-1 may act as a potent regenerative agent, as increased stem cell recruitment to sites of muscle injury was observed in mice expressing the MLC/mIGF-1 transgene. When isolated from MLC/mIGF-1 muscles, these progenitor cells exhibit accelerated myogenic differentiation and induce muscle-specific markers in co-cultured bone marrow cells (Musaro et al. 2004). Therefore, it is likely that locally produced mIGF-1 counteracts atrophy through signal transduction pathways that may be distinct from those activated by circulating IGF-1.

### Example 2

### Full Regeneration of the Mammalian Heart

### Methods

### Generation of α-MyHC/mIGF-1 Transgenic Mice

We generated transgenic mice (FVB) with a rat mIGF-1 cDNA driven by the mouse α-MyHC promoter (56). Transgenic mice were generated by standard methods and selected by PCR using tail digests. Transgenic animals were maintained as heterozygotes. The animals were housed in a temperature-controlled (22° C.) room with a 12:12 hour light-dark cycle. All the analyses were performed on male mice.

### RNA Preparation and Northern Blot Analysis

Total RNA from wild type (WT) and mIGF-1 transgenic (TG) hearts was obtained by RNATRIZOL extraction (Gibco-BRL). RNA (10 μg) was analyzed on 1.3% agarose gels and hybridized as described (57).

### Histological Analysis

Mice at different ages were anesthetized before cervical dislocation, and hearts were perfused with 4% paraformaldehyde (PFA) as previously described (58), then excised and embedded in paraffin. Paraffin sections (10 μm) were stained with haematoxylin and eosin and analyzed morphologically. Connective tissue was visualized by using Masson's Trichrome stain as described by Manufacture (Sigma). Cell size was analyzed by measuring the size of single nuclei cells in a 40× magnification. 10 sections (10 μm) from WT and TG hearts were used for cell measurement. Cells were measured in the left ventricle. Statistical analysis was performed as described below.

### Cardiac Injury

3-4 months-old WT and TG mice were anesthetized by Avertin injection (0.1 ml/10 g of a 2.5% solution). The tongue was retracted and a tracheal cannula (1.3×1 mm, OD×ID, Harvard Apparatus) was inserted into the trachea. The cannula was attached to the mouse ventilator (Model 687, Harvard Apparatus) via the Y-shaped connector. Ventilation was performed with a tidal volume of 200 μl and a respiratory rate of 120/min. The chest cavity was opened in the left fourth intercostal space. The heart was exposed and 25 μl of CTX 10 μM (Latoxan) were injected in the heart wall of the left ventricle or the left descending artery was ligated. The chest cavity, muscle, and skin were then closed by a 6-0 silk suture (Ethicon).

LCA was performed on avertin anesthetized mice as described above. Ventilation was performed with a tidal volume of 300 μl and a respiratory rate of 120/min. The chest cavity was opened in the left fourth intercostal space and the left coronary artery (LCA) was ligated with a 8.0 no absorbable suture (ethicon) below the left atrium to produce a 40% infarct size. The chest cavity, muscle, and skin were then closed by a 6-0 silk suture (Ethicon). Mice were kept under ventilation until they were completely awake from anesthetic.

### Echocardiography

Eight 13 and 23 week-old males from WT and TG lines were weighed and lightly anaesthetized with pentobarbital (30 mg/kg i.p.) to allow analysis of cardiac anatomy and function on a Sonos 5500 (Hewlett Packard) with a 15 MHz linear transducer (15L6) (Philips Ultrasound, USA). The images were stored in a digital format on a magnetic optical disk for review and analysis. The left hemithorax was shaved and an ultrasound transmission gel was applied to the precordium. The heart was first imaged in the two-dimensional mode (2D) in the parasternal long-axis view to obtain the aortic root dimensions. The aortic flow velocity and the heart rate (HR) were measured with pulsed-wave Doppler on the same section. The sample volume cursor was placed in the aortic root and the transducer angled slightly, which allowed aortic flow parallel to the interrogation beam so that maximum aortic flow velocity was obtained easily. The cardiac output (CO) was calculated from the following equation: CO=0.785×D2×VTI×HR where D is the internal diameter of the aortic root and VTI is the velocity-time integral of the Doppler aortic spectrum. Then the pulsed Doppler window was placed between the tip of the mitral valve leaflets to record the mitral inflow velocities. The maximal speed of the early (E) and late (A) mitral filling were measured as well as the mean deceleration time of the E wave (DT) and the duration of the A wave (Adur). By placing the Doppler between aortic flow and mitral valve, the isovolumetric relaxation (IVRT) time was measured. Left ventricular cross sectional internal diameters in end-diastole (LVEDD) and in end-systole (LVESD) were obtained by an M-mode analysis of a 2D-short axis view at the papillary muscle level. The ejection and shortening fractions were calculated. From this view, the diastolic septum (S) and posterior wall (PW) thicknesses were measured. The left ventricular mass (LVM) was calculated with the following formula: LVM=1.055×[(S+PW+LVEDD)3−(LVEDD)3]. All the measurements were performed on, at least three beats, according to the guidelines of the American Society of Echocardiography.

### Ultrasound Analysis of Injured Hearts

Eight 13 week-old males from WT and TG lines were analyzed by echocardiography one month after CTX injection in the left ventricle wall or after 1 month and 2 months after LCA ligation. The mice were weighed and lightly anaesthetized by Avertin injection (0.1 ml/10 g of a 2.5% solution). Cardiac anatomy and function were measured with a Vevo 660 (VisualSonics) Ultrasound, and by the use of a 630 RMV (real-time-micro-visualization) scanhead (Visualsonics). The analysis was very sensitive due to the high-resolution images that the VisualSonics Ultrasound can acquire. The left hemithorax was shaved and an ultrasound transmission gel (Parkers Laboratories Inc.) was applied to the precordium. The heart was imaged in the two-dimensional mode (2D) in the parasternal short-axis view to obtain left ventricular cross sectional internal diameters in end-diastole (LVEDD) and in end-systole (LVESD) by an M-mode analysis. The ejection and shortening fractions were calculated. From this view, the posterior wall (PW) thicknesses was also measured. Movie recordings of left ventricle motion were analysed in B-mode and in parasternal short-axis (PSA). 300 different frames covering cycles of ventricular contraction and distension (systole and diastole) were recorded.

### Immunohistochemistry and BrdU Analysis

BrdU (Sigma) was administered ad libitum at 0.1% in the drinking water or injected intraperitoneally at 100 μg/g. Hearts were perfused with 4% PFA and embedded in paraffin. Sections were stained with anti-BrdU (BD-Pharmingen) as prescribed by the manufacturer. Positive nuclei were quantified by counting all nuclei and BrdU positive nuclei in 10 sections (10 μm) of WT and TG hearts bordering and covering the CTX injured side. Statistical analysis was performed as described below. Immunofluorescence was performed on frozen sections (10 μm) of WT and TG hearts 1 month after CTX injection. BrdU was analyzed with a mouse anti-BrdU purchased from Amersham Biosciences, and cardiac muscle cells were stained with an anti-myosin antibody from Sigma (M7648). Nuclei were visualized by Hoechst dye (Sigma). Images were processed with a Leica DM RHC fluorescent microscope and a DC500 Digital Camera.

### Isolation of Cardiac Cells

Adult mouse cardiomyocytes were isolated and cultured following the instructions of www.signaling-gateway.org.

### Western Blot Analysis

Hearts from WT and TG mice were excised and excess blood was removed by washing in PBS1×. Hearts were lysed in buffer containing 20 mM Tris-HCl (pH 8.0), 150 mM NaCl, 5 mM MgCl2, 10% glycerol, 1% Triton, 0.5% NP40, supplemented with 1 mM proteases and phosphatase inhibitor cocktail. 50 μg of proteins were loaded onto SDSPAGE gel and blotted on PVDF membrane. Phospho-Akt (Pharmingen) and Phospho-S6 (Cell Signaling) were used at a concentration of 1:500 and 1:1000 respectively in 5% BSA. Mouse monoclonal p21 antibody was purchased form Santa Cruz and used at a concentration of 1:250 in 5% milk. The blots were normalized for Akt (Transduction Laboratories), S6 ribosomal protein (Cell Signaling) and actin (goat polyclonal, Santa Cruz).

### Real Time PCR and Reverse Transcriptase PCR

1 μg of RNA was used to set up the reaction of reverse transcription as prescribed by Manufacture (Promega). Real time PCR was performed using 10 μl of the Syber Green Dynamo™ Master Mix (Finnzymes, Espoo, Finland), along with 1 μl of cDNA and 0.75 mM of each primer in a total reaction volume of 20 μl. Duplicated samples were incubated at 95° for 3 min, followed by 45 cycles of amplification (95°, 10 sec; 56°, 20 sec; 72°, 30 sec). Results for each cytokine were normalized to ubiquitin ligase expression.

### Primers

IL1β, forward 5′-acatcaacaagagcttgacccaggc-3′ reverse 5′-agctcatatggtccgacagcacga-3′; IL6, forward 5′-aggataccactcccaacagacgtg-3′ reverse 5′-gtagctatggtactccagaagacc-3′; IL10 forward 5′-ccaagccttatcggaaatg-3′ reverse 5′-tggccttgtagacacc-3′; IL4 forward 5′-catcggcattttgaa-3′ reverse 5′-cgtttggcacatccatctcc-3′; GAPDH forward 5′-tgggtgtgaaccacgaa-3′ reverse 5′-acagctttccagaggg-3′; ANP forward 5′-atgggctccttctccatcaccctg-3′ reverse 5′-tcggtaccggaagctgttgcagcc-3′; BNP forward 5′-atggatctcctgaaggtgctgtcc-3′ reverse 5′-gcgttacagcccaaacgactgacg-3′; β-myosin heavy chain forward 5′-ctgagcagaagcgcaatgcagagtcgg-3′ reverse 5′-ctcctcattcaggcccttggcaccaatg-3′; α-skeletal actin 5′-atgtgcgacgaagacgagaccacc-3′ reverse 5′-gccacatacatggcaggcacgttg-3′; Glucose transporter 1 (Glut1) forward 5′-gatcccagcagcaagaaggtgacg-3′ reverse 5′-tggagaagcccataagcacagcag-3′; β-actin forward 5′-taaaacgcagctcagtaacagtccg-3′ reverse 5′-tggaatcctgtggcatccatgaaac-3′.

### Statistics

All comparisons between WT and TG mice were performed by means of paired Student's t tests. A significant difference was considered when p<0.05, setted as a double side value.

### Results

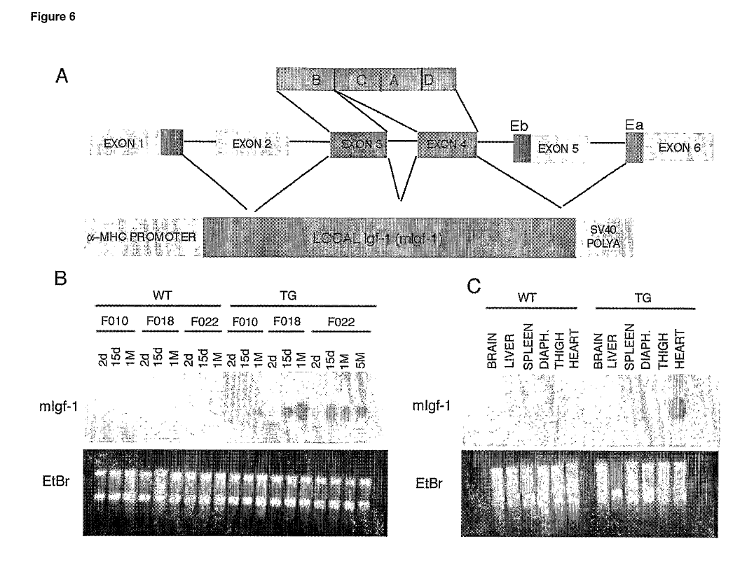

In the present study the ability of a locally acting IGF-1 isoform, mIGF-1, to regenerate the injured heart (59) was tested. Transgenic mice with a rat mIGF-1 cDNA driven by the mouse α-MHC promoter to restrict expression of mIGF-1 to the mouse myocardium and exclude possible endocrine effects on other tissues were generated (FIG. 6A). Transgenic mice developed normally with no perturbation in reproduction and breeding. Initial characterisation of three α-MHC/mIGF-1 transgenic lines with variable transgene expression levels revealed similar cardiac phenotypes. Cardiac restricted mIGF-1 transcript expression levels increased with age in all founders tested and reached a steady-level at two months (FIG. 6B). Expression of the transgene in adult mice was restricted to the heart (FIG. 6C), and endogenous levels of IGF-1 were undetectable in other tissues using the rat probe. A single transgenic line was selected for further analysis (F018).

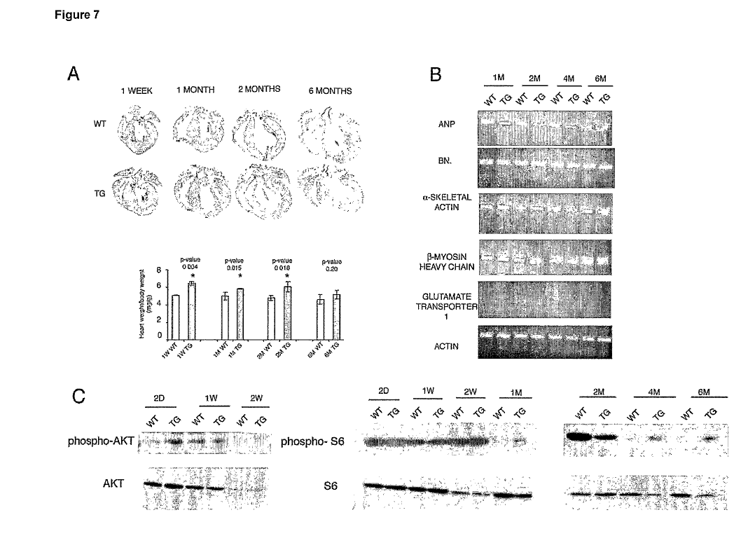

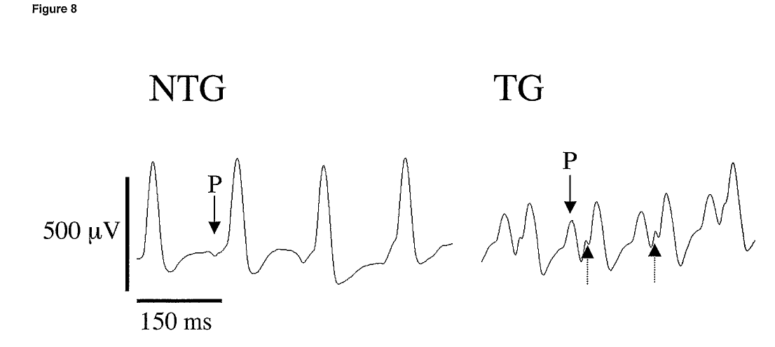

Postnatal transgenic mIGF-1 hearts displayed accelerated cardiomyocyte hypertrophy, precociously attaining wild-type adult heart size (FIG. 7A). Cardiac hypertrophy was related to higher expression levels of ANP at 1 and 2 months, without any further significative change (FIG. 7B). Other markers underlining cardiac hypertrophy, such as BNP, α-skeletal actin, β-myosin heavy chain, and glutamate transporter 1, were not affected (FIG. 7B).

Measurement of cardiac function by echocardiography and electrocardiography (FIG. 8, Table 3) showed that mIGF-1 induces a 20% concentric left ventricular hypertrophy, confirming the histological analysis (Table 3). In transgenic male mice, echocardiography identified a small but significant decrease in cardiac contractility, demonstrated by a 13% decrease in ejection fraction and fractional shortening. Nevertheless, the high level of contractility preserved the resting cardiac output despite compromised diastolic function, identified by the 21% decrease of the E/A ratio and the prolongation of the A wave duration (+14%). These two abnormalities were not linked to prolongation either of the isovolumetric relaxation time or of the mean deceleration time of the E wave. Thus, although systolic and diastolic components of the cardiac function were affected, the hearts were not dilated and cardiac output and blood pressure were maintained normal and not reduced during development (Table 3).

TABLE 3

| | 13 week-old | | 23 week-old | |
|---|---|---|---|---|
| Echocardiography | NTG (n = 8) | TG (n = 8) | NTG (n = 8) | TG (n = 8) |
| LVM (mg) | 98 ± 5 | 119 ± 6* | 101 ± 3 | 118 ± 4* |
| LWM/BW (mg/g) | 3.1 ± 0.1 | 3.8 ± 0.2* | 2.9 ± 0.1 | 3.3 ± 0.2 |
| S (mm) | 0.73 ± 0.01 | 0.82 ± 0.03* | 0.74 ± 0.02 | 0.84 ± 0.02* |
| PW (mm) | 0.59 ± 0.02 | 0.70 ± 0.02* | 0.64 ± 0.02 | 0.70 ± 0.01* |
| LVEDD (mm) | 4.17 ± 0.11 | 4.19 ± 0.08 | 4.10 ± 0.06 | 4.13 ± 0.13 |
| LVESD (mm) | 2.76 ± 0.10 | 3.03 ± 0.07* | 2.64 ± 0.06 | 2.82 ± 0.15 |
| FS (%) | 34 ± 1 | 27 ± 1* | 35 ± 1 | 32 ± 2 |
| EF (%) | 69 ± 2 | 60 ± 2* | 71 ± 2 | 66 ± 3 |
| CO (ml/min) | 26 ± 1 | 28 ± 2 | 38 ± 3 | 31 ± 3 |
| E/A | 1.61 ± 0.08 | 1.27 ± 0.11* | 1.49 ± 0.08 | 1.21 ± 0.07* |
| Adur (ms) | 35 ± 1 | 40 ± 1* | 37 ± 1 | 41 ± 2* |
| DT (ms) | 41 ± 1.7 | 43 ± 0.8 | 42 ± 2.1 | 46 ± 1.1* |
| IVRT (ms) | 21 ± 1 | 24 ± 1 | 25 ± 2 | 26 ± 2 |
| RV/LV | nd | nd | 0.25 ± 0.02 | 0.3 ± 0.01* |

NTG: non transgenic, TG: transgenic, SAP: systolic arterial pressure, HR: heart rate, PR: PR interval, QT: QT interval, LVM: left ventricular mass, BW: body weight, S: septal thickness, PW: posterior wall thickness, LVEDD: left ventricular end-diastolic diameter, LVESD: left ventricular end-systolic diameter, FS: fractional shortening, EF: ejection fraction, CO:cardiac output, E/A: maximal speed of early on late mitral filling ratio, Adur: duration of the mitral A wave, DT: mean deceleration time of the E wave, IVRT: isovolumetric relaxation time and RV/LV: right to left ventricular diameters ratio. Nd: not determined. All results are expressed as means ± sem.
*P < 0.05 (paired Student's t tests for comparisons).

In skeletal muscle cell lines, it has been reported that muscle growth and hypertrophy are mediated by activation of the AKT/mTOR pathway, leading to up-regulation of the translational machinery (60), and that IGF-1 induces skeletal myotube hypertrophy by the PI(3)K/AKT/mTOR pathway. Compelling evidences coming from lower organisms such as Drosophila Melanogaster, showed that loss or inhibition of either PI(3)K, mTOR or p70S6K resulted in decrease of cell size (61). Conversely, overexpression of the insulin receptor substrate IRS-1 or Akt or p70S6K was sufficient to cause hypertrophy of cells in which they were expressed (62, 63). This growth effect appears to be functionally conserved in mammals, as p70S6K knockout mice have reduced body size and cell growth (64).

In order to elucidate the signaling regulated by mIGF-1 overexpression in the heart, a phosphoprotein screen has been performed on wild-type and transgenic heart lysates by Kinetworks analysis (Kinexus Bioinformatic Corp.). The analysis showed that AKT pathway carrying to S6 phosphorylation is not affected (Table 4). mTOR and p70 S6 kinase phosphorylation levels were not changed (33 CPM for control wild-type vs 26 CPM for transgenic hearts) or detected respectively in transgenic mice compared to wild-type littermates (Table 4). mIGF-1 over-expression maintained a sustained S6 ribosomal protein phosphorylation during all ages analyzed, whereas the activity of the protein in wild-type hearts displayed a more modulated regulation, with a strong activation at two months and a complete decreased phosphorylation at four and six months (FIG. 7C). The sustained activation of S6 ribosomal protein observed in mIGF-1 overexpressing hearts suggested that a continuous need of ex novo protein synthesis is required to maintain the sudden growth and remodeling of the transgenic heart. However, in mammalian cells the precise pathway connecting PI(3)K to the activation of S6 and consequently of the translational machinery is a matter of some dispute (61, 65). It is interesting to notice that, independently from AKT, PDK1 has been found to directly phosphorylate p70S6K (66), indicating that AKT has a dispensable role for signaling to p70S6K. In C. elegans, PDK1 and the two PKB isoforms, Akt-1 and -2, as well as the serum-glucocorticoid kinase SGK, function in an insulin/IGF-I receptor-mediated signalling pathway to regulate metabolism, development and longevity (67, 68). Moreover, mammalian cells lacking PDK1 fail to activate downstream targets in response to IGF-121. PDK1 is considered an alternate member of the AGC kinase family and requires phosphorylation at S241 to be catalytically active (69). Strong activation of PDK1 was observed in mIGF-1 transgenic hearts (Table 3). Importantly, the mIGF-1 transgene hyperphosphorylated PDK1 at S241, the critical activation loop serine present in other AGC kinases, whereas fully processed IGF-1 strongly phosphorylates S39623, further indicating that a novel signalling cascade, independent of Akt and p70S6K, is activated downstream of the mIGF-1 isoform to increase protein synthesis and growth. Interestingly, the data showed that the phosphorylation state of AKT is not affected, besides a slight postnatal increase, and it is not regulated in parallel to activation of S6 ribosomal protein (FIG. 7C), whereas a strong activation of the downstream mediator of PI3K, PDK1, has been observed (241CPM in wild-type hearts vs 507 in transgenic hearts) (Table 4).

TABLE 4
Phosphoprotein screen of 31 phopshoproteins in wild-type and transgenic
heart tissues by Kinetworks analysis (Kinexus Bioinformatic Corp.).

| | | WT Heart | TG | | |
|---|---|---|---|---|---|
| | | Norm | Heart | WT Heart | TG |
| | | CPM | Norm CPM | % | Heart % |
| 90 kDa Ribosomal S6 Kinases (S380) | RSK1/2 Lane 7 | | | | |
| 90 kDa Ribosomal S6 Kinases (T573) | RSK1/2 Lane 5 | | | | |
| AMP-activated protein kinase alpha (T172) | AMPKa Lane 9 | | | | |
| Bone marrow X (Eph-like) kinase (Y40) | BMX (Etk) Lane 5 | | | | |
| Bruton's tyrosine kinase (Y223) | Btk Lane 15 | | | | |
| Calcium/calmodulin-dependent kinase II (T286) | CaMK2 Lane 15 | | | | |
| Cyclin-dependent kinase 1 (T161) | CDK1 Lane 19 | | | | |
| Cyclin-dependent kinase 1 (Y15) | CDK1 Lane 16 | | | | |
| elF4E binding protein (S65) (16) | 4E-BP1 (16) Lane 6 | | | | |
| elF4E binding protein (S65) (17) | 4E-BP1 (17) Lane 6 | | | | |
| elF4E binding protein (S65) (18) | 4E-BP1 (18) Lane 6 | | | | |
| Extracellular signal-regulated kinase 1 | ERK1 Lane 14 | 99 | 123 | Control | 24% |
| (T202/Y204) | | | | | |
| Extracellular signal-regulated kinase 2 | ERK2 Lane 14 | 53 | 62 | Control | 17% |
| (T185/Y187) | | | | | |
| Glycogen synthase kinase-3 alpha (S21) | GSK3a Lane 2 | | | | |
| Glycogen synthase kinase-3 beta (S9) | GSK3b Lane 2 | | | | |
| I-kappa-B kinase alpha (S180) | IKKa Lane 9 | | | | |
| I-kappa-B kinase beta (S181) | IKKb Lane 9 | 49 | 43 | Control | −12% |
| Lyn (Y507) (44) | Lyn (44) Lane 20 | 214 | 184 | Control | −14% |
| Lyn (Y507) (46) | Lyn (46) Lane 20 | 108 | 50 | Control | −54% |
| MAP kinase activated protein kinase 2 (T334) | MAPKAPK2 Lane 17 | | | | |
| MAP kinase interacting kinase 1 (T197/202) | Mnk1 Lane 4 | | | | |
| MAPK/Erk kinase 1/2(S217/221) | MEK1/2 Lane 12 | 74 | 25 | Control | −66% |
| MKK3/6(1) (S189/S207) | MKK3/6 Lane 10 | | | | |
| MKK6(2) (S207) | MKK6 Lane 10 | 69 | 85 | Control | 23% |
| p38 MAPK (T180/Y182) | p38a MAPK Lane 4 | | | | |
| p70 S6 kinase (T389) | S6Ka p70 Lane 19 | | | | |
| p70 S6 kinase (T421/T424) | S6Ka p70 Lane 17 | | | | |
| p85 S6 kinase 2 (T412) | S6K2 p85 Lane 19 | | | | |
| p85 S6 kinase 2 (T444/S447) | S6K2 p85 Lane 17 | | | | |
| Phosphoinositide-dependent protein kinase 1 | PDK1 Lane 13 | 241 | 507 | Control | 110% |
| (S241) | | | | | |
| PKC-related kinase 1 (T778) | PRK1 Lane 13 | 488 | 555 | Control | 14% |
| PKC-related kinase 2 (T816) | PRK2 Lane 13 | 54 | 93 | Control | 72% |
| Protein kinase B (T308) | PKBa (Akt1) Lane 11 | | | | |
| Protein kinase C alpha/beta (T638) | PKCa/b Lane 6 | 115 | 104 | Control | −10% |
| Protein kinase C delta (T505) | PKCd Lane 14 | 113 | 129 | Control | 14% |
| Protein kinase C zeta (T410)/lambda (T403) | PKCz/l Lane3 | | | | |
| Protein kinase D (Protein kinase mu) (S916) | PKCm/PKD Lane 3 | 60 | 17 | Control | −72% |
| Protein kinase theta (T538) | PKCt Lane 10 | | | | |
| Raf(S259) (60) | Raf1 (60) Lane 8 | 127 | 124 | Control | −2% |
| Raf(S259) (70) | Raf1 (70) Lane 8 | 114 | 98 | Control | −14% |
| Retinoblastoma Protein (S780) | RbLane 18 | 271 | 209 | Control | −23% |
| Retinoblastoma Protein (S807/S811) | Rb Lane 7 | | | | |
| The mammalian target of Rapamycin (S2448) | mTOR Lane 13 | 33 | 21 | Control | −36% |
| Type1 protein phosphatase alpha (T320) | PP1a Lane 8 | | | | |
| Zap70 (Y319)/Syk (Y352) | Zap70/Syk Lane 20 | | | | |

The trace quantity of each band is defined as CPM and is measured under its intensity profile curve. Each value is normalized by the amount of protein in each sample. Each lane corresponds to a specific protein and the Tyrosine or Serine position phosphorylated is indicated. Transgenic increase or decrease of band intensity compared to wild-type (control) is expressed in percentage.

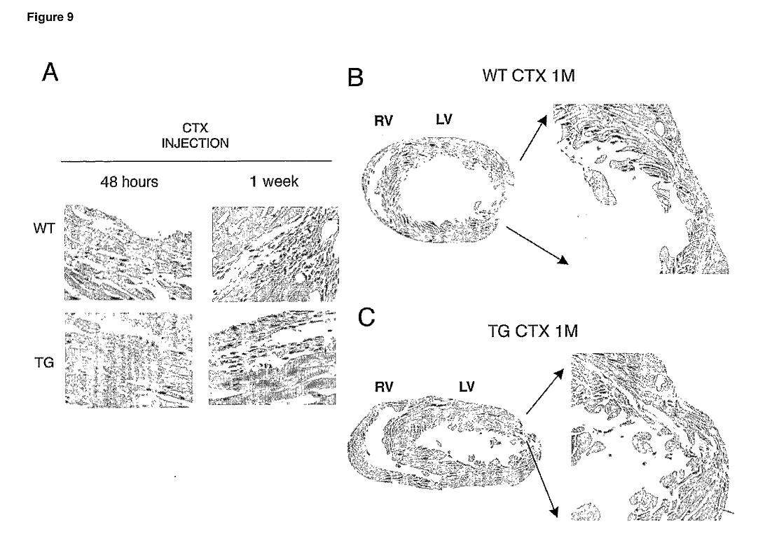

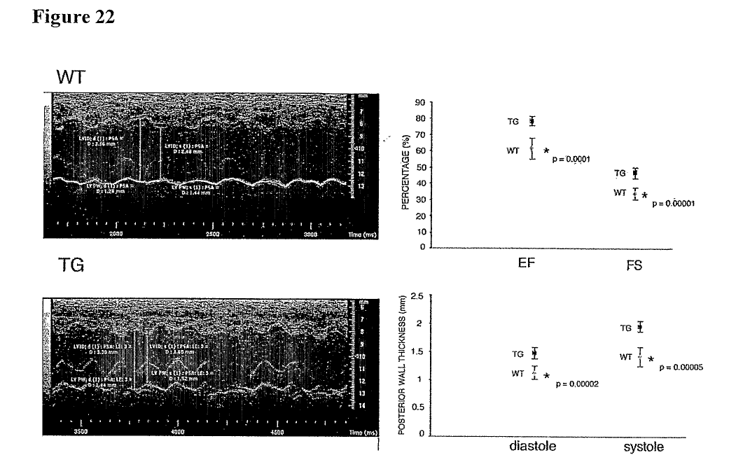

The regenerative capacity of mIGF-1 was analyzed by direct cardiotoxin (CTX) injection into the ventricles of four months old mice. The CTX model was chosen over other models of myocardial lesion such as LAD ligation because it produces a well-delineated transmural lesion, and reduces the risk of ventricular fibrillation. CTX injection in both wild-type and transgenic mIGF-1 hearts produced a reproducible and localized infarction with evident cell death and marked inflammation (FIG. 9A). In contrast to the characteristic progression of scar formation in wild-type hearts (FIG. 9B), mIGF-1 overexpression induced repair of the injured tissue after one month, without scar formation (FIG. 9C). Functional recovery after CTX-induced infarcts of wild-type and mIGF-1 transgenic hearts was analysed with high-resolution echocardiography after one month. mIGF-1 transgenic hearts showed integrity of the posterior wall and normal echocardiographic profiles compared to wild-type hearts (FIG. 22). Measurement of posterior wall thickness showed a significant decrease in the wild-type hearts in both diastolic and systolic parameters compared to transgenic hearts (diastole-WT 1.13+/−0.12 mm, diastole-TG 1.48+/−0.10 mm; systole-WT 1.42+/−0.17 mm, systole-TG 1.95+/−0.09 mm). Mean values of ejection fraction (EF) and fractional shortening (FS) were impaired in wild-type hearts when compared to transgenic hearts (EF 61%+/−7% compared to 78%+/−3%; FS 34%+/−4% compared to 47%+/−3%) (FIG. 22), indicating that mIGF-1 induced both morphological and functional regeneration.

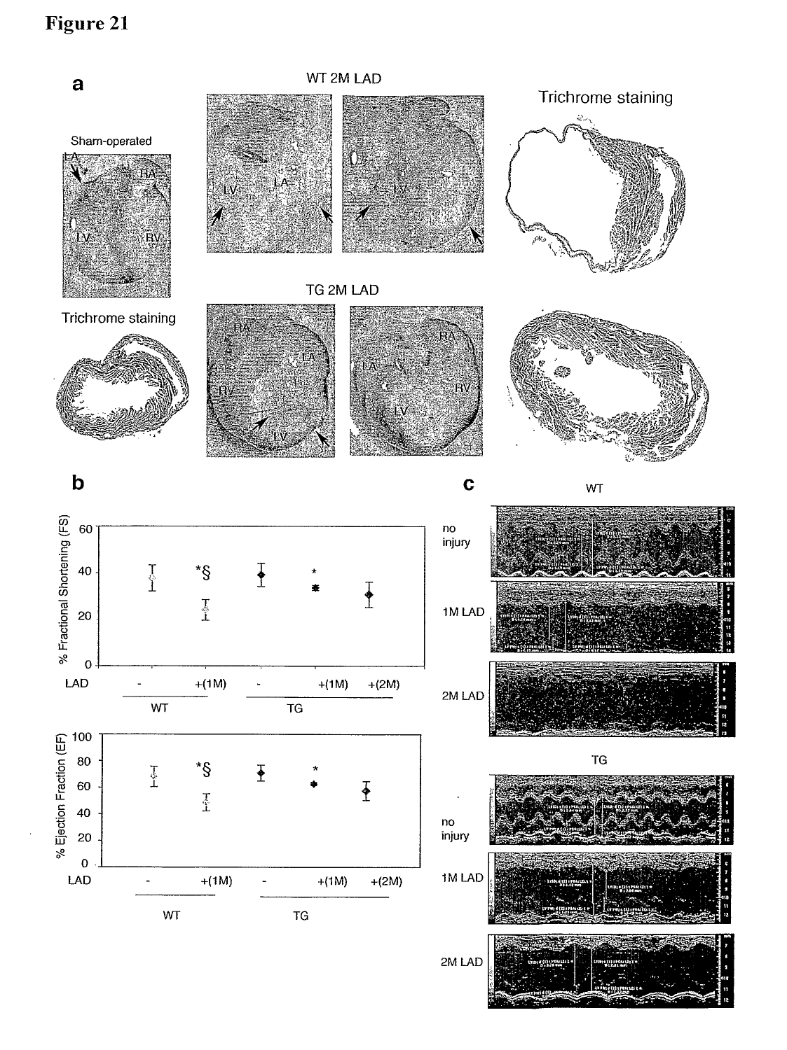

The regenerative capacity of mIGF-1 transgenic hearts was also analyzed by ligation of the left coronary artery (LCA) of four month-old mice. In wild-type mice, LCA induced infarcts characterised by progressive and extended fibrotic tissue formation (FIG. 21a, upper panel), accompanied by functional impairment after 1 month that worsened after 2 months (FIGS. 21b and c). Percentage mean values of fractional shortening (FS) and ejection fraction (EF), as measured by high-resolution echocardiography, were significantly decreased compared to sham operated mice (Table 9). In contrast, infarcted mIGF-1 transgenic hearts showed a moderate but significant decrease in the percentage of ejection fraction (EF) and fractional shortening (FS) after 1 month, with no significant changes after 2 months compared to mIGF-1 transgenic sham operated mice and wild-type ligated mice (FIG. 21b, c and Table 9). The mIGF-1-mediated blockade of the normal progressive impairment in infarcted heart function was accompanied by reduced scar formation (FIG. 21a, lower panel). Recovery of cardiac function as well as morphological restoration of infarcted mIGF-1 transgenic hearts was confirmed by normal left ventricular motion in systolic and diastolic phases compared to mIGF-1 transgenic uninjured hearts. In contrast, wild-type hearts presented chamber enlargement and a significant decrease in wall motility near the infarct.

TABLE 9
Cardiac functional parameters in wild-type (WT) and mIGF-1 transgenic (TG) mice.

| Measurement | | WT | WT 1M LCA | TG | TG 1M LCA | TG 2M LCA |
|---|---|---|---|---|---|---|
| % EF | Average | 68.1 | 48.7 | 70.8 | 62.7 | 57.47 |
| | St. Deviation | 7.7 | 6.5 | 6.0 | 1.3 | 7.1 |
| | t-test P < 0.05 | wt vs wti 0.031 | wti vs tgi 0.014 | tg vs tgi 0.015 | tgi 1M vs tgi 2M 0.37 | |
| % FS | Average | 37.7 | 24.0 | 39.1 | 33.6 | 30.68 |
| | St. Deviation | 5.6 | 4.4 | 5.0 | 1.1 | 5.45 |
| | t-test P < 0.05 | wt vs wti 0.046 | wti vs tgi 0.014 | tg vs tgi 0.039 | tgi 1M vs tgi 2M 0.49 | |

Ejection fraction (EF) and fractional shortening (FS) were measured in WT and TG mice with and without MI after 1 and 2 months. Anhestesized mice were analysed with a visualsonic ultrasound and each value is the average of three different readings on the same animal from eight male mice in each group. The heart function of WT mice 2 months after MI was dramatically impaired, confounding the reading and the recording in all animals tested (data not shown).
St. Deviation, standard deviation; wti, wild-type injured mice; tgi, transgenic injured mice. Significant values are calculated with the student t-test setting p < 0.05 as a double side value.

The early event characterizing myocardial necrosis comprises Complement activation, free radicals generation, chemokines upregulation, and cytokines cascade (70). IL8, IL6 and C5a are released in the ischemic myocardium and may have a crucial role in neutrophil recruitment (70). Intriguingly, it has been shown that cytokines inhibiting the inflammatory response, such as IL10, could have an important role in suppressing injury and blocking scar formation (71).

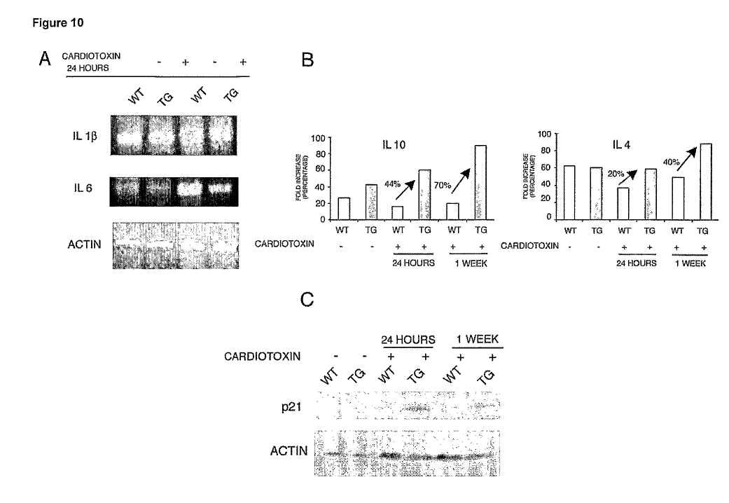

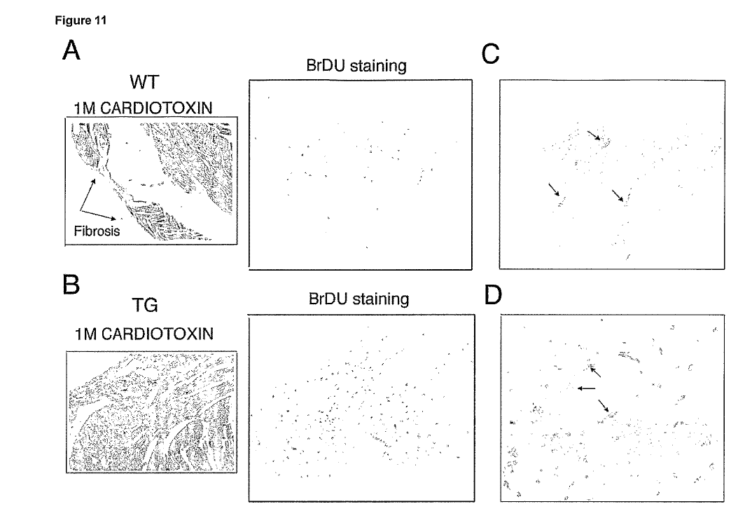

The early mechanisms leading to transgenic heart healing could involve decrease in pro-inflammatory cytokines and an increase in anti-inflammatory cytokines. Real time PCR and reverse transcriptase PCR(RT-PCR) in wild-type and transgenic hearts after 24 hours and one week from injury, showed that the pro-inflammatory IL6 was down-regulated after 24 hours from cardiotoxin injection in transgenic heart, whereas wild-type hearts showed increasing mRNA levels of IL6 (FIG. 10A). IL1β was not affected by cardiotoxin injection in wild-type and transgenic injured hearts (FIG. 10A), indicating that certain cytokines have a specific role in the heart in response to cardiotoxin injury.

The analysis of the anti-inflammatory cytokine IL10 by real time PCR showed 44% increase in transgenic hearts after 24 hour from injury, and to a greater extent at one week (77% compared to wild-type), whereas the level of the cytokine is lower in wild-type injured heart compared to uninjured tissue (FIG. 10B). IL4 was also upregulated in transgenic hearts 1 week after injury, but to a lower extent than IL10 (FIG. 10B).

Interestingly, the CDKs inhibitor p21^{WAF1/CIP1} is upregulated in response to injury in both wild-type and transgenic hearts, but overexpression of mIGF-1 lead to an extended up-regulation of p21 after injury (FIG. 10C). p21 has been implicated in many cellular responses leading to differentiation of several tissues and in the blockage of cell cycle progression (72). Recent compelling evidence showed that IGF-1 activates p21 and that p21 is important for IGF-1-mediated cell survival upon UV irradiation (73). Moreover, an intriguing study showed that spontaneous production of IL6 in rheumatoid arthritis, which is associated with high inflammation of the joints, is suppressed by p21 expression (74,75). The increased p21 expression after injury opens a novel and so far unexpected role for this cdk inhibitor in the heart.

Although regeneration has been presented as an evolutionary variable (76) illustrated by the robust proliferative capacity of the injured heart in other vertebrate such as newt and zebrafish (77,78), recent work has revealed a capacity for excellent regeneration in certain mammalian tissues, like embryonic or fetal skin (79). Compelling evidences of cardiac renewal occurring throughout life in the myocardium has been extensively proved as part of cardiac homeostasis (80,81), although the complete regenerative program in case of extended injury is precluded in mammalian heart by fibrotic tissue formation and consequently cardiac function impairment. This evidence indicates clearly that normally the regenerative program is limited in mammalian heart due to missing signaling present in the lower vertebrates such as newt and zebrafish.

Analysis suggested that the process of regenerative growth and the formation of new myocardial tissue involve modulation of the inflammatory response and changes in cytokines signaling. The myocardial tissue restoration observed in mIGF-1 overexpressing hearts could result from cardiac cell proliferation. To assess cardiac hyperplasia, cell cycle was assayed by measuring the nuclear incorporation of bromodeoxyuridine (BrdU), a marker of DNA synthesis, 1 month after cardiotoxin injection.

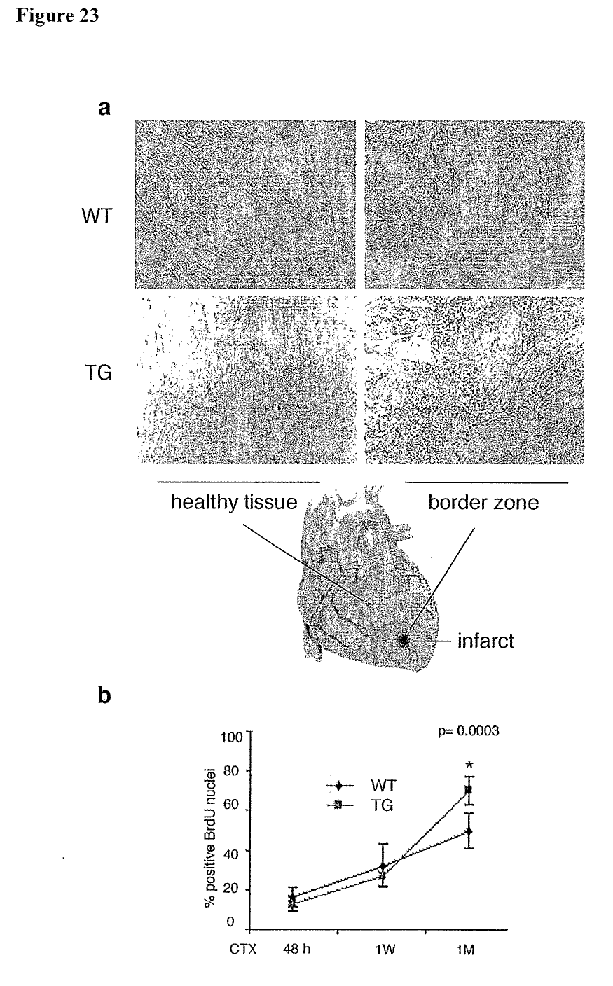

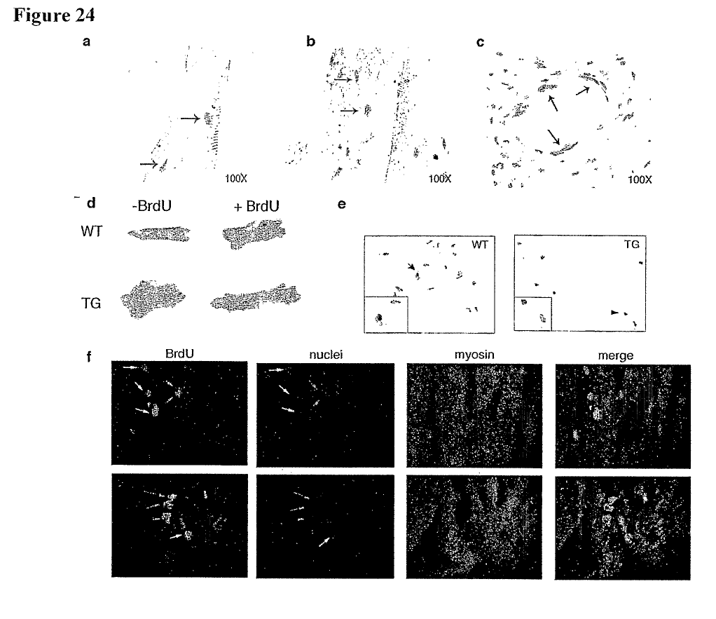

mIGF-1 induced a significant percentage of total cells to enter the cell cycle in response to cardiotoxin injection (FIG. 11B) compared to wild-type hearts (FIG. 11A). It was found that cardiac cells re-enter the cell cycle (FIG. 11C), although cells of diverse lineage were found in the myocardium and in the vessels as shown in FIG. 11D. The nature of these cells is still under investigation. No differences in BrdU incorporation were found in injured hearts 48 hours (13%+/−4% transgenic vs 16%+/−5% wild-type) and 1 week (27%+/−5% transgenic vs 32%+/−11% wild-type) after CTX injection (FIG. 23b). At 1 month after infarct induction, however, 14%+/−0.8% of total cells in the border zone of the mIGF-1 hearts were BrdU positive compared to wild-type hearts (FIG. 23a, b). Frequent incorporation of BrdU in cardiomyocyte nuclei was seen in both cardiac tissue and individual cells after injury (FIG. 24a, b, d, f), although abundant non-muscle cells of diverse morphologies were also labelled in the vessels and surrounding myocardial tissue of mIGF-1 transgenic hearts (FIG. 24c, e, f).

No differences in proliferative state were found in injured heart 24 hour and 1 week after cardiotoxin injection (data not shown), indicating that the regeneration program, following the early activated repair program, is activated as a late step in mIGF-1 overexpressing hearts.

The regenerative properties of the mIGF-1 isoform have been previously documented in skeletal muscle (82,83). In contrast to skeletal muscle, which can regenerate following injury, the mammalian heart has limited restorative capacity. Since supplementary mIGF-1 enables full myocardial regeneration following injury without altering normal heart development or long-term postnatal tissue form and function, it forms the basis of clinically feasible therapeutic strategies to bypass the normal restrictions on mammalian cardiac regeneration.

Despite shifts in signalling pathways accompanied by modest effects on morphology and hemodynamic parameters, continuous expression of mIGF-1 throughout postnatal life did not produce a significant perturbation of normal heart physiology. In contrast to previous studies with other IGF-1 transgenes (19), the hypertrophic growth of the mIGF-1 transgenic hearts did not progress to a pathological phenotype. In response to injury, the cardiac regeneration program induced by mIGF-1 followed a sequential course, involving early resolution of inflammation at the site of injury to prevent scar formation and to make way for the subsequent tissue replacement that restores form and function. Clearly mIGF-1 transgenic hearts are better prepared to contain damage by repressing pro-inflammatory molecules and increasing expression of anti-inflammatory cytokines such as IL4 and IL-10. The mIGF-1 transgene also activates p21, which is important for IGF-1-mediated cell survival upon UV irradiation (73). Prolonging the initial induction of p21 in damaged cardiac tissue enhances DNA repair and genome stability, without precluding cell replacement (84). In addition, expression of p21 suppresses production of IL6 in rheumatoid arthritis, associated with high inflammation of the joints (75). Modulating expression of these important downstream effectors of inflammation may be an important role of the intracellular signalling cascades set in motion by mIGF-1, which provide a conducive environment for cell replacement and tissue restoration.

The delayed cell proliferative response seen in regenerating mIGF-1 transgenic hearts stands in contrast to the effects of direct myocardial injection of fully processed IGF-1 protein, which rapidly induced the appearance of small new myocytes within the infarct at 1 to 2 days after coronary ligation (85). In that system the maximum benefit required a dual stimulation by IGF-1 injection together with the chemotactic effects of co-injected Hepatocyte Growth Factor, pointing either to a different mode of action employed by an expressed transgene product, or to a qualitative difference in the action of the mIGF-1 isoform itself. Elucidation of the roles played by native signal and E peptides in enhancing the regenerative response in mIGF-1 transgenic hearts, without the requirement for additional growth factors, will inform the design of clinically feasible therapeutic strategies to counteract the normal fibrotic tissue formation and consequent cardiac functional impairment in heart disease.

### Example 3

### Muscle Expression of a Local IGF-1 Isoform Protects Motor Neurons in an ALS Mouse Model

To assess the effects of supplemental IGF-1 directly on atrophic SOD1 skeletal muscle, a transgenic mouse expressing a full-length precursor of the localized IGF-1 isoform (mIgf-1) that is normally induced transiently in response to muscle damage, but does not enter the circulation (86;87) was exploited. Muscle-restricted mIGF-1 transgene (MLC/mIgf-1) exerts its effects in an autocrine or paracrine manner, circumventing the adverse side effects of systemic rIGF-1 administration. Expression of the MLC/mIGF-1 cassette, delivered either as an inherited transgene or somatically on an AAV vector, induces muscle hypertrophy and strength, and preserves regenerative capacity in senescent and dystrophic mice (20;86;88) through enhanced stem cell recruitment (89).

In the present study skeletal muscle is established as a primary target in inherited forms of ALS by showing that localised expression of the co-inherited MLC/mIGF-1 transgene exclusively in the skeletal musculature of SOD1G93A mice counteracted the symptoms of ALS, induced satellite cell activity and markers of regeneration, stabilized neuromuscular junctions and led to a reduction in astrocytosis in the SOD1G93A spinal cord. These observations offer novel approaches to the attenuation of motor neuronal degradation and underscore the importance of IGF-1 isoform selection when designing therapeutic strategies to treat ALS.

### Results and Discussion

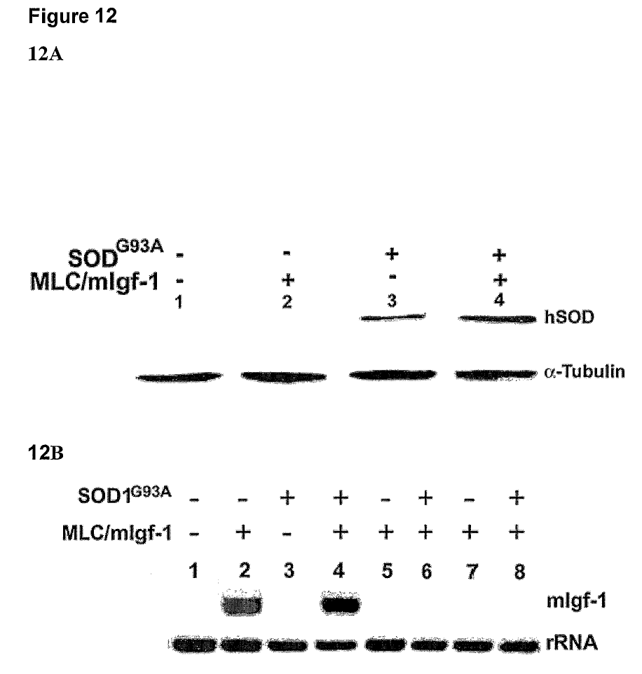

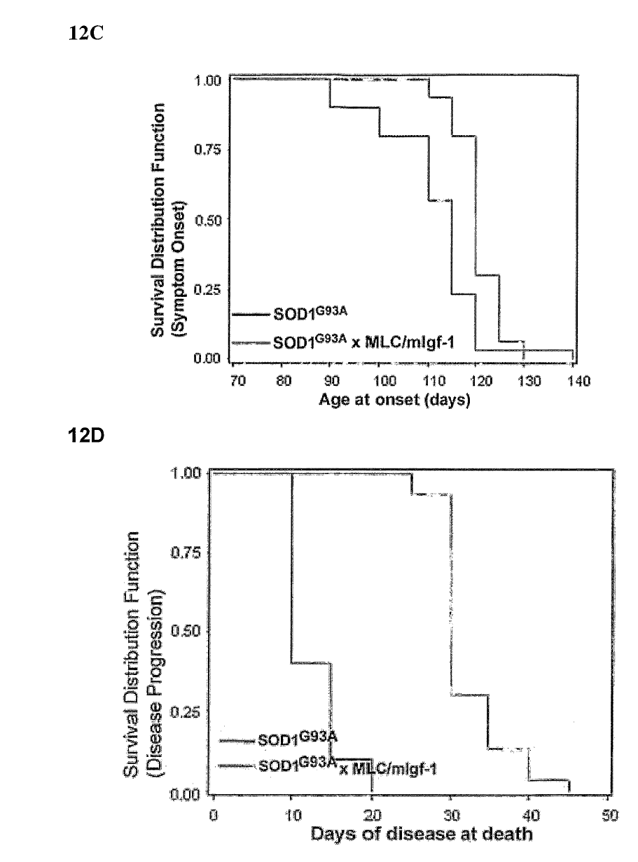

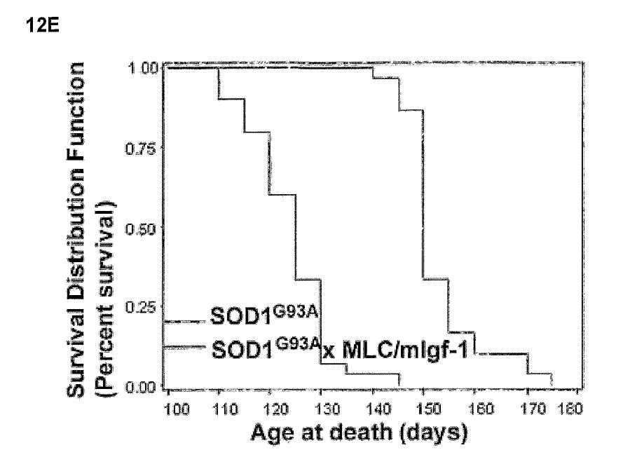

Muscle-specific mIGF-1 delays the progression of the disease and prolongs the life span of SOD1G93A mice. To evaluate the effects of mIGF-1 on the SOD1G93A neurodegenerative phenotype, double transgenic SOD1G93A and MLC/mIGF-1 transgenic mice were compared to their SOD1G93A littermates. The SODG93A and SOD1G93A x MLC/mIGF-1 (SODG93AmIgf-1) transgenic mice were selected for high copy number of the SODG93A allele and for same expression level of the human transgenic protein (FIG. 12a). Notably, mIGF-1 transgene was selectively expressed in skeletal muscle of both MLC/mIGF-1 and SODG93AmIGF-1 transgenic mice (FIG. 12b, lanes 2, 4), whereas it was not expressed in the brain and spinal cord of these mice (FIG. 12b, lane 5-8), not even in skeletal muscle of wild type and SODG93A mice (FIG. 12b, lanes 1, 3). At 111 (1.76 STDERRmean) days of age, disease onset was observed in the mutant SOD1G93A transgenic mice (n=30) (FIG. 12c). Notably, the SOD1G93A mice died within 10 days (0.62 STDERRmean) of clinical disease onset (FIG. 12d), with a maximal life-span of 145 days (FIG. 12e). By contrast, localized expression of mIGF-1 delayed the onset (FIG. 12c) and in particular the progression (FIG. 12d) of disease, increasing the survival of SODG93AmIGF-1 mice (n=30) by approximately 30 days to a maximal lifespan of 175 days (FIG. 12e). Comparative analysis revealed that the difference between SOD1G93A and SODG93AmIGF-1 were significantly relevant for onset (χ2 LR =18.67, P<0.0001), progression (χ2LR=67.07, P<0.0001), and survival ((χ2LR=63.24, P<0.0001).

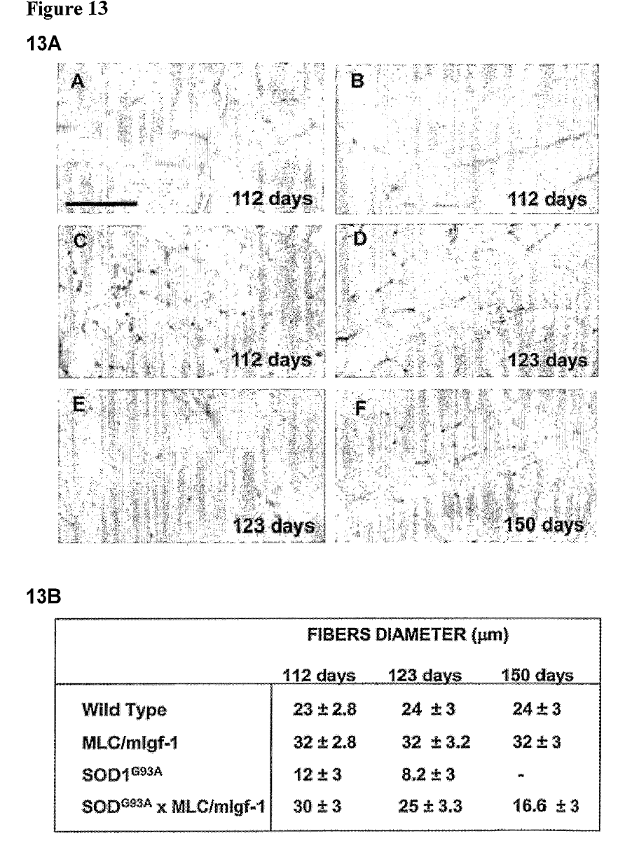

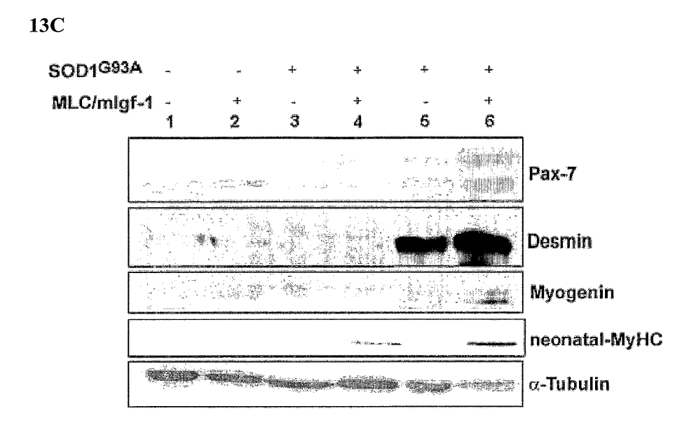

mIGF-1 expression attenuate muscle atrophy, increasing satellite cell activation in SOD1G93A mice. SOD1G93A (n=7) and SODG93AmIGF-1 (n=7) transgenic mice were analysed at different stages of disease. At 123 days, motor neuronal degeneration of SOD1G93A mice was accompanied by severe muscle atrophy (FIG. 13a, panel E), and complete muscle paralysis (hereafter this stage is indicated as paralysis stage). In contrast, at the same age, SODG93AmIGF-1 transgenic mice did not show dramatic and evident signs of muscle disease, but only difficulty in extending the limbs when suspended (hereafter this stage is indicated as symptom onset). Moreover, muscle atrophy was substantially attenuated in SODG93AmIGF-1 offspring even after onset of denervation and paralysis stage (153 days of age) (FIG. 13a, panel F; FIG. 13c). In addition, markers of satellite cell activity, such as Pax-7 and desmin, were increased to varying extents in affected SODG93A mice (FIG. 13c), whereas hallmarks of satellite cell activity and fiber maturation, including centralized nuclei (FIG. 13a yellow arrows), Pax-7 isoforms, desmin, myogenin, and neonatal MyHC expression were present exclusively in the SODG93AmIGF-1 muscles at all stages of disease, including at paralysis stage (FIG. 13c and data not shown), suggesting that satellite cells activation and maturation contribute to the maintenance of muscle phenotype induced by mIGF-1 expression (90). Motor neurons are known to regulate the properties of the myofibers they innervate by selective activation of fiber-specific-gene expression. Immunohistochemical analysis (FIG. 13d) revealed that fiber type composition was completely altered in SOD1G93A soleus muscle even prior to overt disease, with a shift in fiber type, increasing fast fibers. In contrast, the heterogeneity of muscle fibers was maintained for a more extended period in SODG93AmIGF-1 transgenic mice, which showed shift in fiber type composition only at later stage of disease (138 days) (FIG. 13d). In contrast at paralysis stage (153 days) there was not significant difference in fiber type composition between SOD1G93A and SODG93AmIGF-1 transgenic mice (data not shown). The alteration in the heterogeneity of muscle fibers of SOD1G93A mice indicate an alteration in motor neuron activity even prior to overt disease and confirm the hypothesis that the delaying in the progression and severity of ALS diseases, by mIGF-1 expression, may depend on the maintenance of muscle integrity. The functional integrity of the muscles in the SODG93AmIGF-1 transgenic mice was confirmed by a walk test (FIG. 13e) performed at different ages. At 112 days, SOD1G93A mice (n=7) showed symptom onset without evident alterations in functional parameter. The condition of SOD1G93A mice rapidly deteriorated at 115 days, as shown by the shortening of their stride (FIG. 13e). In contrast, the pathological sign of disease were delayed in SODG93AmIGF-1 transgenic mice (n=7), as shown by the capacity of the mice to walk 30±7 cm further when analysed at same age of SOD1G93A mice and by their ability to move for a more extended period of time (FIG. 13e). These data suggest that promotion of muscle satellite cell activity and hypertrophy through mIGF-1 can be considered as an alternate therapeutic approach to counteract muscle wasting associated with ALS disease.

An activated calcineurin isoform is induced in SOD1G93A×MLC/mIGF-1 muscle. It has been reported that mutant SOD1 interferes directly with the protein phosphatase calcineurin-A (CnA) activity, supporting a role for calcineurin-regulated biochemical pathways in the pathogenesis of ALS (91). In addition, in skeletal muscle CnA has been implicated in myocyte hypertrophy (92) and fiber type conversion (93), inducing a signaling cascade that plays an important role in tissue remodeling after injury (94). In a study of selective CnA subunit isoform expression, it was recently established that the alternatively spliced variant CnA-β1 is up-regulated in regenerating skeletal muscle fibers (ms in preparation).

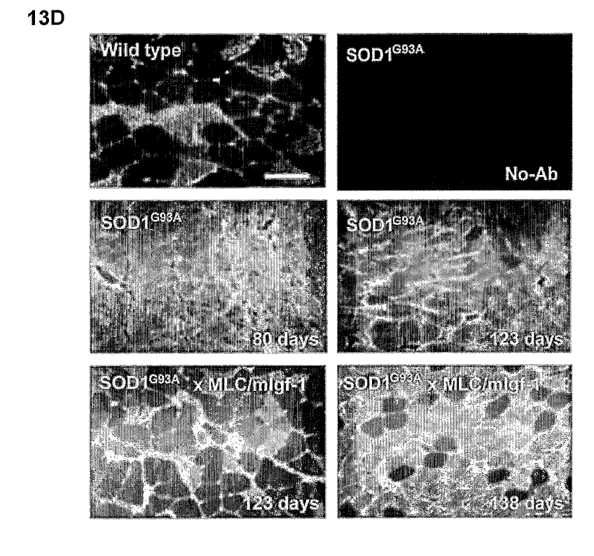

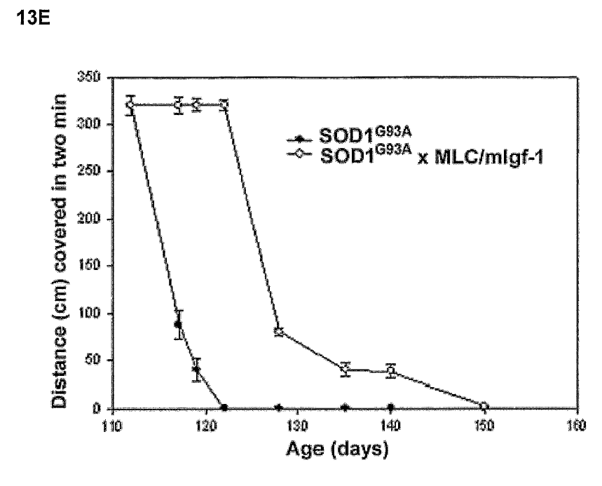

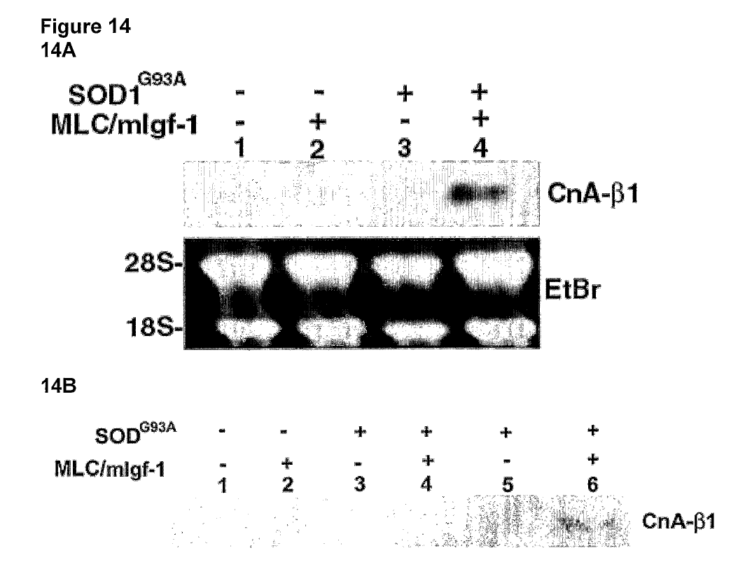

In this study it was verified whether the activation of satellite cells and the maintenance of muscle phenotype involved the induction of CnA-β1 expression. Although the normally low levels of CnA-β1 expression were not raised in SOD1G93A muscles (FIG. 14a lane 3; FIG. 14b lane 5, FIG. 14c), or in uninjured wild type and MLC/mIGF-1 muscle (FIGS. 14a and 14b, lane 1 and 2), SODG93AmIGF-1 regenerating muscle dramatically increased CnA-β1 transcripts (63%±5%) (FIG. 14a lane 4) and nuclear protein (52%±7%) (FIG. 3b lane 6, FIG. 3c) after onset of clinical symptoms and its expression remained at high levels even at end stage of disease. CnA-β1 represents a potential molecular player underlying the prolongation of muscle integrity even after denervation.

### Preservation of Neuromuscular Junctions in SODG93AmIGF-1 Mice

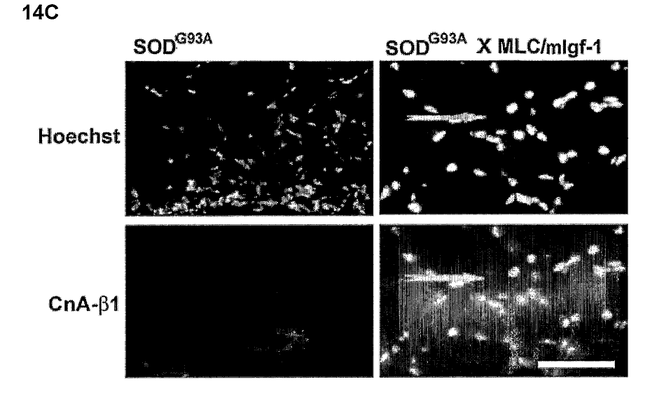

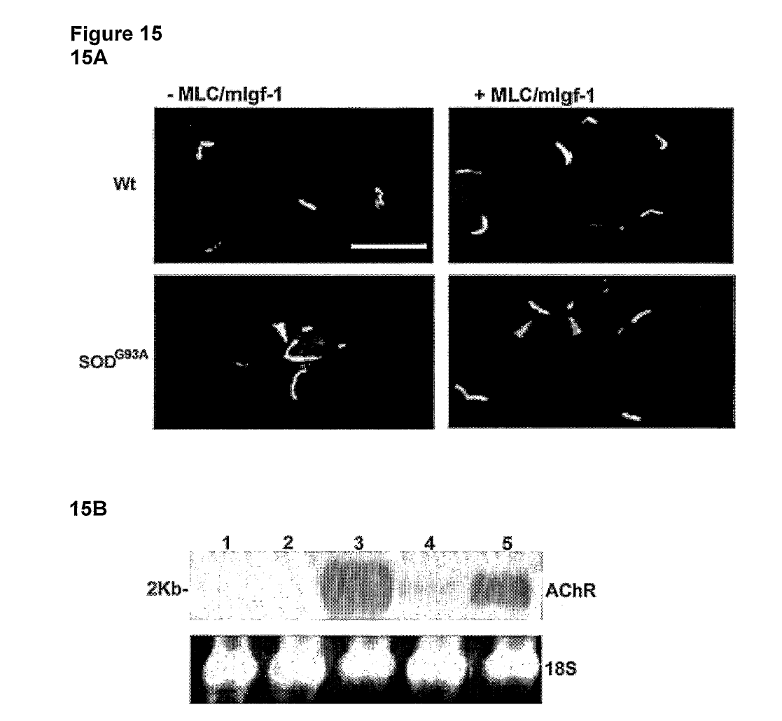

Alterations in motor neuronal activity typical of denervated muscle and motor neuron diseases also affects the configuration of neuromuscular junctions in SOD1G93A mice, characterised by the diffusion of acetylcholine receptor (AChR) postsynaptic clusters (FIG. 15a, yellow arrow). At 123 days, SOD1G93A paralyzed muscle showed 65%±0.19σ of diffuse AChR expression, whereas AChR cluster aggregates (FIG. 15a, red arrows) were preserved in muscles of age matched SODG93AmIGF-1 mice, which showed only 3.3%±0.4σ of diffuse AChR expression. At comparable end-stage disease, SODG93AmIGF-1 muscle displayed only 30%±0.20σ of diffuse AchR expression. These results were confirmed by Northern blot analysis (FIG. 15b); high AchR expression levels in SOD1G93A muscle were reduced in SODG93AmIGF-1 mice at all stages observed.

Densitometric analysis (n=6) revealed that AChR mRNA expression in SOD1G93A paralysed muscle (123 days) was 56%±5% higher than that observed in age matched SODG93AmIGF-1 mice; whereas the increase in mRNA expression in SOD1G93A mice was of 27%±6% when SOD1G93A and SODG93AmIGF-1 mice where analyzed at comparable end-stage disease. This suggests that mIGF-1 delays the progression of the disease, stabilizing the innervation of muscle fibers. The localization of AChR clusters at the endplate requires the expression of agrin, a large proteoglycan in the synaptic cleft that plays an important role in the maintenance of the molecular architecture of the postsynaptic membrane (95). Agrin expression showed a dramatic down-regulation in paralyzed SOD1G93A muscle compared to SODG93AmIGF-1 muscle (FIG. 15c) analyzed at comparable end-stage disease, further underscoring a role for local expression of IGF-1 in the maintenance of muscle innervation. Muscle-restricted mIGF-1 prolongs motor neuronal function in SOD1G93Amice.

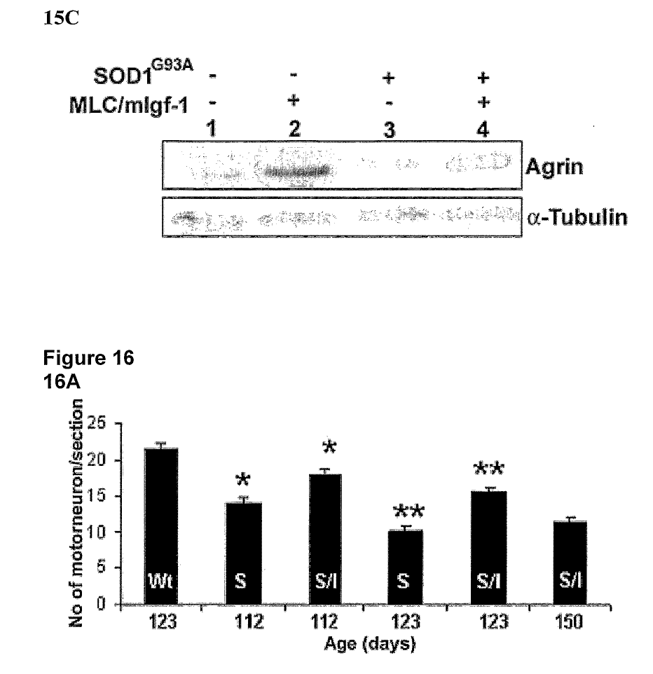

The extent to which muscle-restricted mIGF-1 expression preserves the motorneuron during the progression of ALS disease was then determined. Histological analysis of the ventral spinal cord revealed that SOD1G93A mice (n=7) presented a progressive reduction in the number of motor neuron from clinical onset to end stage disease. Specifically, SOD1G93A mice showed a reduction of 37% and 55% in the number of motorneurons at clinical onset (112 days) and at end stage disease (123 days) respectively (FIG. 16a). In contrast mIGF-1 expression induced motorneuron survival in SODG93AmIGF-1 mice (n=7) at all age and stage observed with significant differences at 112 and 123 days of age (FIG. 16a). One of the prominent markers of motor neuron dysfunction in ALS mice is the activation of astrocytes and microglia, leading to motor weakness and neural loss (96). Comparable patterns of GFAP immunoreactivity were found in spinal cords of SOD1G93A (n=6) and SODG93AmIGF-1 (n=6) transgenic mice before the symptom onset (28 days) (FIG. 16a panels A-B, panel D-insert lanes 1, 2). However, at paralysis stage (123 days) the spinal cord of SOD1G93A mice demonstrated a marked increase in astroglial activation (FIG. 16b panel C), with an increase in GFAP expression of about 35%±3% (FIG. 16b panel D-insert, lane 3) compared with the GFAP expression levels displayed in the spinal cord of age matched SODG93AmIGF-1 transgenic mice (FIG. 16b panel D and insert lane 4). At comparable end stage disease, there were no significant differences in GFAP expression between SOD1G93A and SODG93AmIGF-1 mice, although SOD1G93A mice continued to have a 13% more GFAP expression as compared to SOD1G93AmIGF-1 mice (data not shown).

The activation of astroglia can be correlated with the expression of certain cytokines, such as TNF-α, which enhances the response to inflammatory states and contributes to the progression of neurological dysfunction in SOD1 G93A mice (97).

While TNF-α expression was normally undetectable in the CNS of healthy mice (FIG. 16c, lanes 1, 2), it was accumulated in the spinal cord of SOD1G93A mice at paralysis stage (123 days) (FIG. 16c lane 3). In contrast, TNF-α expression was not apparent in the spinal cord of SODG93AmIGF-1 transgenic mice (FIG. 16c lane 4). This suggests that MLC/mIGF-1 hypertrophic muscle functions as a protective tissue for the CNS, modulating reactive astrocytosis and inflammatory cytokines that normally exacerbate the pathogenesis of ALS disease.

These results suggest that ALS is a “multi-systemic” disease in which the alteration in structural, physiological and metabolic parameters in different cell types (muscle, motorneurons, glia) may act synergistically to exacerbate the disease and evidences a functional cross-talk between neuronal and not neuronal cells (98).

Therefore, the present study serves to refocus therapeutic strategies to attenuate motor neuronal degradation towards skeletal muscle. It remains to be determined whether the dramatic prolongation of CNS tissue integrity in SODG93AmIGF-1 mice derives from the direct retrograde transport of transgenic mIgf-1, or indirect action either through distal activation endogenous IGF-1 expression, or through other trophic factors secreted by SODG93AmIGF-1 muscle.

In a previous retrograde transport of the AAV-IGF-1 vector from muscle to motor neuron was deemed necessary to achieve the therapeutic effects, since the same gene delivered to the muscle by a lentiviral vector, which cannot retrotransport, proved ineffective. The results of the present study suggest other explanations. An undefined IGF-1 cDNA used by Kaspar et al (99) may have encoded the more prevalent circulating gene product, which once secreted from the muscle, is not associated with extracellular matrix and disperses in the circulation (87). In contrast, the mIGF-1 isoform used in the present and previous studies (20;86;88;89) does not enter the circulation, but accumulates in the tissue of synthesis where its autocrine/paracrine function is concentrated. Thus, the importance of IGF-1 isoform choice in designing therapeutic strategies cannot be overstressed, since their diverse biological activities lead to radically different outcomes (87).

Whatever the mode of action, the feasibility of localized synthesis of the mIGF-1 isoform to activate survival mechanisms in distal damaged tissues represents a powerful approach to counteract the degeneration of both muscle and motor neuron in ALS disease.

### Material and Methods

### Mice

SODG93A transgenic mice (Jackson Laboratory) express the human mutant SOD1G93A allele containing the Gly93-->Ala (G93A) substitution, driven by its endogenous human promoter (100). The SOD1G93A B6J mice were crossed with MLC/mIGF-1 FVB mice (86) for 7 different generations to obtain SODG93AmIGF-1 B6J inbred transgenic mice. The animals were housed in a temperature-controlled (22° C.) room with a 12:12 h light-dark cycle.

### Walk Test

The walk test was performed in a scaled ramp, accordingly to the method reported by Gurney et al (100). The mice were allowed to explore the cage for 1 minute and then they were left to walk for 2 minutes. The hind feet of the mice were painted with ink and the track left by the mice were recorded on a paper tape. The test was performed in horizontal, laminar flow hood to maintain barrier conditions.

### Histological and Immunofluorescence Analysis

Muscle tissue was embedded in TBS-tissue freezing medium and frozen in nitrogen-cooled isopentane. For histological analysis, 7 μm tissue cryosections were fixed in 4% paraformaldehyde and stained with hematoxylin/eosin. For immunofluorescence analysis, 7 μm tissue sections were fixed with 4% paraformaldehyde, washed in PBS with 1% BSA and 0.2% Triton X, pre-incubated 1 hr in 10% goat serum at R.T. and incubated overnight with primary antibodies: neonatal Myosin Heavy Chain (neo-MyHC), MyHC-slo), MyHC-fas), Alexa Fluor™ 488 conjugated α Bungarotoxi), CnA-β), GFA). Nuclei were visualized by Hoechst staining. Stained cells were observed under an inverted microscope (model Axioskop 2 plus; Carl Zeiss Microimaging, Inc) using 20× or 40× lenses, and images were processed using Axiovision 3.1).

### RNA Preparation and Northern Analysis.

RNA was extracted from muscles by RNA-TRIZOL-kit (Gibco BRL). Total RNA was separated in 1.3% agarose gel and hybridized as previously described (Musarò and Rosenthal, 1999).

### RT-PCR Analysis

RNA from spinal cord of wildtype, MLC/mIgf-1, SODG93A and SODG93AmIGF-1 transgenic mice were used in RT-PCR assay. The following oligonucleotides were used: TNF-α sense 5′CCCAGACCCTCACACACTCAGAT3′ and anti sense 5′TTGTCCCTTGAAGAGAACCTG3′; β-Actin sense 5′GTGGGCCGCTCTAGGCACAA3′ and anti sense 5′CTCTTTGATGTCACGCACGATTTC3′.

### Protein Extraction and Western Blot Analysis

Protein extraction was performed in lysis buffer (50 mM Tris-HCl pH7.4, 1% w/v Triton ×100, 0.25% Sodium Deoxycholate, 150 mM Sodium Chloride, 1 mM Phenylmethylsulfonyl Fluoride, 1 μg/ml Aprotinin, 1 μg/ml Leupeptin, 1 μg/ml Pepstatin, 1 mM Sodium Orthovanadate, 1 mM Sodium Fluoride). Equal amounts of protein from each muscle lysate were separated in SDS polyacrylamide gel and transferred onto a Hybond C Extra nitrocellulose membrane. Filters were blotted with antibodies against human-SOD, myogenin, desmin; neo-MyHC, Agrin.

### Example 4

### Comparison of Signal Peptide and E Peptide Functions

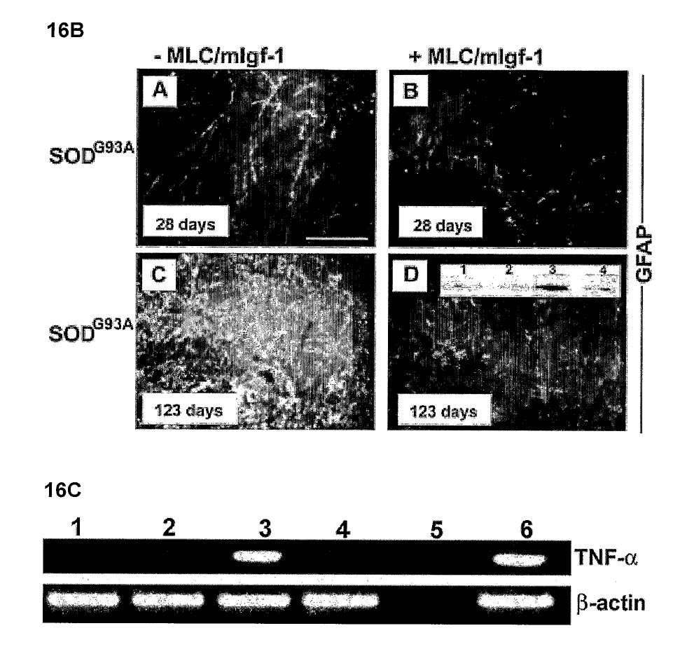

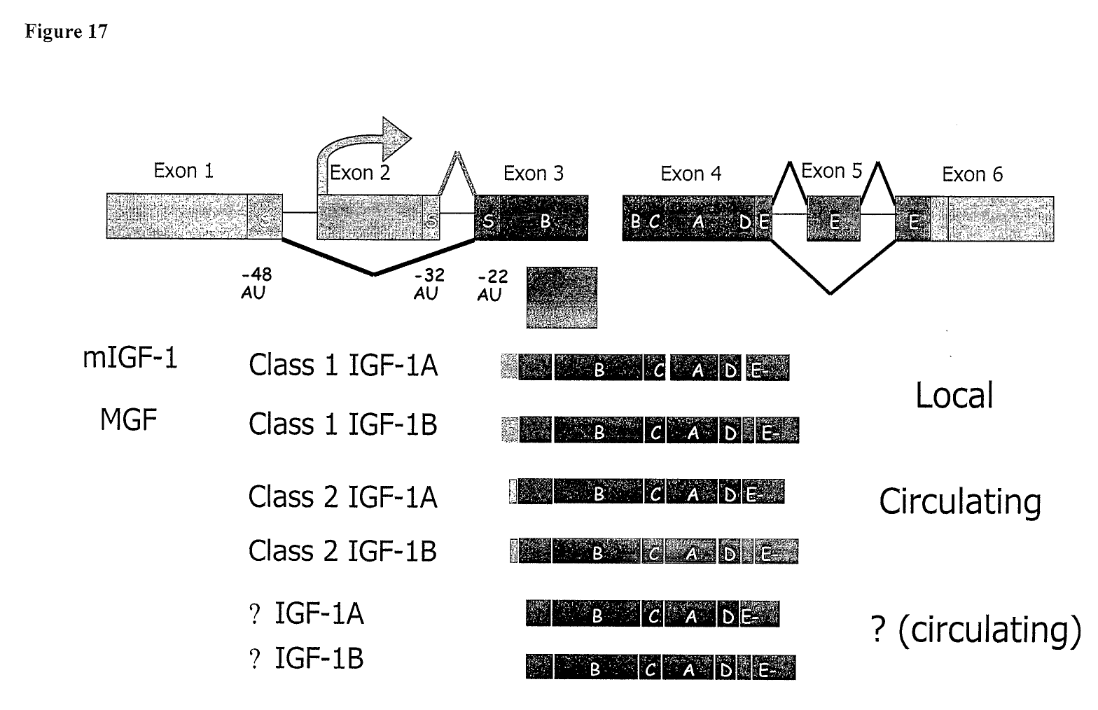

To assess the various effects of the different signal peptides and E peptides a series of studies were carried out involving direct comparisons of the IGF-1 isoforms in various biological systems. FIG. 17 shows a schematic representation of the IGF-1 gene and the various signal/E peptide isoforms.

Table 5 shows the usage of the various IGF-1 isoforms, in different muscle types, in wildtype mice.

TABLE 5
Normal mouse IGF-1 isoform usage.

| | | | Aged muscle | |
|---|---|---|---|---|
| | Wildtype | Exercised | (only in | Dystrophic |
| Construct | muscle | muscle | diaghragm) | muscle |
| Class 1 IGF-1A | + | ++ | + | ++ |
| Class 1 IGF-1B | − | − | + | + |
| Class 2 IGF-1A | − | − | + | − |
| Class 2 IGF-1B | − | − | + | ++ |

### IGF-1 Signal Peptides Control Myoblast Differentiation

To assess the effect of the IGF-1 signal peptides on myoblast differentiation kinetics, L6 proliferating mononucleated myoblast cultures were transfected with MLC/IGF-1A contained in a muscle specific expression vector. IGF-1A was expressed in conjunction with either the class 1, 2, or 3 signal peptide.

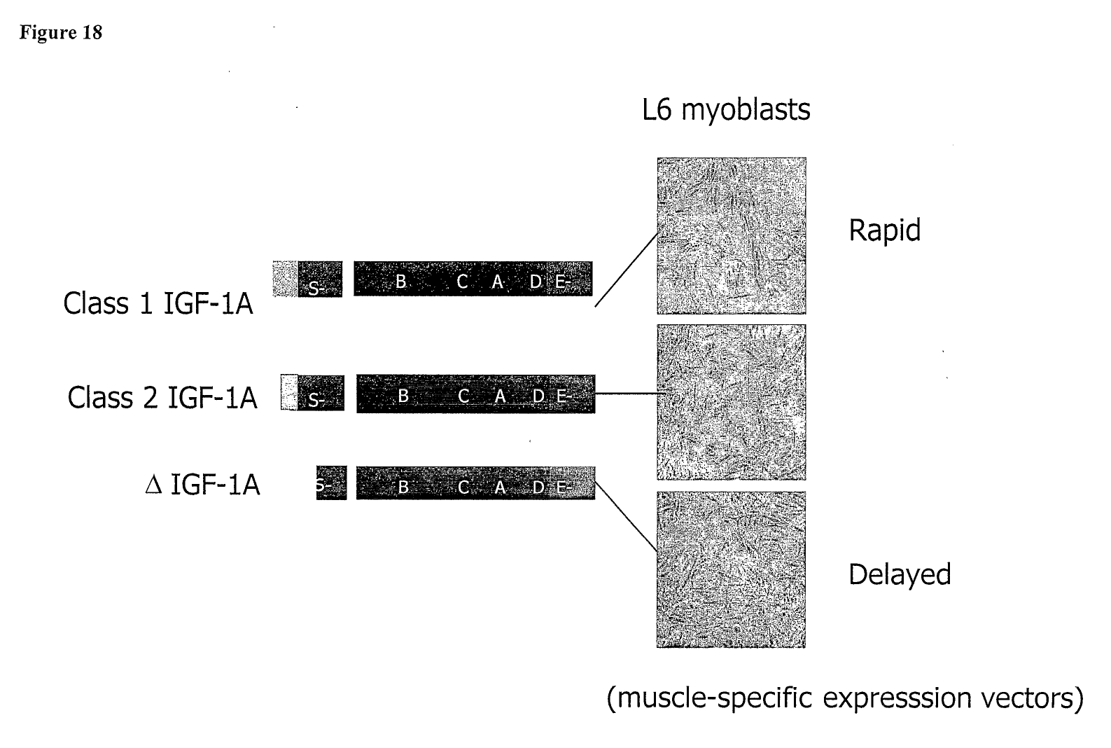

FIG. 18 shows the effect of each of the constructs on myoblast differentiation. Class 1 IGF-1-1 A causes L6 myoblasts to undergo rapid differentiation. Class 2 IGF-1A has little effect on L6 differentiation. Class 3 IGF-1A (ΔIGF-1A) causes delayed differentiation in L6 myoblasts.

These results suggest that the fate, and subsequent function of the IGF-1 peptide, is controlled by the various signal peptides.

Different Phenotypes Associated with E Peptides

To assess the effect of the IGF-1 E peptides on myoblast growth, L6 proliferating mononucleated myoblast cultures were transfected with MLC/Class 1 IGF-1A or MLC/Class 1 IGF-1B contained in a post-mitotic expression vector.

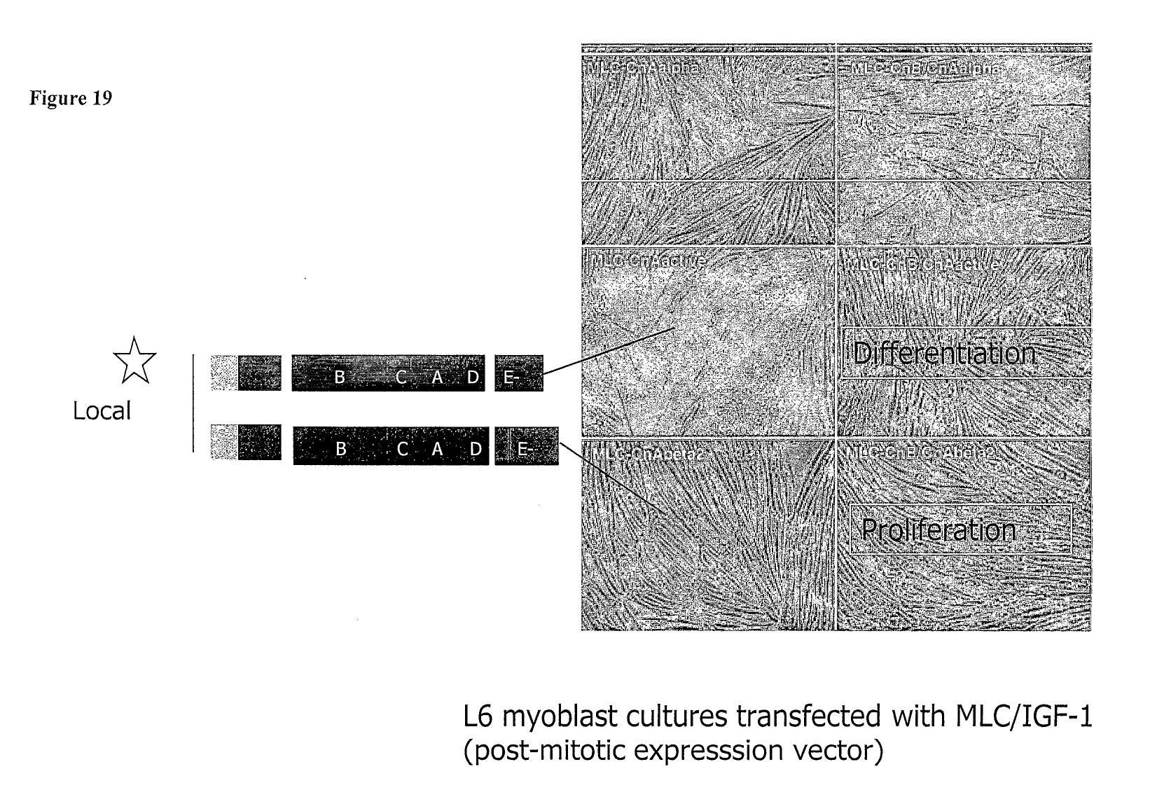

FIG. 19 shows the differing effect of the two constructs. Class 1 IGF-1A induces cellular differentiation in L6 myoblasts. In contrast, the Class 1 IGF-1B construct induces cellular proliferation.

These results suggest that the IGF-1 E peptides are responsible for controlling the different functions of the IGF-1 peptide.

### Testing IGF-1 Isoform Function In Vivo.

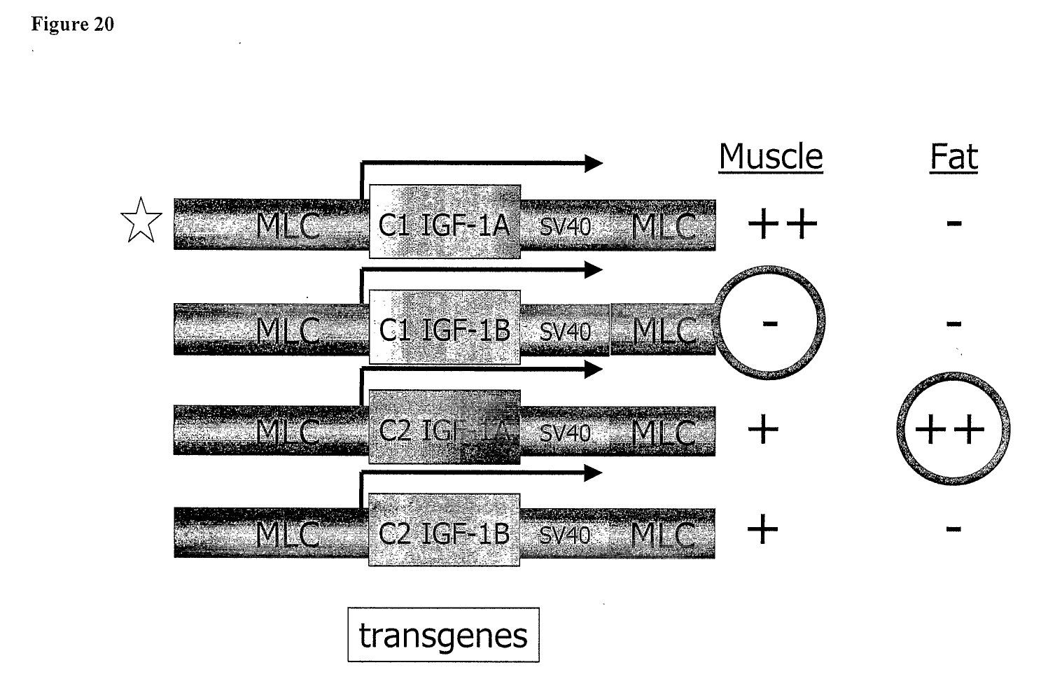

To assess the effects of the varying IGF-1 signal peptides in conjunction with the E peptides in vivo, transgenic mice were engineered following the methodology detailed in the previous examples. A total of six constructs were generated, representing the major IGF-1 isoforms (FIG. 20).

Transgenic mice expressing the Class 1 IGF-1A (mIGF-1) isoform show a hypertrophic response in skeletal muscle and a consequential increase in muscle mass. In comparison, transgenic mice expressing the Class 1 IGF-1B isoform show no increase in muscle hypertrophy. Tables 6-8 show phosphoproteins that are up-regulated, down-regulated or unaltered in Class 1 IGF-1A transgenic mouse muscle.

TABLE 6
Phosphoproteins up-regulated in Class
1 IGF-1A transgenic mouse muscle

| Description | % change |
|---|---|
| AMP-activated protein kinase alpha (T172) | 162 |
| Bruton's tyrosine kinase (Y223) | 24 |
| Cyclin-dependent kinase 1 (Y15) | 9 |
| Etk (BMX) (Y40) | 17 |
| Raf (S259) (60) | 16 |
| Phosphoinositide-dependent protein kinase 1 (S241) | 53 |
| mTOR (S2448) | 112 |
| p70 S6 kinase (T421/T424) | 254 |
| Retinoblastoma Protein (S780) | 36 |

TABLE 7
Phosphoproteins down-regulated in Class
1 IGF-1A transgenic mouse muscle

| Description | % change |
|---|---|
| Protein kinase C alpha/beta (T638) | −4 |
| Protein kinase C delta (T505) | −70 |
| Protein kinase theta (T538) | −8 |
| Protein kinase D (Protein kinase mu) (S916) | −37 |
| PKC-related kinase 1 (T778) | −29 |
| PKC-related kinase 2 (T816) | −50 |
| Raf (S259) (70) | −10 |
| Protein kinase B (T308) (Akt) | −4 |
| Glycogen synthase kinase-3 alpha (S21) | −50 |
| Glycogen synthase kinase-3 beta (S9) | −18 |
| I-kappa-B kinase beta (S181) | −18 |
| Lyn (Y507) (44) | −5 |
| Lyn (Y507) (46) | −13 |
| MAPK/Erk kinase 1/2 (S217/221) | −60 |
| MKK3/6(1) (S189/S207) | −50 |

TABLE 8
Phosphoproteins unaltered in Class
1 IGF-1A transgenic mouse muscle

| Description | % change |
|---|---|
| eIF4E binding protein (S65) (16) | 0 |
| eIF4E binding protein (S65) (17) | 0 |
| eIF4E binding protein (S65) (18) | 0 |
| CaMKII (T286) | 0 |
| Cyclin-dependent kinase 1 (T161) | 0 |
| I-kappaB kinase alpha (S180) | 0 |
| MAP kinase activated protein kinase 2 (T334) | 0 |
| MKK6 (2) (S207) | 0 |
| MAP kinase interacting kinase 1 (T197/202) | 0 |
| p38 MAPK (T180/Y182) | 0 |
| p70 S6 kinase (T389) | 0 |
| p85 S6 kinase 2 (T412) | 0 |
| p85 S6 kinase 2 (T444/S447) | 0 |
| 90 kDa Ribosomal S6 Kinases (S380) | 0 |

Transgenic mice expressing the Class 2 IGF-1A isoform exhibit a mildly hypertrophic phenotype in conjunction with a significant increase in adipose tissue. In comparison, transgenic mice expressing the Class 2 IGF-1B isoform exhibit a mildly hypertrophic phenotype with no increase in adipose tissue.

Several inferences can be drawn from these results:
1. Of the local (Class 1) isoforms, the Ea peptide-containing isoform has the most dramatic effect on local tissue. Since the two locally acting isoforms differ only by their E-peptide, a specific role for the Ea peptide can be predicted.
2. Of the circulating (Class 2) isoforms, the Ea peptide-containing isoform has the most dramatic anabolic effect on distal tissues, which implies that it travels to those tissues with the Ea peptide still attached to the IGF-1 peptide. From this it appears that adipose tissue may be the most sensitive to circulating IGF-1A.

From these inferences it can be concluded that the Ea and Eb peptides have different effects on regulation and differentiation of cells, illustrated here with effects on both muscle and adipose tissue.

### Example 5

### The Differential Role of IGF-1 Isoforms in Skeletal Muscle

In order to gain a greater understanding of the differential role of the IGF-1 isoforms and the specific functions of both the various signal peptides and E peptides, the studies described in example 4 were continued in more detail.

Two main approaches were taken to attempt to understand the function of different IGF-1 isoforms. Initially four IGF-1 isoforms were transiently over-expressed in a doxocycline-inducible manner in L6E9 cells, a myogenic cell line that doesn't express endogenous IGF-1. To further elucidate the in vivo effects of IGF-1 isoform, six transgenic mouse lines, each over-expressing one of the IGF-1 isoforms in skeletal muscle were generated and analyzed for their effect on the skeletal muscle phenotype. Another tool for understanding IGF-1 function has been the generation of IGF-1 inducible transgenic mice. Selected isoforms were cloned into a doxocycline-inducible vector and transgenic animals were generated and crossed with a skeletal muscle-specific inducer mouse to achieve timed IGF-1 transgene expression.

### Testing IGF-1 Isoform Function In Vitro

To test the function of IGF-1 isoforms in vitro, the “Tet-on” system was applied (101)(102), in which the gene encoding a modified tetracycline repressor protein (reverse tetracycline transactivator (rtTA)) is expressed via a minimal human CMV promoter, while the different IGF-1 isoforms are under the control of a rtTA responsive target promoter. The cell line of choice was the L6E9 cell line, a subclone of the parental rat neonatal myogenic line, which does not express endogenous IGF-1 but expresses the IGF-1 receptor. Therefore this cell line is a good system for analyzing the effect of single IGF-1 isoforms on myoblast proliferation and differentiation. L6E9 cells were double-transfected with the inducible IGF-1 encoding constructs and the rtTA-encoding inducer plasmid. Induction of IGF-1 isoform expression was achieved by administration of doxocycline. Cells were kept in growth medium for one day after transfection and then shifted to differentiation medium for four days. Presence of IGF-1 was confirmed by RT-PCR and Western blot throughout growth and the differentiation process.

When compared for their proliferative status during growth, Class 1 IGF-1A and Class 2 IGF-1A showed lower levels of phosphorylated Histone H3 in comparison to mock transfected cells, indicating that these two isoforms may have weaker effects on proliferation then the other four IGF-1 isoforms. Screening for involvement of different MAP kinases showed subtle differences in the activation by the various IGF-1 isoforms, which need to be further evaluated. Two downstream targets of the PI3 kinase pathway, Akt and S6 ribosomal protein, were analyzed and showed mild (Akt) to high (S6 ribosomal protein) increase of phosphorylation in comparison to mock transfected cells. The induction in both cases was comparable between the differently transfected lines. Analysis of the differentiation kinetics in all four transfectants points towards a prominent role of Class 1 IGF-1A (=mIGF-1) in accelerating the differentiation process. Cells transfected with this isoform were the first to stop proliferating upon the shift to differentiation medium (DM), started to fuse at day 2 in DM, and showed a stronger up-regulation of myogenic markers, like myogenin.

### Testing IGF-1 Isoform Function In Vivo

To gain further insight into the role of different IGF-1 isoforms in skeletal muscle in vivo, six transgenic mouse lines over-expressing one of the six IGF-1 isoforms were generated. Skeletal muscle-specificity is achieved by driving the transgene from the skeletal muscle-specific myosin light chain (MLC) 1/3 promoter and enhancer (FIG. 20).

### Analysis of Transgenic Founders

Offspring of all generated founders of each transgenic line (1-4 founders per line) were analyzed for transgene expression by Northern Blot. Where possible, 2 founders of each line have been selected due to high and comparable transgene expression. mRNA and protein analysis of individual muscle groups revealed high to moderate transgene expression levels, depending on the muscle fiber distribution of the examined muscle. Due to the expression pattern of the myosin light chain promoter, transgene expression is highest in the fast IIB fibers, although lower levels are also expressed in fast 2× and 2A fibers. Therefore fast muscles, like the quadriceps or the gastrocnemius showed higher transgene expression than mixed or slow muscles, such as the diaphragm or the soleus. No transgene expression was detected in any non-skeletal muscle tissue, such as heart, brain, liver, kidneys, or spleen. Analysis of endogenous IGF-1 isoform expression by Northern Blot and reverse transcription PCR in response to over-expression of a given IGF-1 isoform showed no alterations between WT and transgenic littermates.

### General Phenotype of Selected Transgenic Lines

For further comparison to the previously well characterised transgenic line MLC/mIGF-1 (=Class 1 IGF-1A) (83), the three lines representing the remaining predominant isoforms: MLC/Class 1 IGF-1B, MLC/Class 2 IGF-1A, and MLC/Class 2 IGF-1B were focused on. All selected transgenic lines were viable and appeared normal throughout development. As previously described (83), the MLC/mIGF-1 transgenic line showed skeletal muscle fiber hypertrophy, with an increase of muscle mass of over 50% and a decreased body fat content (83). Although these mice showed a pronounced increase in muscle mass, they did not change their total body weight.

MLC/Class 2 IGF-1B showed no difference in body weight up to an age of six months. Over-expression of MLC/Class 1 IGF-1B showed no effect on the total body weight at the age of one and three months, while by the age of six months, the body weight was significantly decreased. In contrast, MLC/Class 2 IGF-1A transgenic animals showed a significant increase in body weight already by the age of one and three months, which was maintained up to the age of six months. None of the transgenic lines showed any influence on the weight of distal organs like heart, spleen, kidney, brain, or liver throughout the monitored ages (one, three, and six months).

### Changes in Circulating IGF-1 Levels

IGF-1 has been shown to act either as a circulating hormone or as a local growth factor. It is widely accepted that the circulating versus local distribution of IGF-1 isoforms is dependent on the specific signal peptide. The mechanism behind this distinction was tested using the different MLC/IGF-1 isoform transgenic animals, where all four isoforms were over-expressed specifically in skeletal muscle. All MLC/IGF-1 isoform transgenic lines were analyzed for changes in circulating IGF-1 levels. Since all transgenes are of mouse origin and cannot specifically be detected, total amounts of IGF-1 were monitored. Plasma was collected from one- and six-months-old animals and screened by ELISA for IGF-1. By the age of one month, all lines showed elevated levels of total circulating IGF-1, which did not reach significance. Most surprisingly MLC/Class 1 IGF-1A and MLC/Class 1 IGF-1B showed a significant increase in total circulating IGF-1 by the age of six month, while MLC/Class 2 IGF-1A and MLC/Class 2 IGF-1B still showed elevated levels that did not reach significance, suggesting that at least when over-expressed in skeletal muscle, Class 1 rather then Class 2 isoforms influence total IGF-1 levels in the circulation. This effect becomes more pronounced with age, when endogenous IGF-1 levels actually decrease. These results support the importance of the signal peptide choice in determining the fate of the processed peptide.

### Skeletal Muscle Phenotype of Selected Transgenic Lines

The previously described transgenic line, over-expressing Class 1 IGF-1A (=mIGF-1), showed skeletal muscle fiber hypertrophy, first detectable at neonatal day 10, increasing into adulthood, when the transgenic mice have developed hypertrophic trunk and limb musculature with little or no body fat (83).

An elevated skeletal muscle weight was evident at 10 days of age for MLC/Class 2 IGF-1A and MLC/Class 2 IGF-1B, and increased significantly with age for MLC/Class 2 IGF-1A. MLC/Class 2 IGF-1B transgenic muscles showed a less pronounced, but still significant increase in skeletal muscle weight, which was maintained at comparable levels throughout the monitored ages. In contrast to both of the Class 2 transgenic lines, MLC/Class 1 IGF-1B didn't show significant changes in skeletal muscle weight until six months of age, when skeletal muscle groups showed a very moderate, but significant increase in muscle mass.

To determine whether the increase in skeletal muscle weight can be correlated to an increase of muscle fiber size, as reported for the MLC/mIGF-1 animals, six-months-old WT and transgenic mice of each line have been analyzed for differences in the single fiber cross sectional area (CSA) of the Tibialis Anterior (T.A.) and Extensor Digitorum Longus (E.D.L.) muscles. T.A. and E.D.L. both have a high content of fast type IIB fibers, which express MLC at the highest level. MLC/Class 2 IGF-1A and MLC/Class 2 IGF-1B showed a significant increase of fast fiber CSA in T.A., as well as E.D.L. muscles. In contrast, MLC/Class 1 IGF-1B didn't show a significant increase in the CSA of these muscles, despite the moderate but significant increase of muscle weight by the age of six months. However, when single fiber CSA was measured in six-month-old animals, a slight shift toward a higher percentage of bigger fibers was noticed in the MLC/Class 1 IGF-1B transgenic muscles, which might explain the weight increase of the different muscle groups. In these animals the CSA of the whole muscle was measured as well to determine if a higher percentage of bigger fibers was enough to increase the CSA of the whole muscle groups and therewith could account for an increased muscle mass. T.A. and E.D.L. showed an increase in CSA, which wasn't significant but still might explain the increased muscle mass of these animals.

Interestingly, MLC/Class 2 IGF-1A also showed a significant increase of the CSA of intermediate and slow fibers in T.A. and E.D.L. muscles, indicating that this isoform might be capable of functioning in a more paracrine way and thereby can act on adjacent intermediate and slow fibers to induce a hypertrophic response. Single fiber CSA measurements of the soleus muscle, which is mainly comprised of slow and intermediate fibers and shows very low levels of MLC expression, revealed no differences to WT samples in all transgenic lines. Fiber type composition was unchanged in all transgenic lines and all analyzed muscles.

### Skeletal Muscle Physiology of Selected IGF-1 Transgenic Lines

Comparison of titanic force of Class 2 IGF-1A and Class 2 IGF-1B EDL muscles revealed a significant increase in strength of Class 2 IGF-1A over wildtype (60%) whereas Class 2 IGF-1B did not have significant increased in strength despite mild muscle hypertrophy. Since the two Class 2 isoforms differ only by their E-peptide, these results provide further support for the inventors' proposal that the IGF-1 E peptides are responsible for controlling the different functions of the IGF-1 peptide.

### Influence on Other Components of the IGF-System

Even though two different IGF-1 receptors have been described, it is well established that IGF-1 function is exclusively mediated by the IGF-1 type 1 receptor (IGF-1R). Over-expression of IGF-1 could saturate the receptor and lead to down-regulation of transcriptional activity. To exclude the possibility that over-expression of different IGF-1 isoforms could interfere with IGF-1 receptor (IGF-1R) expression levels, Northern Blot analysis of IGF-1R mRNA levels was carried out on six-months-old WT and transgenic mice of each line. No differences could be detected, indicating that IGF-1R transcript levels are not influenced in the skeletal muscle of the transgenic animals.

Other components of the IGF system are the seven different IGF-1 binding proteins (IGFBPs), which are able to either inhibit or potentiate IGF-1 action and thereby add another level to IGF-1 regulation. Affimetrix analysis of all transgenic lines at one month of age revealed an up-regulation of IGFBP-5 in MLC/Class 1 IGF-1B and was therefore the first candidate among the IGFBPs to be analyzed. IGFBP-5 can enhance IGF-1 action when bound to extracellular matrix, while it is cleaved to a biologically inactive fragment when it is soluble. Posttranslational glycosylation of IGFBP-5 has also been shown to modify the affinity to IGF-1 (103). In one-month-, as well as six-month-old animals, protein levels of non-modified IGFBP-5 were comparable to WT levels. However, MLC/Class 1 IGF-1A and MLC/Class 1 IGF-1B showed a slight induction of a higher molecular weight band, which represents a glycosylated form of IGFBP-5.

### Signal Transduction Pathways

Different pathways have been implicated in IGF-1 mediated hypertrophy. While Rommel et al. (104) and Bodine at el. (60) have shown the PI3 Kinase pathway and its downstream effectors Akt and GSK3 to be responsible for myocyte hypertrophy, Musaróet al. (83) have suggested the calcineurin pathway and the downstream effectors NF-ATc1 and Gata-2 as the mediator of skeletal muscle hypertrophy. To investigate whether similar activation of NF-AT and Gata-2 can be seen in the different transgenic lines, Northern and Western analysis has been performed. In contrast to the MLC/mIGF-1 muscles, Gata-2 expression was unchanged in the quadriceps of both, MLC/Class 2 IGF-LA and MLC/Class 2 IGF-1B animals, indicating that in the case of over-expressing Class 2 IGF-1 isoforms, other pathways must be implicated in the induction of hypertrophy. In MLC/Class 1 IGF-1B animals, which did not show hypertrophic muscle fibers, Gata-2 expression was expectedly not effected as well.

To further elucidate which pathway might be involved in mediating the effects of the different IGF-1 isoforms, quadriceps samples of one-month-old animals from all transgenic lines were screened for phosphorylation-mediated activation of a broad range of key kinases involved in downstream signaling of IGF-1.

Downstream effectors of the PI3 kinase pathway, like Akt, PDK1, and GSK3α and β were up-regulated in both Class 2 IGF-1 isoforms, while not affected or down-regulated in both Class 1 IGF-1 isoforms. Down-regulation of Akt in Class 1 isoforms confirm recent findings of Song et al., (105) showing that Akt is not involved in mediating mIGF-1 (Class 1 IGF-1A) induced hypertrophy. On the other hand, the same group reported an increased phosphorylation of PDK1, mTOR, and p70S6K, which was not seen for either Class 1 IGF-LA, or Class 1 IGF-1B. These results imply a signal peptide-specific difference in the induction of signal transduction pathways.

### Regeneration of Transgenic IGF-1 Isoform Muscles

MLC/mIGF-1 transgenic animals have been reported to show enhanced regeneration upon cardiotoxin-induced skeletal muscle injury (83). For analysis of regenerative capacity, MLC/Class 1 IGF-1B and MLC/Class 2 IGF-1A transgenic animals were focused on, since these two transgenic lines showed the most prominent phenotype in skeletal muscle. Cardiotoxin was injected into the T.A. muscle and animals were analyzed at two, five, and ten days after the injections. MLC/Class 1 IGF-1B mice show a significantly enhanced regenerative response compared to wildtype mice. After two days, massive injury was seen in both, WT and transgenic muscles. Five days after injection, the WT muscle showed high levels of infiltrating mononuclear cells, indicating inflammatory processes. The proliferative response of muscle satellite cells was also initiated at this time point, characterized by small myofibers with centralized nuclei. In contrast to the WT, the MLC/Class 1 IGF-1B muscle showed a dramatic increase in the formation of new fibers, as well as a less severe inflammatory response. After 10 days the transgenic muscle had undergone almost complete regeneration. New fibers had reached normal size, no fibrotic tissue formation was detectable, and mononuclear cells were cleared, indicating that the inflammatory processes have been resolved. In the WT muscle the majority of newly formed fibers were still quite small, mononuclear cells were still visible and some fibrotic tissue formation was seen. These results imply that Class 1 IGF-1B enhances the regenerative process in the same way as mIGF-1, where an increased proliferation of satellite cells (83), an increased recruitment of bone marrow cells (24), and a down-regulation of the inflammatory response is seen.

This is an important result as it demonstrates that an IGF-1 isoform, which does not induce skeletal muscle hypertrophy, is nevertheless capable of enhancing the regenerative response in response to traumatic injury. It shows that enhanced regeneration is not connected to the hypertrophic phenotype seen in MLC/mIGF-1 animals.

### CONCLUSIONS

The initial in vitro experiments on the effect of IGF-1 isoforms on myoblast proliferation suggest that Ea-containing isoforms (Class 1 IGF-1A and Class 2 IGF-1A) are less efficient in stimulation of proliferation, as shown by a down-regulation of phospho Histone H3, a marker of mitosis. In contrast, Eb-containing isoforms (Class 1 IGF-1B and Class 2 IGF-1B) showed similar levels of phospho H3 when compared to the mock transfected cells.

Further in vivo evidence for a distinct role of the E-peptides in mediating IGF-1 function, arises from the analysis of transgenic animals, over-expressing the different IGF-1 isoforms in a post-mitotic manner. Pronounced skeletal muscle hypertrophy, as seen in the MLC/Class 1 IGF-1 (MLC/mIGF-1) animals previously described (83), was also seen in MLC/Class 2 IGF-1A animals. By contrast, Eb-containing isoforms show either mild (MLC/Class 2 IGF-1B) or no significant hypertrophy (MLC/Class 1 IGF-1B), implying that the nature of the E-peptide plays an important role in determination of IGF-1 isoform function. As with the original MLC/mIGF-1 animals, Class 2 IGF-1A animals are significantly stronger compared to wildtype, and compared to Class 2 IGF-1B animals. Apart from the presence of different E-peptides, IGF-1 isoforms also differ by the signal peptide (=Class), providing another means of influencing IGF-1 isoform function (106). As described previously (107-110), Class 2 isoforms are considered to be the endocrine version of IGF-1, while Class 1 isoforms have been thought to have a local role. The animal models presented in this work, where IGF-1 expression is restricted to skeletal muscle, allowed us to address which of the IGF-1 isoforms stays in the tissue of origin. Measuring total IGF-1 levels in the circulation revealed an up-regulation of circulating IGF-1 in response to over-expressing Class 1, rather than Class 2 isoforms. These results contradict the current technical literature and may suggest a peculiarity to skeletal muscle or imply the involvement of other tissue-specific factors, such as IGFBPs, in the determination of the fate of IGF-1. The results certainly confirm that the signal peptide plays an important role in determining IGF-1 function.

Further support for the importance of the signal peptide comes from differences seen in the activation of signal transduction pathways, where Class 2 IGF-1 isoforms show up-regulation of certain pathways that are not affected by Class 1 isoforms.

These studies further suggest that some IGF-1 functions are mediated by the presence of a certain E-peptide, whilst others are correlated to the presence of a certain signal peptide.

### Example 6

### The Effect of mIGF-1 on Inflammatory Response During Muscle Regeneration and in Muscular Dystrophy

Inflammation is a critical component of muscle regeneration and is an important phase necessary to activate the stem cell compartment and therefore regeneration. Nevertheless, the inflammatory response must be resolved to proceed towards muscle repair. In fact, muscle regeneration fails when muscle injury is associated with altered spatial distribution of inflammatory cells, altered identity of the inflammatory infiltrate and altered temporal pattern.

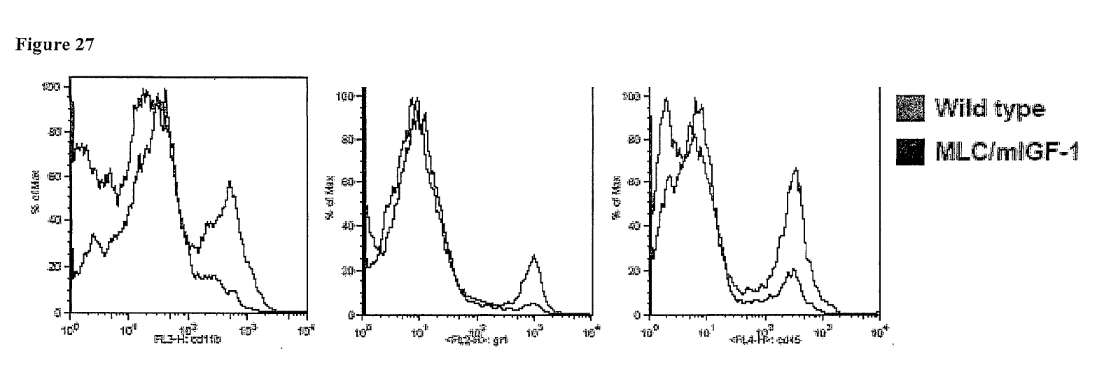

We accumulated evidence demonstrating that mIGF-1 accelerates the timing of regeneration and reduced the amount of mononucleated infiltrating cells at five days post-injury, while the regenerative capacity of injured wild type muscle was substantially delayed (FIG. 27).

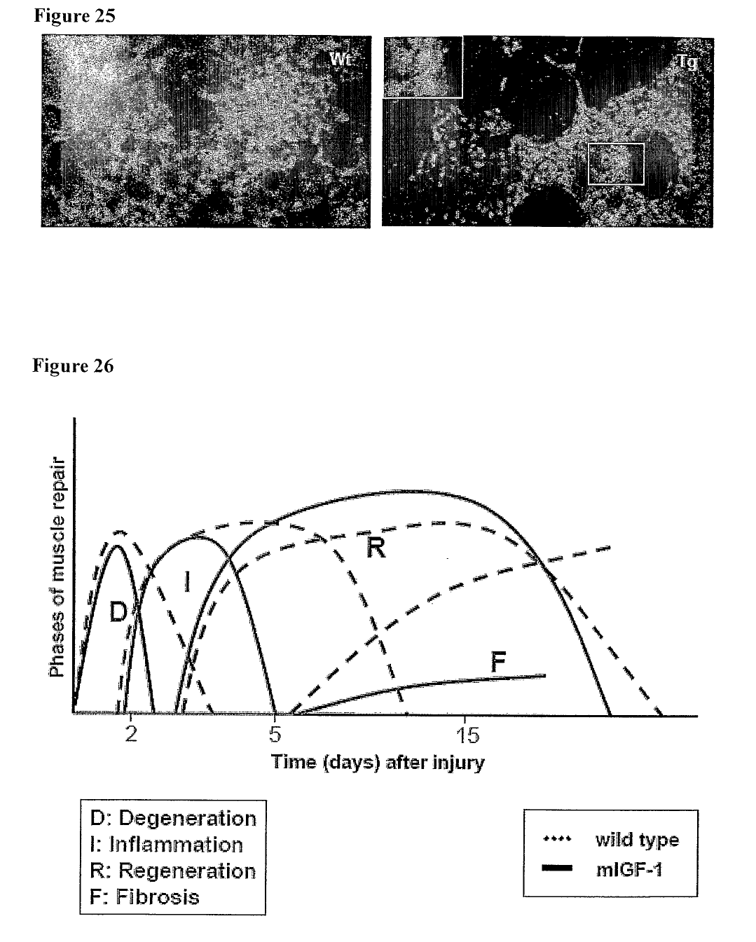

Our results demonstrated that the local expression of mIGF-1 improves the regenerative phase increasing the pool of satellite cells (FIG. 25) and modulating the inflammatory response of injured skeletal muscle (FIG. 26).

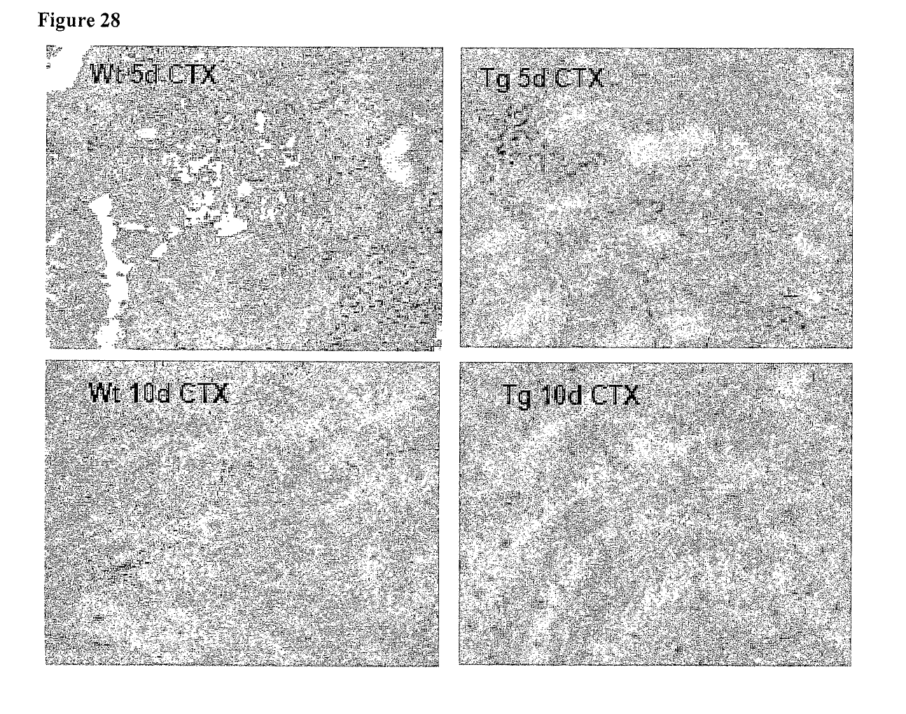

In particular, quantitative RT-PCR and proteomic analysis demonstrated that mIGF-1 modulates inflammatory cytokines, such as MCP1, MCP2, MIP-1α, and MIP-1β at early stages, stimulating a qualitative environment for a complete functional recovery. Indeed, the muscle architecture of MLC/mIGF-1 injured mice was rapidly and almost completely restored compared to wild type muscle (FIG. 28).

These results suggest that mIGF-1 modulates inflammatory cytokines at early stages, stimulating a qualitative environment for a complete functional recovery. This was confirmed by analyzing the effect of mIGF-1 in the mdx, dystrophic mouse model. It has been reported that that hematopoietic stem cell (HCS) migration into sites of injury may be a mechanism by which damaged tissues are repaired. However, this is a rare event and presents limitations for efficient tissue repair. It has been reported that the poor recruitment of HSC into the dystrophic muscle of the mdx mouse is the major obstacle for muscle regeneration and therefore for the rescue of the genetic disease.

It is proposed that the recruitment and mobilization of stem cells is not the only limitation for stem cell-mediated muscle regeneration in dystrophic muscle. Among potential parameters that have impeded the generation of satisfactory protocols for stem cell therapy is the activation of deleterious signal transduction pathways by dystrophic milieu. In this context, our working hypothesis was that dystrophic microenvironment renders unproductive the stem cell-mediated therapy.

FACS analysis revealed that dystrophic muscle was able to recruit a large population of cells expressing markers of the hematopoietic stem cell, such as Sca1, c-kit and CD45. In this context, the recruitment of stem cells is not the critical parameter for the success of stem cell therapy. Preliminary evidence suggests that the microenvironment plays a pivotal role limiting the stem cell mediated therapy.

RT-PCR analysis revealed that inflammation was modulated by mIGF-1 expression in mdx/mIGF-1 transgenic mice. This opened the question whether the mIGF-1 expression, that improves the dystrophic environment, also stimulates the regenerative capacity of stem cells.

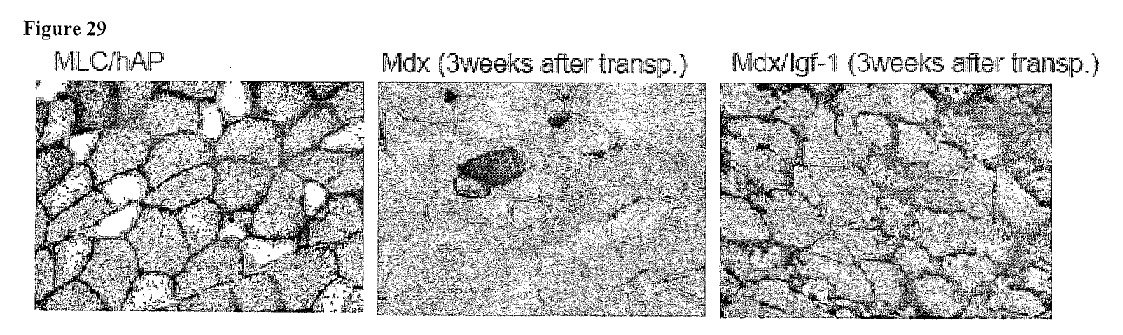

Stem cells isolated from the bone marrow of MLC/hAP mouse were transplanted into the mdx and mdx/mIgf-1 dystrophic muscle to investigate whether the mIGF-1 expression, that improves the dystrophic environment, also stimulates the regenerative capacity of stem cells. The MLC/hAP mouse is a good model to follow the differentiative fate of bone marrow stem cells, since these stem cells will activate the transgene hAP only when transdifferentiated into skeletal muscle. Histological analysis revealed that transplanted stem cells massively participated in muscle regeneration only in mdx/mIGF-1 dystrophic mice (FIG. 29).

These data confirm the hypothesis that mIGF-1 promotes a qualitative environment for an efficient muscle regeneration.

### REFERENCES

{1} Blundell, T. L. and Humbel, R. E., 1980, Nature 287:781-787
{2} Ebberink, R. H. M. et al., 1989, Biol. Bull 177:176-182
{3} Smit, A. B., et al., 1989, Nature 331:535-538
{4} Froesh, E. R., in Insulin-like Growth Factors/Somatomediams, de Gruyter, S. M. (ed.), New York, pp. 18-29 (1983)
{5} Lowe, M. W. Jr., in Insulin-like Growth Factors:Molecular and Cellular Aspects, LeRoith, D. (ed.), Boca Raton, Fla., CRC Press, pp 49-80 (1991)
{6} Adamo M. L., Ben-Hur H., Roberts C. T., Jr., LeRoith D., (1991) Regulation of start site usage in the leader exons of the rat insulin-like growth factor-I gene by development, fasting, and diabetes, Mol Endocrinol 5:1677-1686
{7} Simmons J G, Van Wyk J J, Hoyt E C, Lund P K. (1993) Multiple transcription start sites in the rat insulin-like growth factor-I gene give rise to IGF-I mRNAs that encode different IGF-I precursors and are processed differently in vitro. Growth Factors. 9(3):205-21
{8} Yang H., Adamo M. L., Koval A. P. et al., (1995) Alternative leader sequences in insulin-like growth factor I mRNAs modulate translational efficiency and encode multiple signal peptides, Mol Endocrinol 9:1380-1395
{9} Bell G. I., Stempien M. M., Fong N. M., (1986) Rall L. B., Sequences of liver cDNAs encoding two different mouse insulin-like growth factor I precursors, Nucleic Acid Research 14:7873-7882
{10} Shimatsu A., Rotwein P., (1987) Mosaic evolution of the insulin-like growth factors. Organization, sequence, and expression of the rat insulin-like growth factor I gene, J Biol Chem 262:7894-7900
{11} Roberts C. T. Jr., Lasky S. R., Lowe W. L., Jr., Seaman W. T., LeRoith D., (1987) Molecular cloning of rat insulin-like growth factor I complementary deoxyribonucleic acids: Differential messenger ribonucleic acid processing and regulation by growth hormone in extrahepatic tissues, Mol Endocrinol 1:243-248.
{12} Le Bouc Y., Dreyer D., Jaeger F., Binoux M., Sondermeyer P., (1986) Complete characterization of the human IGF-I nucleotide sequence isolated from a newly constructed adult liver cDNA library, FEBS Lett 196:108-112.
{13} Lund P. K., Hoyt E. C., Van Wyk J. J., (1989) The size heterogeneity of rat insulin-like growth factor-I mRNAs is due primarily to differences in the length of 3′-untranslated sequence, Mol Endocrinol 3:2054-2061.
{14} Ito H, Hiroe M, Hirata Y, Tsujino M, Adachi S, Shichiri M, Koike A, Nogami A, Marumo F. (1993) Insulin-like growth factor-I induces hypertrophy with enhanced expression of muscle specific genes in cultured rat cardiomyocytes, Circulation 87:1715-21.
{15} Kajstura J, Cheng W, Reiss K, Anversa P. (1994) The IGF-1-IGF-1 receptor system modulates myocyte proliferation but not myocyte cellular hypertrophy in vitro. Exp Cell Res. 215:273-83.
{16} Bark T H, McNurlan M A, Lang C H, Garlick P J. (1998) Increased protein synthesis after acute IGF-I or insulin infusion is localized to muscle in mice. Am J Physiol. 275:E118-23.
{17} Cittadini A, Stromer H, Katz S E, Clark R, Moses A C, Morgan J P, Douglas P S. (1996) Differential cardiac effects of growth hormone and insulin-like growth factor-1 in the rat. A combined in vivo and in vitro evaluation. Circulation. 93:800-9.
{18} Reiss K, Cheng W, Ferber A, Kajstura J, Li P, Li B, Olivetti G, Homcy C J, Baserga R, Anversa P. (1996) Overexpression of insulin-like growth factor-1 in the heart is coupled with myocyte proliferation in transgenic mice. Proc Natl Acad Sci USA. 93:8630-5.
{19} Delaughter M C, Taffet G E, Fiorotto M L, Entman M L, Schwartz R J. 1999 Local insulin-like growth factor I expression induces physiologic, then pathologic, cardiac hypertrophy in transgenic mice. FASEB J. 13:1923-9.
{20} Barton-Davis E R, Shoturma D I, Musaro A, Rosenthal N, Sweeney H L. 1998 Viral mediated expression of insulin-like growth factor I blocks the aging-related loss of skeletal muscle function. Proc Natl Acad Sci U S A. 95:15603-7.
{21} Musaro A, Giacinti C, Borsellino G, Dobrowolny G, Pelosi L, Cairns L, Ottolenghi S, Cossu G, Bernardi G, Battistini L, Molinaro M, Rosenthal N. (2004) Stem cell-mediated muscle regeneration is enhanced by local isoform of insulin-like growth factor 1. Proc Natl Acad Sci USA. 101:1206-10.
{22} Paul A C, Rosenthal N. (2002) Different modes of hypertrophy in skeletal muscle fibers. J Cell Biol. 156(4):751-60.
{23} Barton E R, Morris L, Musaro A, Rosenthal N, Sweeney H L. (2002) Muscle-specific expression of insulin-like growth factor I counters muscle decline in mdx mice. J Cell Biol. 157:137-48.
{24} Musaro A, Giacinti C, Borsellino G, Dobrowolny G, Pelosi L, Cairns L, Ottolenghi S, Cossu G, Bernardi G, Battistini L, Molinaro M, Rosenthal N. (2004) Stem cell-mediated muscle regeneration is enhanced by local isoform of insulin-like growth factor 1. Proc Natl Acad Sci USA. 101:1206-10.
{25} Ibba (1996) Biotechnol Genet Eng Rev 13:197-216
{26} Sambrook (1989) Molecular Cloning; A Laboratory Manual ISBN: 0879695773
{27} Fernandez et al. (1998) eds Gene expression systems. Using nature for the art of expression ISBN: 0122538404
{28} Summers et al. (1987) Texas Agricultural Experiment Station Bulletin No. 1555
{29} U.S. Pat. No. 5,693,506
{30} U.S. Pat. No. 5,659,122
{31} U.S. Pat. No. 5,608,143
{32} Zenk (1991) Phytochemistry 30:3861-3863
{33} Bodanszky (1993) Principles of Peptide Synthesis ISBN: 0387564314
{34} Fields et al (1997) Methods in Enzymology 289: Solid-Phase Peptide Synthesis ISBN: 0121821900
{35} Chan & White (2000) Fmoc Solid Phase Peptide Synthesis ISBN: 0199637245
{36} Kullmann (1987) Enzymatic Peptide Synthesis ISBN: 0849368413
{37} Gennaro (2000) Remington: The Science and Practice of Pharmacy 20th ed, ISBN: 0683306472
{38} WO 02/36161
{39} Almeida & Alpar (1996) J Drug Targeting 3:455-467
{40} Merly F, Lescaudron L, Rouaud T, Crossin F, Gardahaut M F (1999) Macrophages enhance muscle satellite cell proliferation and delay their differentiation. Muscle Nerve. 22:724-32.
{41} Camargo F D, Green R, Capetanaki Y, Jackson K A, Goodell M A, Capetenaki Y. (2003) Single hematopoietic stem cells generate skeletal muscle through myeloid intermediates. Nat Med. 9(12): 1520-7.
{42} Berkner, K. L., Curr. Top. Microbiol. Immunol., 158, 39-66 (1992)
{43} Muzyczka, N., Curr. Top. Microbiol. Immunol., 158, 97-129 (1992)
{44} U.S. Pat. No. 5,252,479
{45} Chapter 20, Gene Therapy and other Molecular Genetic-based Therapeutic Approaches, (and references cited therein) in Human Molecular Genetics (1996), T Strachan and A P Read, BIOS Scientific Publishers Ltd
{46} Current Protocols in Molecular Biology (F M Ausubel et al, eds. 1987) Supplement 30
{47} Smith & Waterman (1981) Adv Appl Math 2: 482-489
{48} Hambrecht R, Schulze P C, Gielen S, Linke A, Mobius-Winkler S, et al. (2002) Reduction of insulin-like growth factor-I expression in the skeletal muscle of noncachectic patients with chronic heart failure. J Am Coll Cardiol 39(7): 1175-1181.
{49} Schulze P C, Gielen S, Adams V, Linke A, Mobius-Winkler S, et al. (2003) Muscular levels of proinflammatory cytokines correlate with a reduced expression of insulin-like growth factor-I in chronic heart failure. Basic Res Cardiol 98(4): 267-274.
{50} Musaro A, McCullagh K, Paul A, Houghton L, Dobrowolny G, et al. (2001) Localized IGF-1 transgene expression sustains hypertrophy and regeneration in senescent skeletal muscle. Nat Genet 27(2): 195-200.
{51} Paul A C, Rosenthal N (2002) Different modes of hypertrophy in skeletal muscle fibers. J Cell Biol 156(4): 751-760.
{52} Sandri M, Sandri C, Gilbert A, Skurk C, Calabria E, et al. (2004) Foxo transcription factors induce the atrophy-related ubiquitin ligase atrogin-1 and cause skeletal muscle atrophy. Cell 117(3): 399-412.
{53} Florini J, Ewton D, Coolican S (1996) Growth hormone and the insulin-like growth factor system in myogenesis. Endocrine Rev 17: 481-517.
{54} Fiorotto M L, Schwartz R J, Delaughter M C (2003) Persistent IGF-I overexpression in skeletal muscle transiently enhances DNA accretion and growth. Faseb J 17(1): 59-60.
{55} Barton E R, Morris L, Musaro A, Rosenthal N, Sweeney H L (2002) Muscle-specific expression of insulin-like growth factor I counters muscle decline in mdx mice. J Cell Biol 157(1): 137-148.
{56} Subramaniam, A. et al. Tissue-specific regulation of the alpha-myosin heavy chain gene promoter in transgenic mice. J Biol Chem 266, 24613-20 (1991).
{57} Musaro, A. & Rosenthal, N. Maturation of the myogenic program is induced by postmitotic expression of insulin-like growth factor I. Mol Cell Biol 19, 3115-24 (1999).
{58} Orlic, D. et al. Mobilized bone marrow cells repair the infarcted heart, improving function and survival. Proc Natl Acad Sci USA 98, 10344-9 (2001).
{59} M. a. M. a. a. a. s. o. m. o. S. Online.
{60} S. C. Bodine et al., Nat Cell Biol 3, 1014-9 (November, 2001).
{61} D. J. Glass, Nat Cell Biol 5, 87-90 (February, 2003).
{62} J. Montagne et al, Science 285, 2126-9 (Sep. 24, 1999).
{63} R. Bohni et al., Cell 97, 865-75 (Jun. 25, 1999).
{64} H. Shima et al., Embo J 17, 6649-59 (Nov. 16, 1998).
{65}D. J. Glass, Trends Mol Med 9, 344-50 (August, 2003).
{66} N. Pullen et al., Science 279, 707-10 (Jan. 30, 1998).
{67} Hertweck, M., Gobel, C. & Baumeister, R. C. elegans SGK-1 is the critical component in the Akt/PKB kinase complex to control stress response and life span. Dev Cell 6, 577-88 (2004).
{68} Paradis, S. & Ruvkun, G. Caenorhabditis elegans Akt/PKB transduces insulin receptor-like signals from AGE-1 PI3 kinase to the DAF-16 transcription factor. Genes Dev 12, 2488-98 (1998).
{69} Frodin, M., Jensen, C. J., Merienne, K. & Gammeltoft, S. A phosphoserine-regulated docking site in the protein kinase RSK2 that recruits and activates PDK1. Embo J 19, 2924-34 (2000).
{70} G. Ren, O. Dewald, N. G. Frangogiannis, Curr Drug Targets Inflamm Allergy 2, 242-56 (September, 2003).
{71} K. W. Liechty, H. B. Kim, N. S. Adzick, T. M. Crombleholme, J Pediatr Surg 35, 866-72; discussion 872-3 (June, 2000).
{72} G. P. Dotto, Biochim Biophys Acta 1471, M43-56 (Jul. 31, 2000).
{73} S. A. Murray, H. Zheng, L. Gu, Z. X. Jim Xiao, Oncogene 22, 1703-11 (Mar. 20, 2003).
{74} Y. Nonomura, H. Kohsaka, K. Nagasaka, N. Miyasaka, J Immunol 171, 4913-9 (Nov. 1, 2003).
{75} H. Perlman et al., J Immunol 170, 838-45 (Jan. 15, 2003).
{76} J. P. Brockes, A. Kumar, C. P. Velloso, J Anat 199, 3-11 (July-August, 2001).
{77} K. D. Poss, L. G. Wilson, M. T. Keating, Science 298, 2188-90 (Dec. 13, 2002).
{78} J. O. Oberpriller, J. C. Oberpriller, D. G. Matz, M. H. Soonpaa, Ann N Y Acad Sci 752, 30-46 (Mar. 27, 1995).
{79} M. T. Longaker, Z. M. Peled, J. Chang, T. M. Krummel, Surgery 130, 785-7 (November, 2001).
{80} B. Nadal-Ginard, J. Kajstura, P. Anversa, A. Leri, J Clin Invest 111, 1457-9 (May, 2003).
{81} B. Nadal-Ginard, J. Kajstura, A. Leri, P. Anversa, Circ Res 92, 139-50 (Feb. 7, 2003).
{82} A. Musaro et al., Proc Natl Acad Sci USA 101, 1206-10 (Feb. 3, 2004).
{83} A. Musaro et al., Nat Genet. 27, 195-200 (February, 2001).
{84} Rossig, L., Badorff, C., Holzmann, Y., Zeiher, A. M. & Dimmeler, S. Glycogen synthase kinase-3 couples AKT-dependent signaling to the regulation of p21Cip1 degradation. J Biol Chem 277, 9684-9 (2002).
{85} Urbanek, K. et al. Cardiac stem cells possess growth factor-receptor systems that after activation regenerate the infarcted myocardium, improving ventricular function and long-term survival. Circ Res 97, 663-73 (2005).
{86} Musarò A, K. McCullagh, A. Paul, L. Houghton, G. Dobrowolny, M. Molinaro, E. R. Barton, H. L. Sweeney, and N. Rosenthal. 2001. Localized IGF-1 transgene expression sustains hypertrophy and regeneration in senescent skeletal muscle. Nat Genet. 27: 195-200.
{87} Musarò, A. and N. Rosenthal. 2002. The role of local Insulin-like Growth Factor-1 isoforms in the pathophysiology of skeletal muscle. Current Genomics 3: 149-162.
{88} Barton, E. R., L. Morris, A. Musaro, N. Rosenthal, and H. L. Sweeney. 2002. Muscle-specific expression of insulin-like growth factor I counters muscle decline in mdx mice. J Cell Biol. 157: 137-48.
{89} Musarò, A., C. Giacinti, G. Borsellino, G. Dobrowolny, L. Pelosi, L. Cairns, S. Ottolenghi, G. Bernardi, G. Cossu, L. Battistini, M. Molinaro, and N. Rosenthal. 2004. Muscle restricted expression of mIGF-1 enhances the recruitment of stem cells during muscle regeneration. Proc Natl Acad Sci USA. 101:1206-1210.
{90} Barton-Davis, E. R., D. I. Shoturma, A. Musaro, N. Rosenthal, and H. L. Sweeney. 1998. Viral mediated expression of insulin-like growth factor I blocks the aging-related loss of skeletal muscle function. Proc Natl Acad Sci USA. 95: 15603-7.
{91} Ferri A., R. Gabbianelli, A. Casciati, E. Paolucci, G. Rotilio, M. T. Carri. 2000. Calcineurin activity is regulated both by redox compounds and by mutant familial amyotrophic lateral sclerosissuperoxide dismutase. J Neurochem. 75: 606-13.
{92} Musaro, A., K. J. McCullagh, F. J. Naya, E. N. Olson, and N. Rosenthal. 1999. IGF-1 induces skeletal myocyte hypertrophy through calcineurin in association with GATA-2 and NF-ATc1. Nature. 400: 581-585.
{93} Chin E. R., E. N. Olson, J. A. Richardson, Q. Yang, C. Humphries, J. M. Shelton, H. Wu, W. Zhu, R. Bassel-Duby, and R. S. Williams. 1998. A calcineurin-dependent transcriptional pathway controls skeletal muscle fiber type. Genes Dev. 12: 2499-509.
{94} Sakuma K., J. Nishikawa, R. Nakao, K. Watanabe, T. Totsuka, H. Nakano, M. Sano, and M. (2003)
{95} McConville, J. and A. Vincent. 2002. Diseases of the neuromuscular junction. Curr Opin Pharmacol. 2: 296-301.
{96} Hall, E. D., J. A. Oostveen, and M. E. Gurney. 1998. Relationship of microglial and astrocytic activation to disease onset and progression in a transgenic model of familial ALS. Glia 23: 249-256.
{97} Elliott J L. 2001 Cytokine upregulation in a murine model of familial amyotrophic lateral sclerosis. Brain Res Mol Brain Res. 95: 172-178.
{98} Ferri A., M. Nencini, A. Casciati, M. Cozzolino, D. F. Angelini, P. Longone, A. Spalloni, G. Rotilio, and M. T Carri. 2004. Cell death in amyotrophic lateral sclerosis: interplay between neuronal and glial cells. FASEB J.
{99} Kaspar, B. K., J. Llado, N. Sherkat, J. D. Rothstein, and F. H. Gage. 2003. Retrograde viral delivery of IGF-1 prolongs survival in a mouse ALS model. Science. 301: 839-842.
{100} Gurney M. E., H. Pu, A. Y. Chiu, M. C. Dal Canto, C. Y. Polchow, D. D. Alexander, J. Caliendo, A. Hentati, Y. W. Kwon, H. X. and Deng. 1994. Motor neuron degeneration in mice that express a human Cu, Zn superoxide dismutase mutation. Science 264: 1772-1775.
{101} Gossen, M. and H. Bujard (1992). “Tight control of gene expression in mammalian cells by tetracycline-responsive promoters.” Proc Natl Acad Sci USA 89(12): 5547-51.
{102} Baron U, Bujard H. (2000) Tet repressor-based system for regulated gene expression in eukaryotic cells: principles and advances. Methods Enzymol. 327:401-21.
{103} Jones, J. I., Clemmons, D. R. (1995), Insulin-like growth factors and their binding proteins: biological actions. Endocr Rev 16(1): 3-34
{104} Rommel, C. et al., Mediation of IGF-1-induced skeletal myotube hypertrophy by PI(3)K/Akt/GSK3 pathways. Nat Cell Biol, 2001, 3(11):p. 1009-13
{105} Song, Y. H. et al. (2005). “Insulin-like growth factor-1-mediated skeletal muscle hypertrophy is characterized by increased mTOR/p70S6K signaling without increased Akt phosphorylation.” J Investig Med. 53(3):135-142
{106} Shavlakadze, T., Winn, N. Rosenthal, N., Grounds, M. (2005). “Reconciling data from transgenic mice that overexpress IGF-1 specifically in skeletal muscle.” Growth Hormone & IGF Res 15:4-18
{107} Lowe, W. L., Jr., C. T. Roberts, Jr., S. R. Lasky and D. LeRoith (1987). “Differential expression of alternative 5′ untranslated regions in mRNAs encoding rat insulin-like growth factor I.” Proc Natl Acad Sci U S A 84(24): 8946-50.
{108} Hoyt, E., Van Wyk, J J., Lund, P K. (1988). “Tissue and development specific regulation of a complex family of rat insulin-like growth factor I messenger ribonucleic acids.” Mol Endocrinol 2: 1077-1086.
{109} Lowe, W. L., Jr., S. R. Lasky, D. LeRoith and C. T. Roberts, Jr. (1988). “Distribution and regulation of rat insulin-like growth factor I messenger ribonucleic acids encoding alternative carboxyterminal E-peptides: evidence for differential processing and regulation in liver.” Mol Endocrinol 2(6): 528-35.
{110} Adamo, M. L., H. Ben-Hur, C. T. Roberts, Jr. and D. LeRoith (1991). “Regulation of start site usage in the leader exons of the rat insulin-like growth factor-I gene by development, fasting, and diabetes.” Mol Endocrinol 5(11): 1677-86.

1. A peptide having the formula NH₂-A-B—C-D-COOH, wherein: -A- is an optional N-terminus amino acid sequence consisting of a amino acids; —B— is an optional amino acid sequence consisting of b amino acids; —C— is a sequence derived from an IGF-1 Ea or Eb peptide; and -D- is an optional C-terminus amino acid sequence consisting of d amino acids.

2. A peptide according to claim 1, wherein —C— is a sequence derived from an IGF-1 Ea peptide.

3. A peptide according to claim 1, wherein —C— is a sequence derived from an IGF-1 Eb peptide.

4. A peptide according to any of claims 1-3, wherein the value of a, b and d is 0.

5. A peptide according to any of claims 1-3 or 5, wherein —B— is a sequence derived from the mature processed IGF-1 peptide.

6. A peptide according to claim 5, wherein the value of a and/or d is 0.

7. A peptide according to any of claims 1-3 or 5, wherein -A- is a Class 1 IGF-1 signal peptide.

8. A peptide according to any of claims 1-3 or 5, wherein -A- is a Class 2 IGF-1 signal peptide.

9. A peptide according to any of claims 1-3 or 5, wherein -A- is a Class 3 IGF-1 signal peptide.

10. A peptide according to any of claims 7-9, wherein the value of d is 0.

11. A peptide according to any of the preceding claims, wherein —C— comprises the amino acid sequence as recited in SEQ ID NO:2.

12. A peptide according to any of the preceding claims, wherein —C— comprises the amino acid sequence as recited in SEQ ID NO:4.

13. A peptide according to any of claims 1-3, 5, or 7-12, wherein -A- comprises the amino acid sequence as recited in SEQ ID NO:6.

14. A peptide according to any of claims 1-3, 5, or 7-12, wherein -A- comprises the amino acid sequence as recited in SEQ ID NO:8.

15. A peptide according to any of claims 1-3, 5, or 7-12, wherein -A- comprises the amino acid sequence as recited in SEQ ID NO:10.

16. A peptide according to any of claims 1-3, 5, or 7-12, wherein —B— comprises the amino acid sequence as recited in SEQ ID NO:12.

17. A purified nucleic acid molecule which encodes a polypeptide according to any one of the preceding claims.

18. A purified nucleic acid molecule according to claim 17, which comprises the nucleic acid sequence as recited in SEQ ID NO:1, SEQ ID NO:3, SEQ ID NO:5, SEQ ID NO:7, SEQ ID NO:9 or SEQ ID NO:11, or is a redundant equivalent or fragment thereof.

19. A purified nucleic acid molecule according to claim 18, which consists of the nucleic acid sequence as recited in SEQ ID NO:1, SEQ ID NO:3, SEQ ID NO:5, SEQ ID NO:7, SEQ ID NO:9 or SEQ ID NO:1, or is a redundant equivalent or fragment thereof.

20. A vector comprising a nucleic acid molecule as recited in any one of claims 17 to 19.

21. A host cell transformed with a vector according to claim 20.

22. Use of a peptide according to any one of claims 1-16, a nucleic acid molecule according to any one of claims 17-19, a vector according to claim 20 or a host cell according to claim 21, for the regulation of cellular growth or differentiation.

23. Use according to claim 22 wherein said regulation is of muscle tissue; nervous tissue; adipose tissue; cartilage; bone; hepatic tissue; kidney tissue; or skin.

24. Use according to claim 23 wherein said muscle tissue is skeletal, smooth or cardiac muscle.

25. Use of a peptide according to any of claims 1-16, a nucleic acid molecule according to any one of claims 17-19, a vector according to claim 19 or a host cell according to claim 21 in therapy.

26. Use of a peptide according to any of claims 1-16, a nucleic acid molecule according to any one of claims 17-19, a vector according to claim 20 or a host cell according to claim 21, in the manufacture of a medicament for the prevention or limitation of cellular trauma.

27. Use according to claim 26, wherein said cellular trauma is to muscle tissue; nervous tissue; adipose tissue; cartilage; bone; hepatic tissue; kidney tissue; or skin.

28. Use according to claim 26 or 27, wherein the damage is caused by crush injury; surgical damage; muscle tear injury; nerve damage; surgical damage; ischemia burns; bone fractures or UV damage.

29. Use according to claim 27 or 28, wherein said trauma to muscle tissue is to the myocardium.

30. Use according to any of claims 26-29 wherein said medicament is administered prior to said cellular trauma occurring in an attempt to prevent and reduce the damage.

31. Use according to any of claims 26-29 wherein said medicament is administered after said cellular trauma has occurred.

32. Use according to any of claims 26-31 wherein said peptide has the ability to induce a hypertrophic phenotype in said cells.

33. Use of a peptide according to any of claims 1-16, a nucleic acid molecule according to any one of claims 17-19, a vector according to claim 20 or a host cell according to claim 21, in the manufacture of a medicament for reducing muscular atrophy.

34. Use according to claim 33 wherein said muscular atrophy is caused by a degenerative disorder (cachexia); disuse of the muscle; sarcopenia; congestive heart failure or a stroke.

35. Use according to claim 34 wherein said disuse is caused by a restriction to normal muscle movement for a prolonged period of time or muscular paralysis.

36. Use according to claim 34 wherein the degenerative disorder is a neuromuscular disorder; a neurodegenerative disorder or a muscular dystrophy.

37. Use according to claim 34 wherein congestive heart failure includes cardiomyopathies; atherosclerosis; acute insult including myocarditis or myocardial infarction.

38. Use according to any of claims 34-37 wherein said medicament is administered as a preventative measure to slow said muscular atrophy or a therapeutic measure to both slow and reverse said muscular atrophy.

39. Use of a peptide according to any of claims 1-16, a nucleic acid molecule according to any one of claims 17-19, a vector according to claim 20 or a host cell according to claim 21, in the manufacture of a medicament for increasing muscular hypertrophy.

40. Use according to claim 39 wherein said medicament is administered to livestock to increase edible volume of the livestock.

41. Use according to claim 39 wherein said medicament is administered to a patient as an aid to physical therapy.

42. Use of a peptide of any one of claims 1-16, a nucleic acid molecule according to any one of claims 17-19, a vector according to claim 20 or a host cell according to claim 21, in the manufacture of a medicament for increasing adipose tissue.

43. Use according to claim 39 or 42 wherein said medicament is provided to help a patient gain weight after a severe illness, injury or continuing infection.

44. Use according to claim 42 wherein said medicament is used to treat Anorexia nervosa or Bulimia nervosa.

45. Use according to any of claims 22-44 wherein said medicament is administered orally, intravenously, intramuscularly, intra-arterially, intramedullary, intrathecally, intraventricularly, transdermally, subcutaneously, intraperitoneally, intranasally, enterally, topically, sublingually, intravaginally or rectally.

46. Use of claim 45 wherein said medicament is co-administered with thrombin.

47. Use according to claim 45 or 46 wherein said medicament is intended to act either at the site of administration or systemically.

48. A transgenic non-human animal that has been transformed to express higher levels of a polypeptide according to any one of claims 1 to 16.

49. A kit comprising one or more of: a peptide of any one of claims 1-16, a nucleic acid molecule according to any one of claims 17-19, a vector according to claim 20 or a host cell according to claim 21.

50. A kit according to claim 49, additionally comprising thrombin.
